SEQUENCE LISTING (8 sequences)

SEQ ID NO: 1 - DNA, 53 bp, Artificial Sequence
  Synthetically generated oligonucleotide
gtttgctcgacttcttaaattcaagagatttaagaagtcgagcaaactttttt

SEQ ID NO: 2 - DNA, 61 bp, Artificial Sequence
  Synthetically generated oligonucleotide
aattaaaaaagtttgctcgacttcttaaatctcttgaatttaagaagtcgagcaaacggcc

SEQ ID NO: 3 - DNA, 53 bp, Artificial Sequence
  Synthetically generated oligonucleotide
gtgcatcccatcagttctgttcaagagacagaactgatgggatgcactttttt

SEQ ID NO: 4 - DNA, 61 bp, Artificial Sequence
  Synthetically generated oligonucleotide
aattaaaaaagtgcatcccatcagttctgtctcttgaacagaactgatgggatgcacggcc

SEQ ID NO: 5 - DNA, 53 bp, Artificial Sequence
  Synthetically generated oligonucleotide
cccgggtttcaatatatttctcaagagaaaatatattgaaacccgggtttttt

SEQ ID NO: 6 - DNA, 61 bp, Artificial Sequence
  Synthetically generated oligonucleotide
aattaaaaaacccgggtttcaatatattttctcttgagaaatatattgaaacccgggggcc

SEQ ID NO: 7 - DNA, 53 bp, Artificial Sequence
  Synthetically generated oligonucleotide
tttaaacccggccctttggttcaagagaccaaagggccgggtttaaatttttt

SEQ ID NO: 8 - DNA, 61 bp, Artificial Sequence
  Synthetically generated oligonucleotide
aattaaaaaatttaaacccggccctttggtctcttgaaccaaagggccgggtttaaaggcc

(19) United States
(12) Patent Application Publication
(10) Pub. No.: US 2008/0171710 A1
(43) Pub. Date: Jul. 17, 2008

(54) The Redox/Fyn/c-Cbl Pathway

(75) Inventors: Mark Noble, Brighton, NY (US); Chris Proschel, Pittsford, NY (US); Zaibo Li, West Henrietta, NY (US); Brett Stevens, Rochester, NY (US); Yin Miranda Yang, Rochester, NY (US); Tiefei Dong, Rochester, NY (US)

Correspondence Address: FISH & RICHARDSON P.C., P.O BOX 1022, Minneapolis, MN, 55440-1022, US

(73) Assignee: UNIVERSITY OF ROCHESTER, Rochester, NY (US)

(21) Appl. No.: 11/623,919

(22) Filed: Jan. 17, 2007

(51) Int. Cl. A61K 31/7105; A61P 35/00; C12N 5/02; C12N 5/08; C12Q 1/02

(52) U.S. Cl. 514/44; 435/29; 435/375; 435/377

(57) ABSTRACT

Methods and compositions related to a novel cellular pathway, the redox/Fyn/c-Cbl pathway, include a variety of aspects. Provided herein are agents that selectively interrupt the pathway and methods of using the same. Also provided are screening methods used to identify a pro-oxidation toxicant or other agents and stimuli that affect the redox/Fyn/c-Cbl pathway. Methods of testing oxidation levels by evaluating aspects of the pathway are also provided.

### FEDERALLY SPONSORED RESEARCH OR DEVELOPMENT

The U.S. Government may have certain rights in this invention pursuant to Grant Nos. HD39702 and ES012708 awarded by the National Institutes of Health.

### BACKGROUND

Little is understood about common mechanistic features related to the disruption of normal cellular function and tire disruption of normal development caused by chemical toxicants and other environmental stimuli that can alter the normal behavior of cells. For example, in the field of toxicology, determining whether chemically diverse substances induce similar adverse effects at the cellular and molecular level is one of the central challenges. If the structural diversity of different toxicants, and of potential toxicants, means that each works through distinctive mechanisms, then this creates a potentially unsolvable challenge in developing means of screening the many tens of thousands of different, chemicals for which little or no toxicological information exists. In contrast, the identification of general principles or points of convergence that transcend the specific chemistries of individual substances has the potential of providing broadly relevant insights into the means by which toxicants disrupt normal development. If such principles were found to apply to the analysis of toxicant levels frequently encountered in the environment, this would be of even greater potential importance in providing efficient means of analyzing this diverse array of chemicals.

In addition, a more general concern regarding the search for general principles of toxicant action is whether such convergence, if it exists, would occur only at exposure levels that induce cell death or whether common mechanisms might be relevant to the understanding of more subtle effects of toxicant exposure, particularly during, critical developmental periods or in early stages of disease. Because development is a cumulative process, the effects of small changes in, e.g., progenitor cell division and/or differentiation, that are maintained over multiple cellular generations could have substantial effects on the organism. Such changes are poorly understood, however, at both cellular and molecular levels.

There is also a broad interest in many fields related to how changes in the cellular state alter cellular functions. The suppression of cell division is associated not just with exposure to toxic substances but also is central to the understanding of the processes that normally regulate precursor cell function. Understanding how the balance between self-renewing division of a precursor cell and the induction of its differentiation is one of the central challenges of precursor cell biology, with important ramifications for the development of the field of regenerative medicine.

### SUMMARY

Provided herein are methods and compositions related to a novel pathway, the redox/Fyn/c-Cbl pathway. Provided herein are agents that selectively interrupt the pathway. Also provided are methods of reducing the effects of oxidation in a cell comprising contacting a cell having an activated redox/fyn/c-Cbl pathway or a cell at risk for activation of the redox/fyn/c-Cbl pathway with an agent that selectively interrupts the redox/fyn/c-Cbl pathway. Methods of screening for a pro-oxidation toxicant, for concentrations of the toxicant that have a deleterious effect, and for agents that interrupt the redox/Fyn/c-Cbl pathway are provided. Furthermore, methods of promoting proliferation of a precursor cell and methods of treating cancer in a subject using an agent that selectively interrupts the redox/fyn/c-Cbl pathway are taught. Methods of testing oxidation levels by evaluating aspects of the pathway are also provided.

### DESCRIPTION OF DRAWINGS

FIG. 1 shows a diagrammatic summary of the role of redox regulation in modulating division and differentiation of 0-2A/OPCs. Progenitor cells are induced to divide by exposure to PDGF induced to divide by PDGF alone, progenitors undergo a limited number of divisions while asymmetrically generating oligodendrocytes. The balance between division and differentiation is modulated, however, by the intracellular redox state [9]. Cells that are more oxidized tend to differentiate, whereas those that are more reduced undergo more self-renewal. Pharmacological agents that make cells more oxidized induce differentiation of dividing O-2A/OPCs into oligodendrocytes. Similarly, signaling molecules that induce differentiation (e.g. TH) make cells more oxidized as a necessary part of their mechanism of action. In contrast, pharmacological agents that make cells more reduced promote self-renewal, and signaling molecules that enhance self-renewal (e.g., neurolrophin-3) make cells more reduced as a necessary part of their mechanism of action.

FIG. 2 shows the effect of MeHg exposure on O-2A/OPC oxidation, BrdU incorporation, and cell division in clonal assays. FIG. 2(A) shows the redox state of purified O-2A/OPCs grown in the presence of 10-ng/ml PDGF overnight Effects of MeHg on intracellular redox, state were determined by analysis of 2′,7′-dichlorodihydrofluorescein diacetate fluorescence emission in O-2A progenitors exposed to 20 nM MeHg for various lengths of time, as indicated. FIG. 2(B) shows BrdU incorporation when cells were plated in medium containing PDGF and then exposed to 20 nM MeHg for an additional 72 h. During the last 4 h of exposure, cultures were also exposed to BrdU. Cultures were then stained with A2B5 and anti-BrdU antibodies (to recognize all progenitors and those synthesizing DNA during the BrdU pulse, respectively). Results are presented as comparison with control cultures. FIGS. 2(C-E) show suppression of cell division by exposure to MeHg at the clonal level. Cells were treated as for (B), except for being plated at clonal density (as in [9,199]), Cultures were maintained for 6 d, and then 100 randomly chosen clones were analyzed for their composition (as in [9,14,199]). Data are presented, for all clones analyzed, in three-dimensions such that the x-axis equals the number of progenitors per clone, the z-axis equals the number of oligodendrocytes (Oligo) per clone, and the y-axis equals the number of clones with any given composition, in cultures exposed to MeHg, there was a decrease in the representation of large clones and a proportionate increase in the number of small clones and in clones containing oligodendrocytes. The effects of MeHg were prevented by co-exposure of cells to 1 mM N-acetyl-L-cysteine (NAC), which has pro-oxidant activity and is able to increase levels of intracellular glutathione. AH experiments were repeated at least three times, and all numerical values represent means±SD for triplicate data points.

FIG. 3 shows MeHg suppresses PDGF-mediated signaling from the nucleus back to the receptor. FIG. 3(A) shows data for progenitors transfected with an SRE-luciferase reporter construct and exposed to 30 nM MeHg (24 h). The MeHg treated progenitors showed significantly lower levels of reporter activity. A single asterisk (*) indicates p<0.05. FIG. 3(B) shows the data for cells grown as in (A) and analyzed for phosphorylation of Erk1/2. Cells exposed to MeHg showed reduced Erk1/2 phosphorylation. FIG. 3(C) shows data for cells transfected with an Nf-κB-luciferase reporter construct and treated as in (A), which had reduced Nf-κB transcriptional activity. Double asterisks (**) indicate p<0.01. FIG. 3(D) showed that cells grown as in (A) showed lower levels of Akt (Thr 308) phosphorylation. FIG. 3(E) showed that cells grown as in (A) also showed decreased phosphorylation of PDGFRα (as detected with anti-PDGFRα(pY742) antibody). All effects of MeHg were prevented by growth of cells in the additional presence of 1 mM NAC. All experiments were repeated at least three times, and all numerical values represent means±SD for triplicate data points. The “+” symbol indicates exposure of the cells to the indicated substance.

FIG. 4 shows MeHg effects are pathway specific and are associated with reduced levels of PDGFRα FIG. 4(A) shows MeHg does not inhibit Erk1/2 phosphorylation induced by exposure to NT-3. Cells were grown as in FIG. 2A, but exposed to NT-3 instead of PDGF. As shown, MeHg exposure did not reduce the extent of Erk1/2 phosphorylation induced by exposure to NT-3, thus indicating that the site of action of MeHg is not on the level of these kinases. FIG. 4(B) shows the level of PDGFR is reduced in the treated cells. The data are consistent with the effects of MeHg being mediated further upstream in the PDGF-signaling pathway. The effects of MeHg on total levels of PDGFRα were prevented by co-exposure with NAC. FIG. 4(C) shows that exposure to MeHg was not associated with reductions in levels of TrkC, indicating that receptor loss was mediated by a mechanism that distinguishes between PDGFRα and TrkC. All experiments were repeated at least three times. The “+” symbol indicates exposure of the cells to the indicated substance.

FIG. 5 shows levels of Fyn kinase activity, levels of c-Cbl phosphorylation, and levels of PDGFR ubiquitylation after MeHg exposure. MeHg exposure caused activation of Fyn, phosphorylation of c-Cbl and ubiquitylation of PDGFRα, leading to reductions in receptor level. FIG. 5(A) shows O-2A/OPCs exposed to 30 nM MeHg exhibited higher levels of Fyn kinase activity, as detected by analysis of immunoprecipitated Fyn from these cells using the Universal Tyrosine Kinase Assay Kit (Takara), as described in Examples. Values (mean±SD) are expressed as the percent of controls, which were defined from basal Fyn kinase activity without any stimulation. All bars with increased levels of Fyn activity differ from control, values at p<0.001. Increased Fyn activity was blocked by pre-treatment of cells with NAC or with the src-family kinase inhibitor PP1, but not by pre-treatment with NH₄Cl (an inhibitor of lysosomal function), FIG. 5(B) shows that, as for the cells in (A), increased c-Cbl phosphorylation (see Examples for immunoprecipitation assay) associated with MeHg exposure was blocked by co-exposure of cells to NAC or PP1, but was not blocked when cells were exposed to NH₄Cl. FIG. 5(C) shows that exposure to MeHg was associated with a marked increase in the levels of ubiquitylation of PDGFRα, with this increase being apparent even though receptor levels were themselves reduced by toxicant exposure. NH₄Cl treatment was associated with rescue of levels of total PDGFRα; and therefore was associated with a still more marked increase in the amount of ubquirylated receptor detected. Co-exposure of cells to PP1 rescued receptor levels and greatly reduced the extent of receptor ubiquitylation. The “+” symbol indicates exposure of the cells to the indicated substance. IB=immunoblot; IP=immunoprecipitation

FIG. 6 shows that MeHg-induced reductions in PDGFRα levels were-prevented by expression of DN(70Z) c-Cbl and by expression of Fyn-specific RNAi constructs. FIG. 6(A) shows that expression (by transfection, as described in Examples) of DN(70Z) c-Cbl prevented MeHg-induced reductions in levels of PDGFRα an effect not obtained with vector alone (pBabe). FIG. 6(B) shows expression of Fyn-specific RNAi (as described in Examples) caused a reduction in levels of Fyn protein, whereas scrambled (Scr) controls had no effect on levels of this protein. Data are presented as comparisons with levels of Fyn protein in non-manipulated cells. FIG. 6(C) shows expression of Fyn-RNAi constructs, but not of Fyn-Scr-RNAi protected O-2A/OPCs from MeHg-induced reductions in levels of total PDGFRα. Fyn RNAi constructs had no effects on levels of tubulin or on levels of c-Cbl. All experiments were repeated at least three times. The “+” symbol indicates exposure of the cells to the indicated substance.

FIG. 7 shows that inhibition of Fyn activity, c-Cbl Activity, or of lysosomal function rescues cell division and/or Erk1/2 phosphorylation in MeHg-treated O-2A/OPCs FIG. 7(A) shows clonal analysis for purified O-2A/OPCs plated at clonal density and analyzed as for FIG. 2. After 24 h, MeHg was added to cultures in the presence or absence of PP1. MeHg by itself was associated with a reduction in the contribution of large clones dominated by progenitors and an increased representation of smaller clones and of oligodendrocytes. Co-exposure to PP1 rescued cells from the effects of MeHg. FIG. 7(B) shows expression of DN(70Z) c-Cbl rescued purified progenitors from MeHg-associated suppression of BrdU incorporation. Expression of DN(70Z) c-Cbl also rescued cells from the effects of 50 nM MeHg. A single asterisk (*) indicates p<0.05; double asterisks (**) indicate=p<0.01. FIG. 7(C) shows co-exposure of cells to NH₄Cl or PP1 together with MeHg protected cells from MeHg-induced suppression of Erk1/2 and PDGFRα phosphorylation and reductions in total levels of PDGFRα. FIG. 7(D) shows overexpression of PDGFRα also rescued cells from MeHg-induced suppression of Erk1/2 phosphorylation, whereas expression of a control construct (pBP) did not rescue Erk1/2 phosphorylation. All experiments were repeated at least three times, and all numerical values represent means±SD for triplicate data points. The “+” symbol indicates exposure of the cells to the indicated substance.

FIG. 8 shows Pb and paraquat exposure caused activation of Fyn and c-Cbl, suppression of Erk1/2 phosphorylation, and reduction in levels of PDGFRα. FIG. 8(A) shows Fyn kinase activity for purified O-2A/OPCs treated as for analysis of MeHg, except that cells were exposed to 1 μM Pb or 5 μM paraquat. Both toxicants caused activation of Fyn, analyzed as in FIG. 5. FIG. 8(B) shows Pb and paraquat exposure also caused phosphorylation of c-Cbl, as detected by immunoprecipitation of total c-Cbl followed by analysis with anti-phosphotyrosine antibody. FIG. 8(C) shows Pb and paraquat exposure caused suppression of Erk1/2 phosphorylation and reductions in total levels of PDGFRα. FIG. 8(D) shows expression of c-Cbl RNAi caused a reduction in levels of c-Cbl protein and protected PDGFRα levels from effects of MeHg, Pb and paraquat, whereas scrambled (Scr) RNAi constructs had no effect on the levels of PDGFRα NAC (or procysteine, a cysteine pro-drug with no intrinsic anti-oxidant activity) protected against all effects of toxicant exposure. All experiments were repeated at least three times. The “+” symbol indicates exposure of the cells to the indicated substance.

FIG. 9 shows exposure of O-2A/OPCs to 1 mM NAC had only minimal effects on basal activity of Fyn or phosphorylation of c-Cbl. Assays of Fyn activity and c-Cbl phosphorylation were carried out as in FIG. 5, except that cells were exposed only to NAC and not to MeHg. FIG. 9(A) shows basal Fyn activity is slightly, but not significantly, lower in cells exposed to NAC. FIG. 9(B) shows the extent of c-Cbl phosphorylation in O-2A/OPCs exposed to NAC is similar to that seen in cells grown in the presence of PDGF only. IB=immunoblot; IP=immunoprecipitation.

FIG. 10 shows exposure to MeHg, Pb, and paraquat caused reductions in levels of c-Cbl targets c-Met and EGFR, but not of TrkC. Cells were analyzed as for FIG. 5, but with antibodies against c-Met and EGFR. The results for the c-Cbl Targets c-Met (A), EGFR (B), and TrkC (C) are shown. All experiments were repeated at least 3 times.

FIG. 11 shows in vivo data confirming that developmental exposure to low levels of MeHg is associated with specific reductions in levels of PDGFRα and EGFR, but not of TrkC, and with reductions in O-2A/OPC division. Treatment of SJL mice with 100 or 250 ppb MeHg in the maternal drinking water during gestation and suckling was associated with reductions in levels of PDGPRα in the cerebellum and hippocampus, at postnatal day 7 (P7), and in hippocampus and corpus callosum at P21. In contrast, levels of the NT-3 receptor TrkC (which is not a c-Cbl target) were not reduced in these animals. FIG. 11(A) shows the results for animals that were treated with 100 ppb MeHg in the drinking water during pregnancy. Pups were sacrificed at 7 d after birth. Analysis of cerebellum and hippocampus showed clear reductions in levels of PDGPRα, but not in TrkC. FIG. 11(B) shows marked reduction in levels of PDGFRα, but not TrkC at P21, in corpus caliosum tissue. FIG. 11(C) shows a quantitative analysis of receptor levels in P7 mice with reductions in levels of PDGFRα and EGFR, but not TrkC. Qualitative analysis of changes in receptor expression is shown for tissue from P7 mice. A single asterisk (*) indicates p<0.05; double asterisks (**) indicate=p<0.01). FIG. 11(D) shows an analysis of BrdU incorporation in Olig2+ cells, which reveals a reduction of approximately 20% in the number of double-positive cells in P14 animals born to mothers receiving 100 ppb MeHg in their drinking water beginning 30 d prior to conception and continuing through weaning. The left photomicrograph shows combined labeling with anti-BrdU and anti-MBP antibodies, and the middle photomicrograph shows labeling with anti-olig2 antibodies. These images are merged in the right photomicrograph to identify BrdU+/Olig2+ cells. Quantitative analysis of total numbers of double-positive cells reveals that developmental exposure to 100 ppb MeHg via the maternal drinking water is associated with a subtle but significant reduction in the number of O-2A/OPCs engaged in DNA synthesis, consistent with the effects of low-level MeHg exposure in vitro. Quantitative data are presented as mean percentage normalized to control animals (n=3 for each group). Error bars represent ±S.E.M. The “+” symbol indicates exposure of the cells to the indicated substance.

FIG. 12 shows a diagrammatic summary of the Fyn/c-Cbl toxicant convergence. The diagram shows a regulatory network in which oxidation causes activation of Fyn. Fyn then phosphorylates c-Cbl Activation of c-Cbl leads to ubiquitylation of agonist-activated RTKs that are c-Cbl targets, with PDGFRα used here as an example of such a receptor. Reductions in levels of receptor lead to reduced activation of downstream signaling cascades.

FIG. 13 shows that MeHg-induced reductions in levels of PDGFRα not dependent; upon changes in transcription of PDGFRα mRNA and exceed normal levels of receptor loss in untreated cells. Cells were grown as for FIG. 5. FIG. 5A shows that despite the reduction in levels of PDGFRα associated with MeHg exposure, this toxicant had no apparent effect on levels of PDGFRα mRNA, as detected by quantitative PCR analysis. FIG. 5B shows that inhibition of protein synthesis with cycloheximide (CHX) was associated with reductions in levels of PDGFRα, but the level of receptor loss occurring when MeHg was also present was markedly more severe. All results are as predicted by the hypothesis that activated c-Cbl enhances degradation of activated PDGFRα but that it is only the degradation step that is lysosome dependent. All experiments were repeated at least 3 times.

FIG. 14 shows that the effects of MeHg, Pb and paraquat were not overridden by bisindoleylmaleimide I (BIM-1), a broad-spectrum PKC inhibitor. O-2A/OPCs were exposed to MeHg (20 nM)s Pb (1 μM) or paraquat (5 μM) for 24 hr with the presence of 0.5 μM of Bisindolymaleimide 1 (BIM-1) or 0.5 μM of PP1. FIG. 14A shows that toxicant-induced increases in Fyn activity were prevented by co-exposure to PP1 but not to BIM-1 FIG. 14B shows that BIM-1 co-exposure did not protect against Pb or paraquat-induced increases in c-Cbl phosphorylation. FIG. 14C shows that toxicant-induced reductions in levels of PDGFRα were prevented by co-exposure of cells to PP1, but not by co-exposure to BIM-1. All experiments were repeated at least 3 times, and all numerical values represent means ±SD for triplicate data points. (*=p<0.05; **=p<0.01).

FIG. 15 shows that analysis with Leadmium™ Green AM demonstrates that NAC did not reduce the levels of Pb in O-2A/OPCs. Cells were incubated in the presence of 1 μM Pb, 1 mM NAC, both together, or neither as for FIG. 8. Over five separate experiments, we found no significant difference between O-2A/OPCs treated with 1 μM Pb vs. [Pb+NAC] (un-paired T-test), and the values for both Pb treated samples were several-fold higher than control values.

FIG. 16 shows that inhibition of Rho kinase does not alter MeHg-associated suppression of BrdU incorporation. Cells were treated as for analysis of effects of MeHg on PDGF-mediated signaling. FIG. 16A shows that the addition of Rho kinase inhibitor Y27632 did not alter the effects of MeHg on O-2A/OPC proliferation. FIG. 16B shows that the concentration of Y27632 used successfully inhibited Rho kinase activity.

FIG. 17 demonstrates that exposure of O-2A/OPCs to even very low levels (100 nM) of tamoxifen activates both Fyn and c-Cbl, and causes reductions in levels of PDGFRα The effects of TMX on activation of Fyn and c-Cbl, and on reductions in levels of PDGFRα, were blocked by NAC and by PP1. In addition, directly blocking the actions of tamoxifen on its receptor with ICI 182780 also prevented Fyn and c-Cbl activation and rescued levels of PDGFRα.

FIG. 18 demonstrates the parallel between the falls in levels of activated PDGFRα (Phospho PDGFR) and levels in total PDGFRα that occur following exposure to tamoxifen. B-tubulin was used as a control to show that the effect on PDGFRα is specific.

FIG. 19 shows that exposure to TMX causes reductions in levels of EGFR and c-Met, as well as of PDGFRα. In contrast, TMX exposure has no effects on levels of TrkC, which is consistent with the outcome of studies on toxicants and by observations that TrkC is not regulated by c-Cbl.

FIG. 20 demonstrates that the inhibition of cell division caused by tamoxifen exposure can be attenuated by MAC, PP1, and ICI 182,780. (Data represent Mean±S.E.M., normalized to control, N=4; *, P<0.05; **, p<0.01.

FIG. 21 shows the percentage of O-2A/OPC cells at various concentrations of TMX, using cells with wild type c-Cbl and cells stably expressing the dominant negative (70Z) mutant of c-Cbl. The percentage of dividing O-2A/OPCs was determined by BrdU incorporation analysis combined with A2B5 staining. Data represent Mean ±S.E.M., normalized to control (N=4). P value was determined by comparing normal and 70z-c-Cbl-expressing O-2A/OPCs exposed to the same concentration of tamoxifen, *, P<0.05; **, p<0.01.

FIG. 22 shows the level of cell survival and proliferation following tamoxifen exposure when NAC and PP1 are administered concommitantly. FIG. 22A and B show cancer cell viability analyzed using MTT assays. Cells cultured in phenol red-tree growth media, were exposed to various concentrations of tamoxifen, with or without NAC. Data represent Mean ±S.E.M (N=6), normalized to control. FIG. 22C shows the level of MDA-MB-231 cell division, analyzed by BrdU analysis, for cells exposed to 20 μM tamoxifen for 48 hours. Neither NAC (1 mM) nor PP1 (200 nM) resulted in any rescue effect. Percentage of BrdU+ cells from DAPI+ cells was determined in each sample. Data represent Mean±S.E.M (N=3).

FIG. 23 shows Western blot analysis demonstrating that agents that induce differentiation of O-2A/OPCs into oligodendrocytes reduce activity along pathways activated by PDGF exposure. PDGF-mediated signaling appears to be altered by TH, BMP, NT-3, FGF-2 at multiple points in the signaling pathway, as in the early stages of our studies on the action of toxicants. Effects of PDGF in the nucleus were examined with reporter constructs for the serum response element (SRE) and for Nf-κB. Further upstream analysis examined phosphorylation of Erk1/2. Exposure to TH or BMP-4 greatly reduced SRE-reporter gene expression. Furthermore, these agents reduce Erk1/2 phosphorylation caused by PDGF, while co-exposure to NT-3 or FGF-2 enhanced signaling. In contrast with the effects on PDGF-mediated signaling, NT-3-stimulated Erk1/2 phosphorylation was not affected by co-exposure to TH, BMP-4, or FGF-2.

FIG. 24 shows TH and BMP exposure is associated with decreases in PDGFRα and c-Met levels, FGF and NT-3 exposure with increased PDGFRα and c-Met levels, and that none of these agents cause changes in levels of TrkC. TH, BMP, NT-3and FGF-2 all converge on regulation of the fyn-c-Cbl pathway, thus exposure to these cell signaling molecules is associated with changes in levels of PDGFRα, with differentiation inducers activating fyn and c-Cbl, leading to reductions in absolute levels of PDGFRα, and enhancers of self-renewal having opposite effects. O-2A/OPCs induced to divide by PDGF to TH or BMP showed reductions in absolute levels of PDGFRα. In contrast, cells grown in the presence of PDGF and exposed to FGF-2 or NT-3 showed an increase in absolute levels of PDGFRα. Furthermore, c-met (another c-Cbl target expressed by O-2A/OPCs) is altered similarly to PDGFRα. Levels of TrkC (the receptor tyrosine kinase for NT-3, and which is not a c-Cbl target) was unaffected by co-exposure of O-2A/OPCs to TH, BMP, FGF-2 or NT-3.

FIG. 25 shows Western blot analysis using a c-Cbl antibody, O-2A/OPCs contacted with PDGF and either TH, BMP-4, FGF-2 and NT-3 showed modified c-Cbl phosphorylation TH and BMP exposure was associated with increased phosphorylation of c-Cbl (See FIG. 25A). FGF-2 or NT-3, however, caused a reduction in c-Cbl phosphorylation (FIG. 25B).

FIG. 26 shows Western blots using PDGFRα antibody with rat )-2A/OPCs grown in the presence of PDGF, FGF-2, and actinomycin D. The membranes were de-probed and then re-probed with β-tubulin antibody. Cells exposed to TH showed lower levels of PDGFRα as in our other experiments. Furthermore, FGF-induced increases in PDGFRα levels are not dependent on gene transcription, as O-2A/OPCs co-exposed to FGF-2 and PDGF in the presence of actinomycin D still showed increased levels of PDGFRα.

FIG. 27 shows Western blots of PDGF-induced phosphorylation of Erk1/2 and of Akt in )-2A/OPCs exposed to EtOH (5% for 16 hours). Reductions in phosphorylation of both Erk1/2 and Akt was prevented when cells were exposed to 1 mM NAC simultaneously with EtOH exposure.

FIG. 28 shows exposure of O-2A/OPCs to EtOH is associated with activation of Fyn and c-Cbl. FIG. 28A shows a Western blot analysis of Fyn phosphorylation, as detected with a pFyn(416) antibody, following exposure of O-2A/OPCs to EtOH (30 min). The EtOH induced increase in Fyn phosphorylation is blocked by co-exposure to NAC. As shown in FIG. 28B, exposure to EtOH also causes c-Cbl phosphorylation, as detected by immunoprecipitation from lysates of treated O-2A/OPCs, followed by analysis with anti-phosphotyrosine antibody.

FIG. 29 shows exposure to EtOH causes reductions in levels of RTKs that are c-Cbl targets (i.e., PDGFRα, c-Met and EGFR), but not of TrkC (which is not a c-Cbl target). Purified O-2A/OPCs were first grown in the presence of PDGF, then switched to medium containing PDGF, NT-3, HGF or EGF (ligands for PDGFRα, TrkC, c-Met or EGFR, respectively) and either grown in control medium or medium containing 0.5% EtOH for 16 hrs, NAC was added 1 hr before EtOH. β-tubulin levels were used as an internal control.

FIG. 30 shows that expression of dominant-negative c-Cbl blocked the EtOH-induced cell death. O-2A/OPCs infected with retrovirus encoding DN(70Z)-c-Cbl, or empty (puronvycin resistance) virus. Twenty-four hr after infection, the cells were collected by trypsinization and reseeded in the selective media (growth media+200 ng/ml puromycin). The infected cells were allowed to proliferate for two days, and then collected and re-seeded for these experiments. (*=p<0.05; **=p<0.01 for comparison with untreated controls; there is no significant difference between DN-c-Cbl cells in control cultures or exposed to 0.5% EtOH for 72 hrs). Expression of DN-c-Cbl also greatly suppressed background cell death in untreated cultures.

FIG. 31 shows the redox/Fyn/c-Cbl pathway in O-2A-OPCs is activated by exposure to amino acid residues 1-42 of amyloid beta (Aβ) protein (Aβ) (5-20 μM). FIG. 31A shows a Western blot using an antibody against activated c-Fyn, in cells exposed to Aβ. Phosphorylated Fyn increased in the presence of Aβ. The increase was reversed by NAC. FIG. 31B shows that Aβ increased levels of phosphorylated c-Cbl. Increases in phosphorylated. c-Cbl is blocked by PP1 or NAC. Phosphorylation of c-Cbl was not blocked, however, by co-exposure of cells to NH₄Cl, which inhibits lysosomal function (which would be relevant to PDGFRα degradation, but not to activation of c-Cbl.

FIG. 32 shows Western blots using antibodies to phosphorylated Erk or antibodies to PDGFRα in O-2A-OPCs contacted with Aβ (1-42). FIG. 32 A shows the results when the cells are also contacted with PDGF. PDGF-induced signaling caused an increase in phosphorylated Erk, but the PDGF-induced increase was reduced when the cells were also contacted with Aβ. FIG. 32B shows Aβ, however, has no effect on Erk phosphorylation, induced by NT-3. FIG. 32C shows that levels of PDGFRα are reduced by exposure to Aβ. This reduction in PDGFRα levels was blocked by PP1 (which prevents Fyn activation), by NAC (which also prevents Fyn activation, but by modulating intracellular redox state) and by NH₄Cl (which inhibits lysosomal degradation). Total Erk and β-tubulin levels are unchanged.

FIG. 33 shows histograms of PDGFRα EGFR, and TRK-c levels expressed as a percentage of control in SJL mice exposed to MeHg (100 ppb or 250 ppb) during development by administration in the maternal drinking water during gestation and suckling. Levels of PDGFRα and EGFR are reduced in the cerebellum (FIG. 33A) and hippocampus (FIG. 33B), whereas levels of Trk-C show no significant difference than control animals. (* indicates p<0.05; ** indicates p<0.01)

FIG. 34 shows levels of phosphorylated c-Cbl in the hippocampus (FIG. 34A) and of levels of PDGFRα and c-Met in the corpus callosum (FIG. 34B) of SJL mice and CBA mice (which are reduced more than their SJL counterparts). c-Cbl is more highly phosphorylated than in the hippocampus of CBA mice than in the more oxidized SJL mice. There are markedly reduced levels of PDGFRα and c-Met in the corpus callosum of SJL mice as compared with CBA mice.

### DETAILED DESCRIPTION

Described herein are methods and compositions related to a previously unrecognized regulatory pathway, the redox/fyn/c-Cbl pathway. This pathway is the pathway upon which environmentally relevant levels of chemically diverse toxicants and other stimuli converge to affect cellular function and cellular division. The pathway is activated when a stimulus makes a cells more oxidized than a comparable cell in a control state or more oxidized than the same cell in the absence of the stimulus. Any number of stimuli can cause the increase in cellular oxidation status aid thereby activate the redox/Fyn/c-Cbl pathway. Such stimuli include, for example, pro-oxidative toxicants, chemotherapeutics, amyloid β, ethanol and other drugs that are abused, triethyltin, hyperglycemia, pro-inflammatory cytokines, immunostimulatory molecules, inducers of scarring (e.g., transforming growth factor-β), inducers of neurodegenerative changes (e.g., amyloid beta peptide), iron, selenium deficiency, agents that cause anti-oxidant imbalances, genetic polymorphisms or mutations that result in cellular oxidative stress and the like. Cellular oxidation activates Fyn kinase, a Src family member. This first step activates a pathway wherein Fyn activates c-Cbl, a ubiquitin ligase that plays a critical role in modulating degradation of a specific subset of receptor tyrosine kinases (RTKs). c-Cbl activation in turn leads to reductions in levels of c-Cbl targeted RTKs, thus suppressing specific cellular functions. c-Cbl targeted RTKs include, by way of example, platelet-derived growth factor receptor-α (PDGFRα), c-Met, and epidermal growth factor receptor (EGFR), Other c-Cbl targets include those described in Schmidt and Dikie, Nat. Rev. Mol. Cell Biol. 6:907-19 (2005), which is incorporated herein in its entirety at least for its disclosure related to c-Cbl targets.

Provided herein is a method of reducing oxidation in a cell by interrupting the redox/Fyn/c-Cbl pathway. The method comprises the steps of providing a cell, wherein the cell has an activated redox/fyn/c-Cbl pathway or is at risk for activation of the redox/fyn/c-Cbl pathway; and contacting (either in vivo or in vitro) the cell with an agent that selectively interrupts the redox/fyn/c-Cbl pathway, wherein interruption of the redox/fyn/c-Cbl pathway reduces oxidation, or an effect of oxidation, as compared to oxidation or the effect of oxidation in the absence of the agent. Thus, the method provides for inactivation of the redox/Fyn/c-Cbl pathway at any of several points in the pathway. For example, by blocking activation of Fyn, by blocking the interaction of Fyn and c-Cbl, by blocking the activation of c-Cbl by blocking the ubiquitylation effect of c-Cbl on a c-Cbl target, or any combination thereof.

A cell used in the methods taught herein has the components for the redox/Fyn/c-Cbl pathway, including at least Fyn, c-Cbl, and one or more c-Cbl targets. Optionally, the cell is recombinantly modified to express one or more of these components. Generally, cellular homeostasis is marked by a steady-state level of oxidation within a range compatible with normal cellular function; however, stimuli can activate the redox/Fyn/c-Cbl pathway. Thus, as used herein, a cell with an activated redox/Fyn/c-Cbl pathway shows an increase in activation over steady-state or over a control level. A control level can be the level of activation in the same cell prior to or after recovery from a pro-oxidative stimulus, or the control level can be the level in a control cell or population of cells in the absence of a stimulus.

In the methods taught herein, selectively interrupt[-ing, -s, -ion] the redox/Fyn/c-Cbl pathway refers to affecting the redox/Fyn/c-Cbl pathway but not other Fyn pathways or other c-Cbl pathways or minimally affecting other Fyn or c-Cbl pathways. Minimally affecting other Fyn or c-Cbl pathways refers to an effect that is substantially less than the redox/Fyn/c-Cbl pathway effect. Thus, some minor, non-specific effect, for example, at high doses of the agent, does not exclude selective interruption of the redox/Fyn/c-Cbl so long as the redox/Fyn/c-Cbl effect is substantially greater (e.g., at least about 50%, 60%, 70%, 80%, 90%, or 100% greater) than the non-specific effect at a selected dosage. By interrupting the redox/Fvn/c-Cbl pathway is meant blocking or inhibiting Fyn activation, blocking or inhibiting Fyn-c-Cbl interaction, or blocking or inhibiting c-Cbl interaction with a c-Cbl target. Thus, blocking does not necessarily refer to a complete elimination of the effect. Blocking c-Cbl interaction with a c-Cbl target, for example, may result in a reduction of about 5%, 10%, 15%, 20%, 25%, or greater reduction in the interaction as compared to a control in the absence of the agent that blocks the c-Cbl interaction. The effect of the interaction can be assessed, for example, by determining the level of ubiquitylation of the c-Cbl target or may involve measure the level and/or activation of a downstream cellular product affected by the c-Cbl target.

An agent that selectively interrupts the redox/Fyn/c-Cbl pathway optionally reduces activation of Fyn kinase, interaction of Fyn kinase and c-Cbl ubiquitin ligase, c-Cbl ubiquitin ligase activation, or reduces c-Cbl ubiquitin ligase interaction with a target, as compared to activation of Fyn kinase, interaction of Fyn kinase and c-Cbl ubiquitin ligase, c-Cbl ubiquitin ligase activation, or interaction of c-Cbl ubiquitin ligase interaction in a control. Examples of agents that selectively interrupt the redox/Fyn/c-Cbl pathway include antisense or interfering nucleic acids. More specifically, siRNAs can be designed to inhibit a Fyn kinase or a c-Cbl inhibitor. Examples of such siRNAs can be found in Table 1 as SEQ ID NOs:1-8. Further provided are siRNAs comprising the nucleotide sequences of any one SEQ ID NOs:1-8 with insertions, deletions, substitutions, or other selected modifications. Small molecules that selectively interrupt the redox/Fyn/c-Cbl pathways can be developed using rational drug design or by screening libraries of small molecules for an agent that selectively interrupts the pathway, using screening methods as taught herein. In addition, in vitro screening assays may be used to identify agents that suppress the activity of Fyn, the activation of c-Cbl by Fyn, the binding of Fyn to c-Cbl or the binding and/or ubiquitylation by c-Cbl of its target proteins.

Activation of the redox/fyn/c-Cbl pathway in a cell is optionally caused by any one of or any combination of stimuli, including, for example, a chemotherapeutic agent, a toxicant (e.g., MeHg, Pb, paraquat), ethanol, and amyloid β. Thus, risk for activation of the redox/Fyn/c-Cbl pathway can be related to such events as exposure to a toxicant, a chemotherapeutic agent, a pathogen, or other stimulus that increases cellular oxidation and activates the redox/Fyn/c-Cbl pathway.

The cell of the methods taught herein is optionally a precursor cell. By precursor cell is meant a stem cell (either a tot potent or multipotent stem cell) or a lineage committed progenitor cell, including, for example, a neural progenitor or a pancreatic islet cell progenitor. The neural progenitor is optionally an oligodendrocyte/type-2 astrocyte progenitor, also known as an oligodendrocyte progenitor and referred to herein as either a O-2A cell or a O-2A/OPC. Alternatively the cell of the methods taught herein is differentiated cell, such as, by way of example only, a neuron or an insulin secreting cell, such as a pancreatic islet β cell.

Because the redox/Fyn/c-Cbl pathway is a point of convergence for various toxicants, the pathway can be utilized to screen for a pro-oxidation toxicant. Thus provided herein is a method of screening for a pro-oxidation toxicant comprising the steps of contacting a precursor cell with an agent to be tested for pro-oxidative toxic effects; and detecting activation, of a redox/fyn/c-Cbl pathway. Activation of the redox/Fyn/c-Cbl pathway indicates the agent has pro-oxidation toxic effects. Activation of the redox/Fyn/c-Cbl pathway can be assessed at any point in the pathway, including, for example, by measuring Fyn kinase activation, c-Cbl ubiquitin ligase activation, or of the level of one or more c-Cbl targets. More specifically, downregulation of one or more selective targets of c-Cbl ubiquitin ligase would indicate an agent that selectively interrupts the redox/Fyn/c-Cbl pathway. Selective targets of c-Cbl include, for example, PDGFR. EGFR, c-Met and other receptor tyrosine kinases. To verify selectivity of the agent that interrupts the redox/Fyn/c-Cbl pathway, the method optionally further comprises detecting the absence of downregulation of a non-c-Cbl ubiquitin ligase target. An example of a non-c-Cbl ubiquitin ligase target is TrkC.

O-2A/OPCs offer several advantages for use in the method of screening toxicants. These cells are sensitive to environmentally relevant exposure levels of toxicants and they have been well characterized. They can also be used in clonal analysis in quantitative studies on the cumulative effects of small changes in the balance between division and differentiation. However, Fyn and e-Cbl are expressed in other cells types, which may also be used in the methods as described herein. Thus, any cell type expressing Fyn and c-Cbl may be used in the screening methods described herein.

Also provided herein is a method of determining a concentration of a toxicant that has deleterious pro-oxidative effects. The method includes the steps of contacting one or more precursor cells, in vivo or in vitro, with one or more concentrations of the toxicant to be tested; and detecting the level of activation of a redox/fyn/c-Cbl pathway at each concentration. An activation at a given concentration(s) above control levels indicates the concentration(s) has pro-oxidative effects. The determination of the concentration of the toxicant that has pro-oxidative effects can be used to determine what levels of the toxicant can be tolerated in the environment by adult or juvenile subjects.

Also provided herein are methods of expanding populations of precursor cells by interrupting the redox/Fyn/c-Cbl pathway. Several general classes of agents are relevant to understanding how the balance between division and differentiation is controlled in dividing precursor cells. Some agents act as mitogens that support limited proliferation in vitro. In general, exposure to a single mitogen (usually in association with exposure to insulin and transferrin) fails to confer the capacity for extended self-renewal. A second class of agents are differentiation agents, which suppress division and promote cellular differentiation. A third class of agents are those which work oppositely to differentiation inducers. Agents in this third class, when combined with mitogens, will suppress differentiation and enable precursor cells to undergo continued proliferation.

The ability to promote continued proliferation in the absence of differentiation is critical for expanding precursor cell populations ex vivo for use in, e.g., subsequent cell transplantations for tissue repair. The ability to promote continued proliferation is also of great importance in promoting wound healing in vivo, by enabling increased numbers of precursor cells to be generated for the purpose of tissue repair. While there has been significant interest in the identification of cellular mechanisms that regulate this process, no critical pathway was previously understood that regulated the balance between differentiation and proliferation. Thus, provided herein is a method of promoting proliferation of a precursor cell comprising contacting the precursor cell with an agent that selectively interrupts the redox/fyn/c-Cbl pathway. The precursor cell can be a stem cell (including pluripotent cells such as an ES cell or multipotent cells like a hematopoietic cell) or can be a lineage restricted or committed cell such as a neural progenitor cell or a progenitor of a insulin producing cell.

A method of promoting differentiation of a progenitor cell or its progeny comprising contacting the O-2A cell with an agent that selectively activates the redox/fyn/c-Cbl pathway is also provided.

Interrupting the redox/Fyn/c-Cbl pathway is also useful in certain disease states or treatment paradigms. Because chemotherapeutics agents activate the redox/Fyn/c-Cbl pathway in normal cells but not in cancer cells, selective chemoprotection can be provided to non-cancer cells by interrupting the redox/Fyn/c-Cbl pathway. More specifically, provided herein is a method of treating a subject with cancer comprising the steps of administering to the subject a chemotherapeutic agent, and administering to the subject an agent that selectively interrupts the redox/fyn/c-Cbl pathway, thereby selectively providing chemoprotection to the healthy, non-cancer cells. Chemotherapeutic agents are well known in the art and include, by way of example, cisplatin, camiustine, cytarabine, 5-fluorouracil and tamoxifen.

Provided herein is a method of detecting an oxidized state in a test cell comprising detecting the level of one or more c-Cbl targets and the level of one or more non-Cbl targets. A reduced level of one or more c-Cbl targets, as compared to a control cell, in the absence of a reduced level of the non-Cbl target indicates the oxidized state in the test cell. This method can be practiced using a biological sample from a subject. Thus, further provided is a method of testing oxidation levels in a subject comprising detecting the level of one or more c-Cbl targets in a biological sample from the subject and detecting the level of one or more non-Cbl targets in the biological sample. A reduced level of one or more c-Cbl targets, as compared to control levels, in the absence of a reduced level of the non-Cbl target indicates an oxidized state and c-Cbl activation in the subject. The biological sample can be any tissue or cellular sample (including, for example, a tissue biopsy, blood cells, marrow cells, muscle cell, skin cells, or the like) or fluid sample (including, for example, a blood, sputum, plasma, synovial fluid, tear) or other biological sample.

Individual subjects who are more oxidized (i.e., have cells with higher oxidation levels than control levels of oxidation) are thought to be more vulnerable to the toxic effects of physiological stressors. Thus, individuals with highly oxidized states are at risk for a variety of conditions and diseases. For example, cells from children with autism are more oxidized than those from non-autistic children. Such a status makes these children more vulnerable to the effects of exposure to environmental toxicants, to traumatic tissue damage or to the effects of inflammation because of the impact of these pro-oxidative stimuli on the redox/Fyn/c-Cbl pathway. The methods taught herein are useful to identify a subject at risk for a highly oxidized state, a subject is at risk for autism, a subject at risk for Alzheimer's disease, a subject is at risk for effects from exposure to environmental toxicants, and the like.

Furthermore, individuals with highly oxidized states could show greater sensitivity to certain treatments, including, for example, chemotherapeutics or radiation. Thus, the methods of detecting the oxidation, state in a subject can be used to select treatment or to monitor the oxidative effect of treatment. Following exposure to a toxicant or suspected exposure to a toxicant, levels of oxidation can be monitored to determine if and when an agent that interrupts the redox/Fyn/c-Cbl pathway should be administered. Thus, any of the methods of testing oxidation levels in a subject can further comprise selecting a treatment for the subject based on the subject's oxidation level.

Also provided herein is a method of screening for an agent that selectively interrupts the redox/fyn/c-Cbl pathway comprising providing a cell wherein the cell has an activated redox/fyn/c-Cbl pathway; contacting the cell with the agent to be screened; and detecting one or more parameters of an interrupted redox/Fyn/c-Cbl pathway. Such parameters include, for example, reduced Fyn activation, reduced Fyn/c-Cbl interaction, reduced c-Cbl activation, or reduced levels of c-Cbl targets.

Provided herein are agents that selectively interrupt the redox/fyn/c-Cbl pathway. Such agents include, for example, small molecules, siRNAs, antisense oligonucleotides, and peptides, antibodies. Thus, provided herein is an agent identified by the screening methods taught herein. The agents, whether isolated from a biological sample, identified by a screening method taught herein, recombinantly expressed, or synthesized include for example, small molecules, anti-sense oligonucleotide, or an interfering RNA. Optionally, the agent is an isolated small inhibitory ribonucleic acid that inhibits expression of a nucleic acid encoding either c-Cbl or Fyn. Examples of siRNAs include those having the nucleic acid sequence of any of SEQ ID NOs:1-8. Optionally, the RNA is double stranded. Thus, provided herein is an isolated double stranded RNA comprising a first strand of nucleotides substantially identical to any one of the nucleotide sequences of SEQ ID NOs:1-8 and a second strand substantially complementary to the first. Also provided is an isolated double stranded RNA comprising a first strand of nucleotides as set forth in any one of the nucleotide sequences of SEQ ID NOs:1-8 and a second strand substantially complementary to the first. Any of the RNAs taught herein and others can be designed to inhibit expression of either c-Cbl or Fyn. Other RNAs can be designed which target transcription factors that affect c-Cbl or Fyn. Optionally, the double stranded RNAs as taught herein have at one or both ends an overhang of 1 to 4 nucleotides.

siRNAs can be synthesized and annealed prior to delivery to the cell or subject. The oligonucleotide can be chemically synthesized or can be produced using recombinant DNA methods, using an appropriate template. Oligonucleotides comprising nucleotide analogs or covalent bonds other than phosphodiester bonds generally will be chemically synthesized, although an enzyme such as T7 polymerase can incorporate certain types of nucleotide analogs into a polynucleotide and therefore, can be used to produce such a polynucleotide recombinantly from an appropriate template (Jellinek et al., Biochemistry 34:11363-11372 (1995)), Thus, the oligonucleotides can be made using standard chemical synthesis methods or can be produced using enzymatic methods or any other known method. Such methods can range from standard enzymatic digestion followed by nucleotide fragment isolation (see for example, Sambrook et al.) to purely synthetic methods, for example, by the cyanoethyl phosphoramidite method using a Milligen or Beckman System IPlus DNA synthesizer (for example, Model 8700 automated synthesizer of Milligen-Biosearch, Burlington, Mass. or ABl Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

Alternatively, the siRNA can be synthesized in vivo, such as from a plasmid expression system. See, e.g., Tuschl and Borkhardt, Molec. Interventions 2:158-167, 2002, for review. The siRNAs can be delivered into cells by electroporation or using lipophilic reagents. The siRNAs can be administered to subjects, for example, by intravenous injection, direct injection into a target site, and the like, as described in more detail below.

For description of making and using RNAi molecules see, e.g., Hammond et al., Nature Rev Gen 2; 110-119 (2001); Sharp, Genes Dev 15: 485-490 (2001); Waterhouse et al., Proc. Natl. Acad. Sci. USA 95(23); 13959-13964 (1998) all of which are incorporated herein by reference in their entireties and at least form material related to delivery and making of RNAi molecules. Synthetic siRNAs are generally designed using algorithms and a conventional DNA/RNA synthesizer. Suppliers include Ambion (Austin, Tex.), ChemGenes (Ashland, Mass.), Dharmacon (Lafayette, Colo.), Glen Research (Sterling, Va.), MWB Biotech. (Esbersberg, Germany), Proligo (Boulder, Colo.), and Qiagen (Vento, The Netherlands). siRNA can also be synthesized in vitro using kits such as Amnion's SILENCER siRNA Construction Kit. Disclosed herein are any siRNA designed as described above based on the sequences for Fyn or c-Cbl.

Also, provided herein are constructs used for making double stranded RNAs, as described, for example, in U.S. Pat No, 6,573,099, which is incorporated herein in its entirety at least for the constructs and methods of making and using the constructs. Thus, provided herein is an isolated nucleic acid comprising an expression control sequence operatively linked to a nucleotide sequence that is a template for one or both strands of the dsRNA taught herein. Optionally, a single transcription unit of the construct can generate, in a single RNA transcript, both a sequence identical to or substantially identical to the target RNA sequence and its complement, with the two sequences separated by a loop region, to form a hairpin structure. Thus provided herein is an isolated nucleic acid comprising an expression control sequence operatively linked to a transcription unit encoding an RNA transcript that comprises the first and second strands of a double stranded RNA as taught herein, wherein the first strand is located at the 5′ end of the unit and the second strand is located at the 3′ end of the unit, wherein the first and second strands are separated by about 3 to about 500 base pairs and wherein the RNA transcript, after transcription, forms a hairpin.

Cells comprising the constructs for making the double stranded RNAs are provided. Also provided are compositions comprising the agent that selectively interrupts the redox/Fyn/c-Cbl pathway and a carrier. Such a composition comprises the isolated double stranded RNA in an amount sufficient to inhibit expression of either c-Cbl or Fyn.

Nucleic acids, preferably with nucleotide residues numbering 5, 10, 20, 30, 40, 50, 60, 70, 80, 90, 100, 200, 300, or any amount in between, that hybridize under stringent conditions to any one of the nucleotide sequences of SEQ ID NOs.1-8 or their complements are taught. Nucleic acid duplex or hybrid stability is expressed as the melting temperature or Tm, which is the temperature at which a probe dissociates from a target DNA. This melting temperature is used to define the required stringency conditions. If sequences are to be identified that are related and substantially identical to the probe, rather than identical, then it is useful to first establish the lowest temperature at which only homologous hybridization occurs with a particular concentration of salt. Then, assuming a 1% mismatch results in a 1° C. decrease in the Tm, the temperature of the final wash in the hybridization reaction is reduced accordingly. For example, if the sequences having 95% identity with the probe are desired, the final wash temperature is decreased by 5° C. In practice the change in Tm can be between 0.5 and 1.5° C. per 1% mismatch. Stringent conditions involve hybridizing at 68° C. in 5×SSC/5×Denhardt's solution/1.0% SDS, and washing in 0.2×SSC/0.1% SDS at room temperature. The parameters can be varied to achieve the optimal level of identity between the probe and the target nucleic acid. Additional guidance can be round, for example, in Sambrook et al., Molecular Cloning: A Laboratory Manual, Cold Spring Harbor Press, New York.

Antibodies that selectively interrupt the redox/Fyn/c-Cbl pathways are provided. Such antibodies optionally block Fyn or c-Cbl function, by binding to active sites of Fyn, c-Cbl or related molecules. Such antibodies can be made using techniques well known in the art. Blocking functions of the antibodies can be assessed by contacting a cell or administering to a subject the antibody and assaying the activity of the redox/Fyn/c-Cbl pathway, as described in the Examples. Optionally such antibodies are humanized or are fully human to reduce immunogenicity caused by in vivo administration. Methods for humanizing non-human antibodies or making fully human antibodies are well known in the art. Generally, a humanized antibody has one or more amino acid residues introduced into it from a source that is non-human. These non-human amino acid residues are often referred to as import residues, which are typically taken from an import variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species, in practice, humanized antibodies are typically human antibodies in which some CDR residues and possibly some FR residues are substituted by residues from analogous sites in rodent antibodies. Transgenic animals (e.g., mice) that are capable, upon immunization, of producing a full repertoire of human antibodies in the absence of endogenous immunoglobulin production can be employed to make fully human antibodies. For example, it has been described that the homozygous deletion of the antibody heavy chain joining region (J(H)) gene in chimeric and germ-line mutant mice results in complete inhibition of endogenous antibody production. Transfer of the human germ-line immunoglobulin gene array in such germ-line mutant mice will result in the production of human, antibodies upon antigen challenge (see, e.g., Jakobovits et al., Proc. Natl, Acad. Sci. USA, 90:2551-255 (1993); Jakobovits et al., Nature, 362:255-258 (1993); Bruggemann et al., Year in Immuno., 7:33 (1993)). Human antibodies can also be produced in phage display libraries (Hoogenboom et al., J. Mol. Biol, 227:381 (1991); Marks et al., J. Mol. Biol., 222:581 (1991)). The techniques of Cote et al., and Boerner et al., are also available for the preparation of human monoclonal antibodies (Cole et al., Monoclonal Antibodies and Cancer Therapy, Alan R. Liss, p. 77 (1985); Boerner et al, J. Immunol, 147(1):86-95 1991)).

Also provided are functional, antibody fragments that interrupt the redox/Fyn-c-Cbl pathway. Such fragments include F(ab′)2, Fab′, Fab and the like.

Similarly, polypeptide fragments of c-Cbl or Fyn that do not activate the downstream pathway can be used as competitive blockers of the redox/Fyn/c-Cbl pathway. Such peptides can be designed and can be synthesized using peptide synthesis techniques or by cleaved from the full-length proteins. If desired or necessary, in order to increase cellular uptake of the peptides, the peptides can be conjugated to moieties or encapsulated, for example, in liposomes or nanoparticles by methods known in the art. For example, the antibodies and peptides described herein can be conjugated to tat peptides derived from HIV-1 tat protein, folate, another antibody that targets a receptor such as the transferring receptor.

Ranges maybe expressed herein as from about one particular value, and/or to about another particular value. When such a range is expressed, another embodiment includes from the one particular value and/or to the other particular value. Similarly, when values are expressed as approximations, by use of the antecedent about, it will be understood that the particular value forms another embodiment. It will be further understood that the endpoints of each of the ranges are significant both in relation to the other endpoint, and independently of the other endpoint.

The compositions and agents that selectively interrupt the redox/Fyn/c-Cbl pathway are optionally administered in vivo in a pharmaceutically acceptable carrier. By pharmaceutically acceptable is meant a material that is not biologically or otherwise undesirable. Thus, the material may be administered to a subject, without causing undesirable biological effects or interacting in a deleterious manner with any of the other components of the pharmaceutical composition in which it is contained. The carrier would naturally be selected to minimize any degradation of the active ingredient and to minimize any adverse side effects in the subject.

The agent or compositions may be administered orally, parenterally (e.g., intravenously), by intramuscular injection, by intraperitoneal injection, transdermally, extracorporeally, topically or the like, including intranasal administration or administration by inhalant. The dosage of the agent or composition required will vary from subject to subject, depending on the species, age, weight and general condition of the subject, the severity of the airway disorder being treated, the particular active agent used, its mode of administration and the like. Thus, it is not possible to specify an exact amount for every composition. However, an appropriate amount can be determined using only routine experimentation given the teachings herein.

Suitable carriers and their formulations are described in Remington: The Science and Practice of Pharmacy (21st ed.) eds. A. R. Gennaro et al., University of the Sciences in Philadelphia 2005. Typically, an appropriate amount of a pharmaceutically-acceptable salt is used in the formulation to render the formulation isotonic. Examples of the pharmaceutically-acceptable carrier include, but are not limited to, saline. Ringer's solution and dextrose solution. The pH of the solution is preferably from about 5 to about 8.5, and more preferably from about 7.8 to about 8.2. Further carriers include sustained release preparations such as semipermeable matrices of solid hydrophobic polymers containing the antibody, which matrices are in the form of shaped articles, e.g., films, liposomes or microparticles. Certain carriers may be more preferable depending upon, for instance, the route of administration and concentration of composition being administered.

Pharmaceutical carriers most typically would be standard carriers for administration of drugs to humans, including solutions such as sterile water, saline, and buffered solutions at physiological pH. Other compounds will be administered according to standard procedures.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The terms effective amount and effective dosage are used interchangeably. The term effective amount is defined as any amount necessary to produce a desired physiologic response. Effective amounts and schedules for administering the compositions may be determined empirically, and making such determinations is within the skill in the art. The dosage ranges for the administration of the compositions are those large enough to produce the desired effect in which the symptoms or disorder are affected. The dosage should not be so large as to cause substantial adverse side effects, such as unwanted cross-reactions, anaphylactic reactions, and the like.

As used throughout, by a subject is meant an individual. Thus, the subject can include domesticated animals, such as cats, dogs, etc., livestock (e.g., cattle, horses, pigs, sheep, goats, etc.), laboratory animals (e.g., mouse, rabbit, rat, guinea pig, etc.) and birds. Preferably, the subject is a mammal such as a primate, and, more preferably, a human.

By isolated nucleic acid or purified nucleic acid is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (e.g., a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, e.g., other types of RNA molecules or polypeptide molecules.

As used herein, references to decreasing, reducing, or inhibiting include a change of 10, 20, 30, 40, 50, 60, 70, 80, 90 percent or greater as compared to a control, level. Such terms can include but do not necessarily include complete elimination.

There are a variety of sequences related to, for example, Fyn and c-Cbl that are disclosed on Genbank, at www.pubmed.gov and these sequences and others are herein incorporated by reference in their entireties as well as for individual subsequences contained therein.

Treatment or treating, as used herein, means to administer a composition to a subject with a condition, wherein the condition can be any pathologic disease, such as cancer or Alzheimer's Disease. The effect of the administration to the subject can have the effect of but is not limited to reducing one or more symptoms of the condition, a reduction in the severity of the condition, the complete ablation of the condition, or a delay in the onset or worsening of one or more symptoms.

Herein, activation or activates means to increase activity. It is understood that activation can mean an increase in existing activity as well as the induction of new activity.

Throughout this application, various publications are referenced. The disclosures of these publications in their entireties are hereby incorporated by reference into this application in order to more fully describe the state of the art to which this invention pertains.

A number of embodiments of the invention have been described. Nevertheless, it will be understood that various modifications may be made without departing from the spirit and scope of the invention. Furthermore, when one characteristic or step is described it can be combined with any other characteristic or step herein even if the combination is not explicitly stated. Accordingly, other embodiments are within the scope of the claims.

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention except as and to the extent that they are included in the accompanying claims. Efforts have been made to ensure accuracy with respect to numbers (e.g., amounts, temperature, etc.), but some errors and deviations should be accounted for.

### EXAMPLES

### Example 1

### Chemically Diverse Toxicants Converge on Fyn and c-Cbl to Disrupt Prescursor Cell Function

The present study shows that chemically diverse toxicants converge on the redox-Fyn/c-Cbl pathway, at environmentally relevant exposure levels, to disrupt the function of progenitor cells of the developing central nervous system. As described below, low levels of methylmercury, lead, and paraquat caused activation of Fyn kinase, which activated c-Cbl, which in turn modified specific proteins, receptors required for cell division and survival. The c-Cbl mediated modification initiated the receptors' degradation, thereby repressing downstream signaling from the receptors. Analysis of developmental exposure to methylmercury showed the same pathway was activated in vivo by environmentally relevant toxicant levels. The remarkable sensitivity of neural progenitor cells to low levels of toxicant exposure, and the discovery of the redox/Fyn/c-Cbl pathway as a mechanism by which small increases in oxidative status can markedly alter cell function, provided a novel and specific means for testing perturbation of normal development caused by exposure to chemically diverse toxicants.

Materials and Methods

Cell Isolation, Culture, and Treatment.

O-2A/OPC-S were purified from corpus callosum of P7 CD rats as described previously to remove type 1 astrocytes, leptomeningeal cells, and oligodendrocytes [9,14,199], Cells were then grown in DMEM/F12 supplemented with 1-μg/.rnl bovine pancreas insulin (Sigma, St. Louis, Mo., United States), 100-μg/ml human transferrin (Sigma), 2 mM glutamine, 25-μg/ml gemamicin, 0.0286% (v/v) BSA pathocyte (ICN Biochemicals, Costa Mesa, Calif., United States), 0.2 μM progesterone (Sigma), 0.10 μM putrescine (Sigma), bFGF-2 (10 ng/ml, PEPRO Technologies, London, United Kingdom), and PDGF-AA (10 ng/ml, PEPRO) onto poly-1-lysine (Sigma) coated flasks or dishes. Under these conditions, O-2A/OPCs derived from the corpus callosum of P7 rats are predominantly in cell division and do not generate large numbers of oligodendrocytes during the time periods utilized in this analysis.

To generate sufficient numbers of cells for biochemical analysis, cells, were expanded through one to two passages in PDGF+FGF-2 before replating in the presence of PDGF alone. When cells achieved approximately 50% confluence, MeHg, Pb, or paraquat was added to their medium at concentrations indicated. Doses for the toxicants were chosen on the basis of dose-response curves to identify sub-lethal exposure levels, as a reflection of blood and brain toxicant levels of these compounds and, where applicable, on the basis of previous reports. All toxicant concentrations examined were confirmed to cause death of less than 5% of cells over the time course of the experiment.

For analysis of the effects of potential inhibitors of toxicant action, cells were exposed to the blocking compound of interest 1 h before addition of toxicant. The concentrations of inhibitors used are listed as the following: 0.5 μM BIM-1 (PKC inhibitor), 0.5 μM PP1/PP2 (Src family kinase inhibitors), and 10 mM NH₄Cl (lysosome inhibitor); and the concentrations of toxicants used are listed as following except when otherwise mentioned specifically: MeHg (20 nM), Pb (1 μM), and paraquat (5 μM).

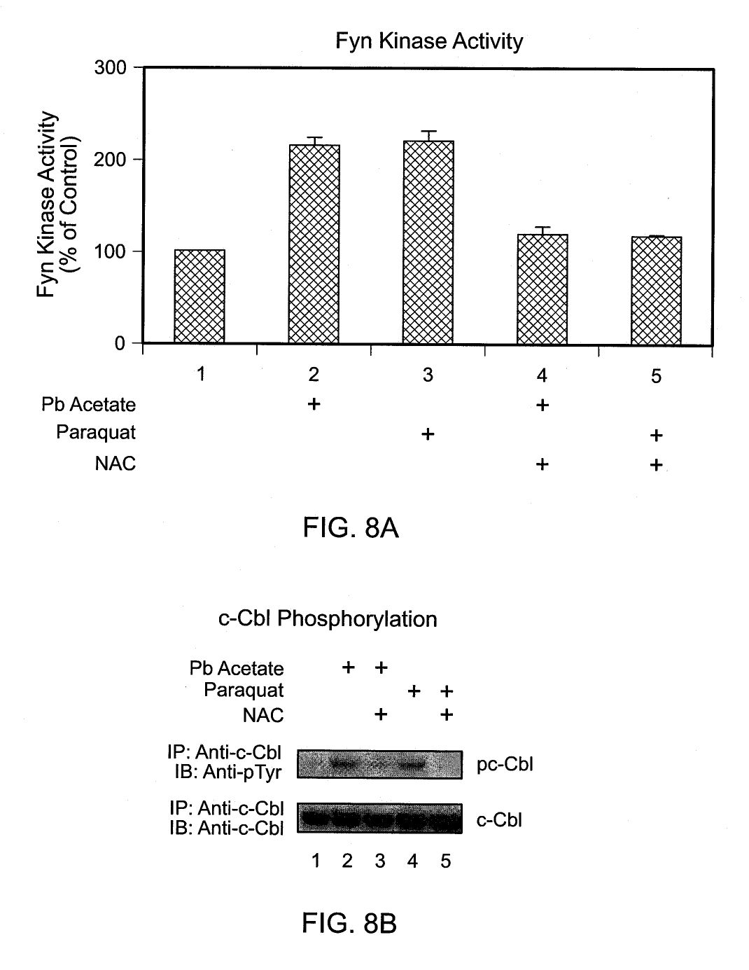

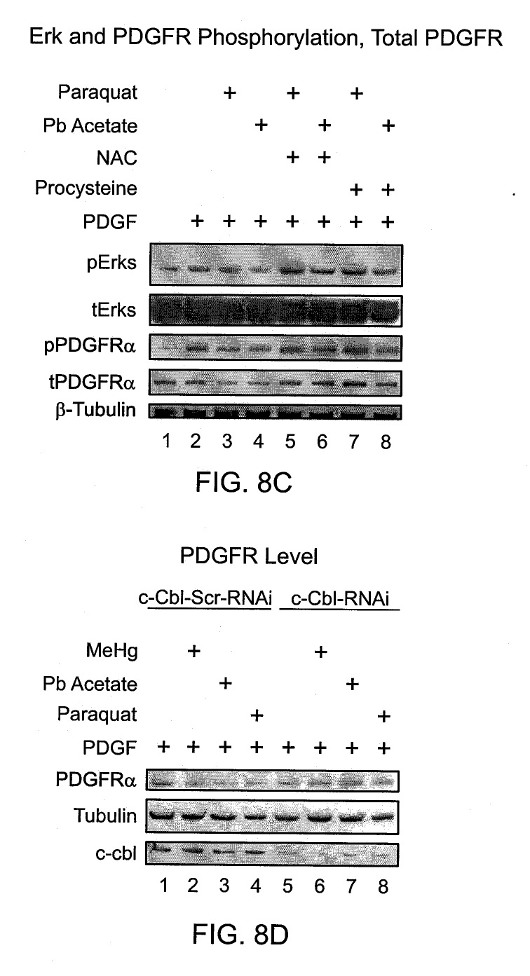

To examine the degradation of PDGFRα, O-2A/OPCs were treated with MeHg (20 nM) for different durations with or without cycloheximide (CHX, 1 μg/ml) added 1 h before MeHg. The cells were then collected and lysed for Western blotting. For example, in the multi-toxicant analysis of FIGS. 8-10, for analysis of PDGFRα; O-2A/OPCs were exposed for 24 h to MeHg (20 nM), Pb (1 μM), and paraquat (5 μM) for 24 h in the presence of 0.5 μM bisindolymaleimide I (BIM-1), 0.5 μM of PP1, 1 mM NAC, or 1 mM procysteine, which had been added 1 h prior to toxicant addition. Cells were lysed for Western blot analysis using anti-PDGFRα(pY742) antibody. The membranes were de-probed and then re-probed with antibody against total PDGFRα and anti-β-tubulin antibody. For analysis of Fyn activity and c-Cbl phosphorylation, progenitors were exposed to MeHg (20 nM), Pb (1 μM), and paraquat (5 μM) for 3-4 h in the presence of 0.5 μM BIM1, 0.5 μM PP1, or 1 mM: NAC (each of which was added 1 h before addition of toxicant).

Cell transfection and Luciferase Activity Assay.

Cells were deprived of PDGF-AA for 5 h before re-exposure to PDGF-AA (10 ng/ml) for 1 h for Western blot or 6 h for luciferase assays of pathway activation. Transient transfection was performed using Fugene6 (Roche, Basel, Switzerland ) transfection solution according to the manufacturer's protocol. For the luciferase assay, cells seeded in 12-well plates were transfected with a reporter plasmid SRE-Luc(firefly) or NF-κB-Luc(firefly) (BD-Clontech, Palo Alto, Calif., United States) and an internal control plasmid pRLSV40-LUC. Analyses of luciferase activity were performed according to the protocol of the Dual Luciferase Assay System (Promega, Madison, Wis., United States), which uses an internal control of Renilla luciferase for quantification, and relative light units were measured using a luminometer.

Antibodies and Immunoblotting.

Anti-phosphorylated Erk monoclonal, anti-Erk monoclonal, anti-TrkC polyclonal anti-Fyn polyclonal, anti-EGFR polyclonal, anti-c-Met polyclonal, anti-phospho-tyrosine monoclonal, and anti-PDGFRα polyclonal antibodies were obtained from Santa Cruz Biotechnology (Santa Cruz, Calif., United States). Anti-c-Cbl monoclonal antibody was obtained from BD PharMingen (San Diego, Calif., United States). Anti-phosphorylated Akt monoclonal and anti-Akt polyclonal antibodies were obtained from Cell Signaling Technology (Beverly, Mass., United States). Anti-phosphorylated PDGPRα polyclonal antibody was obtained from Biosource (Carlsbad, Calif., United States). The cell culture samples were collected and lysed in RAPI buffer, whereas dissected tissue samples were sonicated in RAPI buffer. Samples were resolved on SDS-PAGE gels and transferred to PVDF membranes (PerkinElmer Life Science. Wellesley, Mass., United States), After being blocked in 5% skim milk in PBS containing 0.1% Tween 20, membranes were incubated with a primary antibody, followed by incubation with an HRP-conjugated secondary antibody (Santa Cruz Biotechnology). Membranes were visualized using Western Blotting Luminol Reagent (Santa Cruz Biotechnology). All analyses of signaling pathway components were conducted in the presence of ligand for the receptor pathway under analysis (either PDGF-AA for PDGFRα, NT-3 or TrkC, HGF for c-Met, or EGF for EGFR).

In vitro BrdU Incorporation Assay.

Cell proliferation was assessed by bromodeoxyuridine (BrdU) incorporation and by using the mouse anti-BrdU mAb IgGl (1:100; Sigma) to label dividing cells. Stained cells on cover slips were rinsed two times in 1×PBS, counterstained with 4′6-diamidino-2-phenylindole (DAPI; Molecular Probes, Eugene, Oreg., United States) and mounted on glass slides with Fluoromount (Molecular Probes). Staining against surface proteins was performed on cultures of living cells or on cells fixed with 2% paraformaldehyde. Staining with intracellular antibodies was performed by permeabilizing cells with ice-cold methanol for 4 min or by using 0.5% Triton for 15 mm on 2% parafomaldhyde-fixed cells. Antibody binding was detected with appropriate fluorescent dye-conjugated secondary antibodies at 10 μg/ml (Southern Biotech, Birmingham, Ala., United States) or Alexa Fluor-coupled antibodies at a concentration of 1 μg/ml (Molecular Probes), applied for 20 min. Anti-BrdU monoclonal antibody was obtained from Sigma.

Intracellular ROS Measurement and Analysis of Pb Uptake.

Cells were plated in 96-well microplates and grown to about 60% continence. Prior to treatment, cells were washed twice with Hank's buffered saline solution (HBSS), loaded with 20 μM H2DCFDA (in BBSS 100 μl/well), and incubated at 37° C. for 30 min. Cells were then washed once with HBSS and growth medium to remove free probe. Then, fresh growth medium was added and a baseline fluorescence reading was taken prior to treatment. For NAC pre-treatment. NAC was added into media 1 hour before further addition of MeHg, and both compounds remained in the medium during the incubation period with H2DCFDA. Fluorescence was measured in a Wallac 1420 Victor2 multilabel counter (PerkinElmer) using excitation and emission wavelengths of 485 nm and 535 nm, respectively, at different time courses as indicated in the figures. Results are presented as the value change from baseline by the formula (Ftexp−Ftbase)/Ftbase normalized with the control group, where Ftexp=fluorescence at any given time during the experiment in a give well and Ftbase=baseline fluorescence of the same well.

Pre-treatment with NAC was assayed to determine whether levels of intracellular Pb was altered using analysis with the Leadmium Green AM dye (Molecular Probes), according to the manufacturer's instructions. In five separate experiments, no significant difference was found between O-2A/OPCs treated with 1 μM Pb versus [Pb+NAC] (unpaired t-test), and the values for both Pb-treated samples were several-fold higher than control values. All of these data show that the major effect of NAC is to antagonize cellular oxidation.

Immunoprecipitation Assay.

For the co-immunoprecipitation assay, anti-c-Cbl monoclonal antibody (BD PharMingen) or anti-PDGFRα polyclonal antibody (Santa Cruz Biotechnology) was added to the pre-cleared cell lysates (250 μg of total protein), and the mixtures were gently rocked for 2 h at 4° C. A total of 30 μl of protein A/G agarose was then added to the mixture followed by rotating at 4° C. overnight. The protein A/G agarose was then spun down and washed thoroughly three times. The precipitates were resolved on an 8% SDS-PAGE gel and then were subjected to Western blot analysis using an anti-p-Tyr (for c-Cbl phosphorylation assay) or ubiquitin (for PDGFR ubiquitylation assay) antibody (Santa Cruz Biotechnology).

Fyn Kinase Assay.

Fyn kinase activity was quantified using the Universal Tyrosine Kinase Assay Kit (Takara, Madison, Wis., United States). O-2A/OPCs exposed to different treatments were solubilized with an equal volume of the extraction buffer provided with the kit for 15 min, and the resulting lysates were centrifuged at 13,000×g for 15 min at 4° C.; 250 μg of total cell lysates were immunoprecipitated with anti-Fyn antibody (Santa Cruz Biotechnology). Following immunoprecipitation, Fyn immune complexes were washed four times with extraction buffer, and then Fyn kinase activities of each sample were assayed using the kit according to the manufacturer's instructions.

Rho Kinase Assay.

Rho kinase activity was quantified using the CycLex Rho-Kinase Assay kit (MBL international, Woburn, Mass., United States) as described. Cells were lysed and about 500 μg of total cell Jysates were immunoprecipitated with anti-ROCK1 antibody (Sigma), and the precipitates were re-suspended with kinase reaction buffer provided in the kit. Rho kinase activities of each sample were assayed using the kit according to the manufacturer's instructions.

DNA Vector-Based RNA Interference.

siRNA target sites were selected by scanning the cDNA sequence for AA dinucleotides via siRNA target finder (Ambion, Austin, Tex., United States). Those 19-nucleotide segments that start with G immediately downstream of AA were recorded and then analyzed by BLAST search to eliminate any sequences with significant similarity to other genes. The siRNA inserts, containing selected 19-nucleotide coding sequences followed by a 9-nucleotide spacer and an inverted repeat of the coding sequences plus 6 Ts, were made to double-stranded DNAs with Apal and EcoRI sites by primer extension, and then subcloned into plasmid pMSCV/U6 at the Apal/EcoRI site. The corresponding oligonucleotides for the fyn and c-Cbl RNA is are listed in Table 1. Several nonfunctional siRNAs, which contain the scrambled nucleotide substitutions at the 19-nucleolide targeting sequence of the corresponding RNAi sequence, were constructed as negative controls. All of these plasmids were confirmed by complete sequencing.

TABLE 1

Oligonucleotide Sequences for siRNAs

| | |
|---|---|
| Name | Oligo Sequence |

| | |
|---|---|
| Fyn-RNAi-1113- | 5′-GTTTGCTCGACTTCTAAA TTCAAGAGA |
| forward | TTTAAGAAGTCGAGCAAACTT TTTT-3′ |
| | (SEQ ID NO: 1) |
| Fyn-RNAi-1113- | 5′-AATTAAAA |
| reverse | AAGTTTGCTCGAGTTCTTAAA TCTCTTGAA |
| | TTTAAGAAGTCGAGCAAAC GGCC-3′ |
| | (SEQ ID NO: 2) |
| c-Cbl-RNAi-732- | 5′-GTGCATCCCATCAGTTCTG |
| forward | TTCAAGAGA CAGAACTGATGGGATGCACTT |
| | TTTT-3′ |
| | (SEQ ID NO: 3) |
| c-Cbl-RNAi-732- | 5′-AATTAAAA |
| reverse | AAGTGCATCCCATCAGTTCTG TCTCTTGAA |
| | CAGAACTGATGGGATGCAC GGCC-3′ |
| | (SEQ ID NO: 4) |
| Fyn-Scr-RNAi-1113- | 5′-CCCGGGTTTCAATATATTT |
| forward | CTCAAGAGA AAATATATTGAAACCCGGGTT |
| | TTTT-3′ |
| | (SEQ ID NO: 5) |
| Fyn-Scr-RNAi-1113- | 5′-AATTAAAA |
| reverse | AACCCGGGTTTCAATATATTT TCTCTTGAG |
| | AAATATATTGAAACCCGGG GGCC-3′ |
| | (SEQ ID NO: 6) |
| c-Cbl-Scr-RNAi-732- | 5′-TTTAAACCCGGCCCTTTGG |
| forward | TTCAAGAGA CCAAAGGGCCGGGTTTAAATT |
| | TTTT-3′ |
| | (SEQ ID NO: 7) |
| c-Cbl-Scr-RNAi-732- | 5′-AATTAAAA |
| reverse | AATTTAAACCCGGCCCTTTGG TCTCTTGAA |
| | CCAAAGGGCCGGGTTTAAA GGCC-3′ |
| | (SEQ ID NO: 8) |

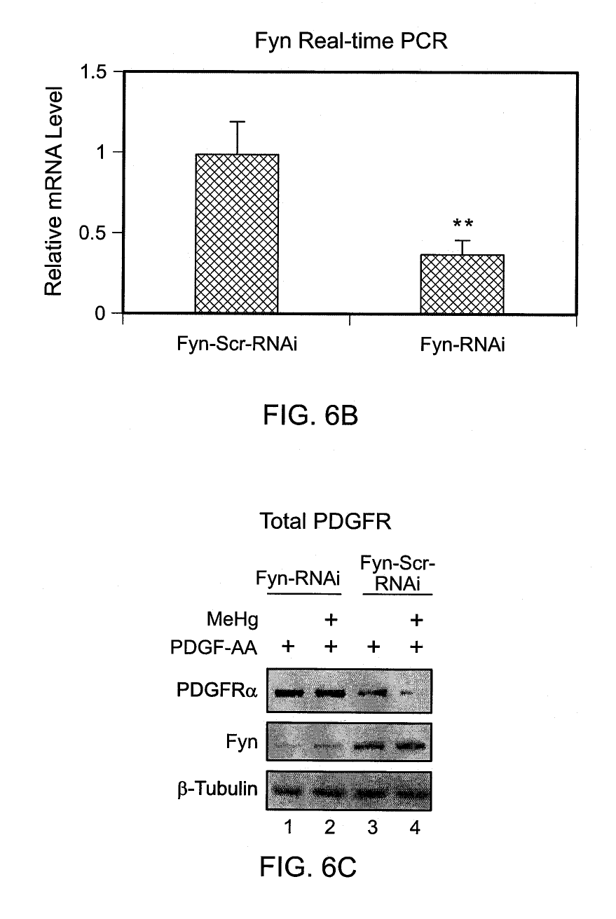

Oligonucleotide sequences for the Fyn and c-Cbl RNAi's used in experments of FIGS. 6 and 8. In addition, nonfunctional siRNAs, which contain the scrambled nucleotide substitutions at the 19-nucleotide targeting sequence of the corresponding RNAi sequence, were constructed as negative controls. All plasmids were confirmed by complete sequencing.

Viral Packaging, Cell Infection, and Selection.

pJEN/neo-HA-70z-c-Cbl plasmids were generously provided by Dr. Wallace Langdon. The pBabe(puro)-HA-70z-c-Cbl plasmids were constructed by transferring the BamH1-digested HA-70z-c-Cbl from pJEN/neo-HA-70z-c-Cbl info the BamH1 digested pBabe(puro) vector. The pBabe(puro)-HA-70z-c-Cbl, pMSCV/U6-Fyn-RNAi, pMCV/U6-c-Cbl-RNAi, and the corresponding scrambled RNAi plasmids and the empty plasmids were transfected into Pheonix Ampho cells by Fugene6 (Roche) transfection solution according to the manufacturer's protocol. Twenty-four hours after transfection, medium was changed to DMEM/F12(SATO, but with no thyroid hormone) supplemented with 10 ng/ml PDGF-AA and bFGF. Virus supernatant was collected 48 h post-transfection, filtered through 0.45-μm filter to remove non-adherent cells and cellular debris, frozen in small aliquots on dry ice, and stored at −80° C. Twenty-four hours prior to infection, O-2AOPCs were seeded. The following day, the culture medium was aspirated and replaced with virus supernatant diluted 1:1 in the O-2A growth media. Medium was then changed into O-2A/OPC growth medium after 8 h or overnight Twenty-four hours after infection, the cells were collected by trypsinization and reseeded in the selective medium (growth medium+200-ng/ml puromycin). By the next day, all non-infected cells were floating and presumably dead or dying. The infected cells were allowed to proliferate for 2 d, and then collected and re-seeded for the following experiments.

RNA Isolation and Real-Time RT-PCR.

Total RNA was isolated using TRIZOL reagent (Inviirogen, Carlsbad, Calif., United States) according to the manufacturer's protocol. A total of 1 μg of RNA was subjected to reverse transcription using Superscript II (In vitrogen). The reactions were incubated at 42° C. for 50 min. The FAM-iabeled probe mixes for rat PDGFRα and Fyn, and the VIC-labeled GAPDH probe mix were purchased from Applied Biosystems (Foster City Calif., United States). For multiplex real-time PCR, reactions each containing 5 μl of 10-fold diluted reverse transcription product, 1 μl of interest gene probe mix. 1 μl of GAPDH probe mix, and 10 μl of TaqMan Universal PCR Master Mix were performed on an iCycler iQ multicolor real-time PCR system (Bio-Rad, Hercules, Calif., United States) and cycling condition was 50° C. for 2 min and 95° C. for 10 min, followed by 40 cycles of 95° C. for 15 sec and 60° C. for 1 min. Each sample was run in triplicate. Data were analyzed, by iCycler iQ software (Bio-Rad).

Clonal Analysis.

O-2A/OPCs purified from P7 rat optic nerve were plated in. poly-L-lysine-coated 25-cm² flasks at clonal density with DMBM medium in the presence of 10-ng/ml PDGF as previously described [9,14,199]. After 24-h recovery, cells were treated with different toxicants, each for 3 d, until visual inspection and immunostaining was performed. NAC was added 1 h before exposure to other toxicants for NAC pretreatment, and NAC co-exists throughout the culture period. The numbers of O-2A/OPCs and oligodendrocytes in each clone were determined by counting under fluorescent microscope. The three-dimensional graph shows the number of clones containing O-2A/OPC cells and oligodendrocytes. Experiments were performed in triplicate in at least two independent experiments.

Animal Treatment.

Six-week-old female SJL mice were treated with MeHg in their drinking water at a concentration of 100 or 250 ppb for 30-60 d prior to mating, and then throughout pregnancy and gestation. This is a level of treatment that is 75%-90% below levels generally considered to be low to moderate and is below levels that have been associated with gross defects in adult or developing animals (e.g., [69-73]).

The exposure levels used in our studies were first determined as candidate exposures from the results of two different previous studies on the relationship between MeHg exposure and levels of toxicant in the brain. Studies by Weiss and colleagues [69] demonstrated that mice exposed to MeHg in their drinking water for up to 14 mo have brain mercury levels roughly equivalent to that in the water. In these studies, mice exposed to MeHg in their drinking water from conception at a concentration of 1 part per million (ppm) had brain levels of MeHg of 1.20 mg/kg (i.e., ppm.) at 14 mo of age, whereas those exposed to MeHg at a concentration of 3 ppm had brain levels of 3.66 mg/kg at this age. It has also been shown, however, that mercury levels in the brain of pre-weanling animals exposed to MeHg via the mother's drinking water throughout gestation and suckling drop rapidly to one-fifth of the levels found at birth, presumably due to reduced MeHg transfer in milk [200]. As an estimated 300,000 to 600,000 infants in the US have blood cord mercury levels of 5.8 μg/l or more [46], and because the human brain concentrates MeHg 5- to 6.7-fold over the concentration occurring in the bloodstream, our goal was to achieve postnatal brain mercury levels of 30 ppb (i.e., ng/g) or less.

In the present study, exposure of female mice to MeHg in their drinking water at a concentration of 250 ppb prior to conception, and maintenance of this exposure during suckling, was associated with brain mercury levels in the offspring (examined at P.14) of 50 ng/g, a fall that was precisely in agreement with predictions based on prior studies on the fall of mercury levels occurring during this period in suckling mice [200]. In offspring of dams exposed to MeHg at a concentration of 100 ppb in the drinking water, brain mercury was below the levels of detection of the Mercury Analytical Laboratory of the University of Rochester Medical Center. The exposure levels of 100 and 250 ppb are 75%-90% below what has otherwise been considered to be low-dose exposure in mice.

Tissue Preparation.

At the time of sacrifice, mice were anesthetized using Avertin (tribromoethanol, 250 mg/kg, 1.2% solution; Sigma) and were perfused transcardiaily with 4% paraformaldehyde in phosphate buffer (pH 7.4) following the removal of the blood by saline solution washing. The brains were removed and stored in 4% paraformaldehyde for 1 d, and then changed to 25% sucrose in 0.1 M phosphate buffer. Brains were cut coronally as 40-μm sections with a sliding microtome (SM/2000R; Leica, Heidelberg, Germany) and stored at −20° C. in cryoprotectant solution (glycerol, ethylene glycol, and 0.1 M phosphate buffer[pH 7.4], 3:3:4 by volume). All animal experiments were conducted in accordance with National Institutes of Health guidelines for the humane use of animals.

In vivo BrdU Incorporation Assay, BrdU Labeling and Olig2 Co-Labeling for BrdU Detection.

To analyze DNA synthesis in vivo, mice were injected with a single dose of 5-BrdU (50 mg/kg body weight), dissolved in 0.9% NaCl, filtered (0.2 μm), and applied intraperitoneally 2 h prior to perfusion. After removal and sectioning of brains, 40-μm free-floating sections were incubated for 2 h in 50% forraamide/2×SSC (0.3 M NaCl and 0.03 M sodium citrate) at 65° C., rinsed twice for 5 min each in 2×SSC, incubated for 30 min in 2N HCl at 37° C., and rinsed for 10 min in 0.1 M boric acid (pH 8.5) at room temperature. Several rinses in TBS were followed by incubation in TBS/0.1% Triton X-100/3% donkey serum (TBS-plus) for 30 min. Sections were then incubated with monoclonal rat anti-BrdU antibody (1:2,500; Harlan Sera-Lab, Loughborough, United Kingdom) and polyclonal rabbit anti-Olig2 in TBS-plus for 48 h at 4° C. Sections were rinsed several times in TBS-plus and incubated for 1 h with donkey anti-rat FITC and donkey anti-rabbit TRITC (Jackson ImmunoResearch Laboratories, West Grove, Pa., United States). After several washes in TBS, sections were mounted on gelatin-coated glass slides using fluoromount-G mounting solution (Southern Biotech).

Quantification of BrdU+ cells was accomplished with unbiased counting methods by confocal microscopy. BrdU immunoreaetive nuclei were counted in one focal plane to avoid over-sampling. In corpus callosum, BrdU+ cells were counted in every sixth section (40 μm) from a coronal, series between interaural AP+5.2 mm and AP+3.0 mm in the entire extension of the rostral and medial part of the corpus callosum. Quantitative data are presented as mean percentage normalized to control animals. Error bars represent±the standard error of the mean (S.E.M).

Images, Data Processing, and Statistics.

Digital images were captured using a confocal laser scanning microscope (Leica TCS SP2). Photomicrographs were processed on a Macintosh G4 and assembled with Adobe Photoshop 7.0 (Adobe Systems, Mountain View, Calif., United. States). Unpaired, two-tailed Student t-test was used for statistical analysis.

Results

Exposure to Environmentally Relevant Levels of MeHg Causes Glial Progenitor Cells to Become More Oxidized and Suppresses Their Division.

The progenitor cells that give rise to the myelin-fonning oligodendrocytes of the central nervous system offer multiple unique advantages for the study of toxicant action, particularly in the context of analysis of toxicant effects mediated by changes in intracellular redox state. These progenitors (which are referred to as both oligodendrocyte-type-2 astrocyte (O-2A) progenitor cells and oligodendrocyte precursor cells, here abbreviated as O-2A/OPCs) are one of the most extensively studied of progenitor cell populations. They also are among a small number of primary cell types that can be analyzed as purified populations, and at the clonal level, and for which there is both extensive information on the regulation of their development and also evidence of their importance as targets of multiple toxicants (including such chemically diverse substances as Pb [2,3], ethanol (e.g., [4-7]), and triethyltin [1,8]).

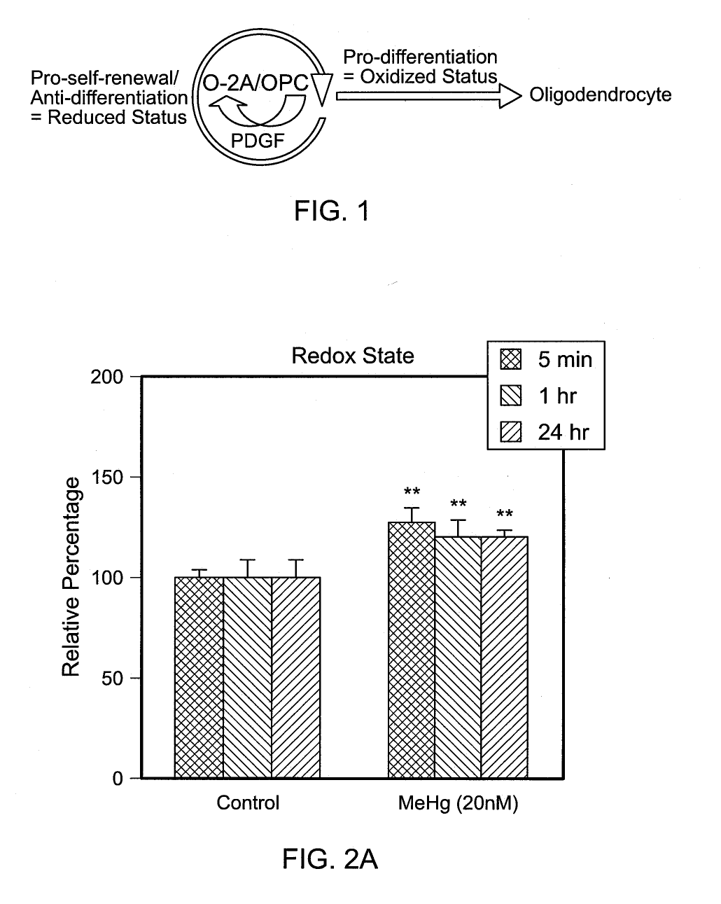

Another important feature of O-2A/0PCs, in regards to the present studies, is that their responsiveness to small (˜15%-20%) changes in intracellular redox state provides a central integrating mechanism for foe control of their division and differentiation [9]. O-2A/OPC-S purified from developing animals on the basis of the cell's intracellular redox state exhibit strikingly different propensities to divide or differentiate. Cells that are more reduced at the time of their isolation undergo extended division when grown in the presence of platelet-derived growth factor (PDGF, the major mitogen for O-2A/OPCs [10-12]), whereas those that are more oxidized are more prone to undergo differentiation [9], Pharmacological agents that make cells slightly more reduced enhance self-renewal of dividing progenitors whereas pharmacological agents mat make cells more oxidized, by as little as 15%-20%, suppress division and induce oligodendrocyte generation. Moreover, cell-extrinsic signaling molecules (e.g., neurotrophin-3 (NT-3) and fibroblast growth factor-2 [FGF-2]) that enhance the self-renewal of progenitors dividing in response to PDGF cause cells to become more reduced. In contrast, signaling molecules that induce differentiation to oligodendrocytes (i.e., thyroid hormone [TH] [13,14]) or astrocytes (i.e., bone morphogenetic protein-4 (BMP-4) [15,16]) cause cells to become more oxidized [9]. The ability of these signaling molecules to alter redox state are essential to their mechanisms of action, as pharmacological inhibition of the redox changes they induce blocks their effects on either division or differentiation of O-2A/OPCs. Thus, multiple lines of evidence have demonstrated that responsiveness to small changes in redox status represents a central physiological control point in these progenitor cells (as summarized in FIG. 1).

MeHg has been previously studied for its effects on neuronal migration, differentiation and survival and on astrocyte function (e.g., [17-24]). Little is known about the effects of MeHg on the oligodendrocyte lineage, despite the fact that there are several reports over the past two decades documenting decreases in conduction velocity in the auditory brainstem response (ABR) of MeHg-exposed children and rats. Such a physiological alteration has long been considered to be indicative of myelination abnormalities in children whose development has been compromised by iron deficiency.

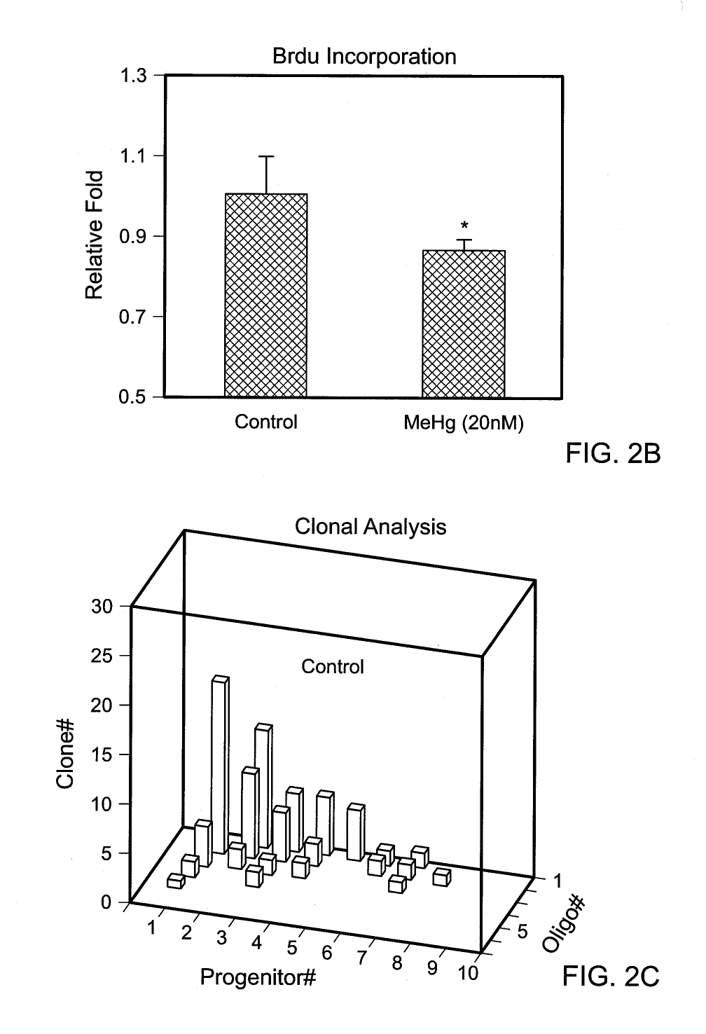

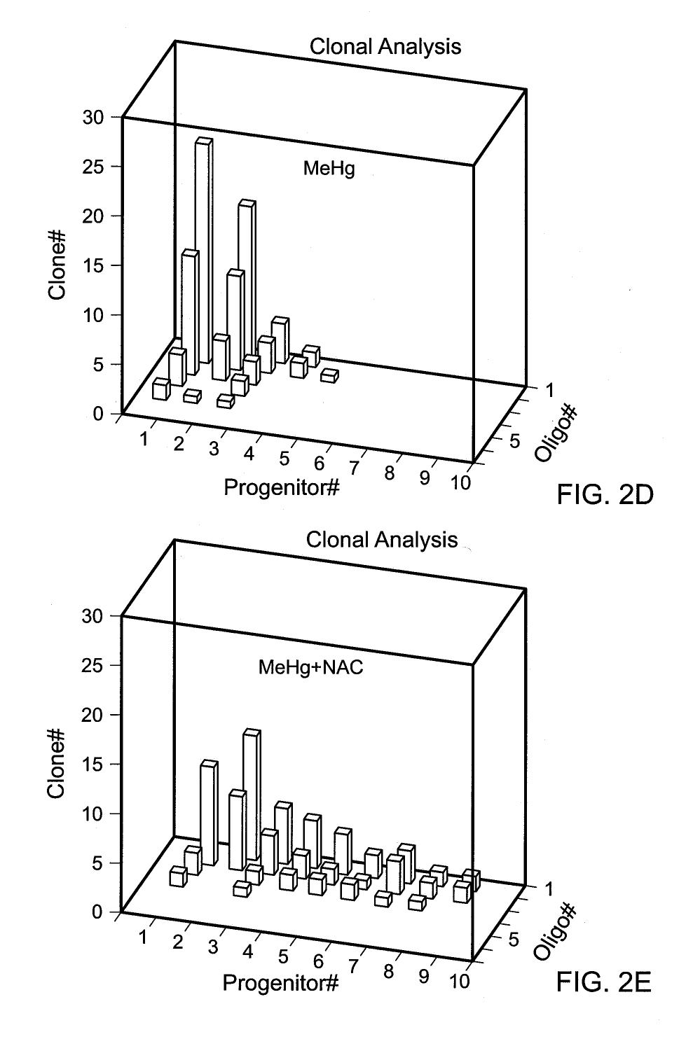

Exposure of O-2A/0PCs (growing in chemically defined medium supplemented with PDGF) to environmentally relevant levels of MeHg makes these cells approximately 20% more oxidized (FIG. 2A), a degree of change similar to that previously associated with reductions in progenitor cell division [9]. Exposure to MeHg inhibited progenitor cell division as determined both by analysis of brornodeoxyuridine (BrdU) incorporation (FIG. 2B) and by analysis of cell division in individual clones of O-2A/OPCs (FIG. 2C-2E). These oxidizing effects of MeHg were seen at exposure levels as low as 20 nM, less than the 5.8 or more μg/1 (i.e., parts per billion [ppb]) of MeHg found in cord blood specimens of as many as 600,000 infants in the US each year and 0.3% or less of the exposure levels previously found to induce oxidative changes in astrocytes. Exposure to 20 nM MeHg was sufficient to cause an approximately 25% drop in the percentage of O-2A/OPCs incorporating BrdU in response to stimulation with PDGF. When examined at the clonal level, MeHg exposure was associated with a reduction in the number of large clones and an increase in the number of small clones, as seen for other pro-oxidant stimuli [9]. Increasing MeHg exposure levels above 50 nM was associated with significant lethality, but little or no cell death was observed at the lower concentrations used in the present studies. Thus, division of O-2A/OPCs exhibits a striking sensitivity to low concentrations of MeHg. MeHg Exposure Reduces the Effects of PDGF from the Nucleus Back to the Receptor.

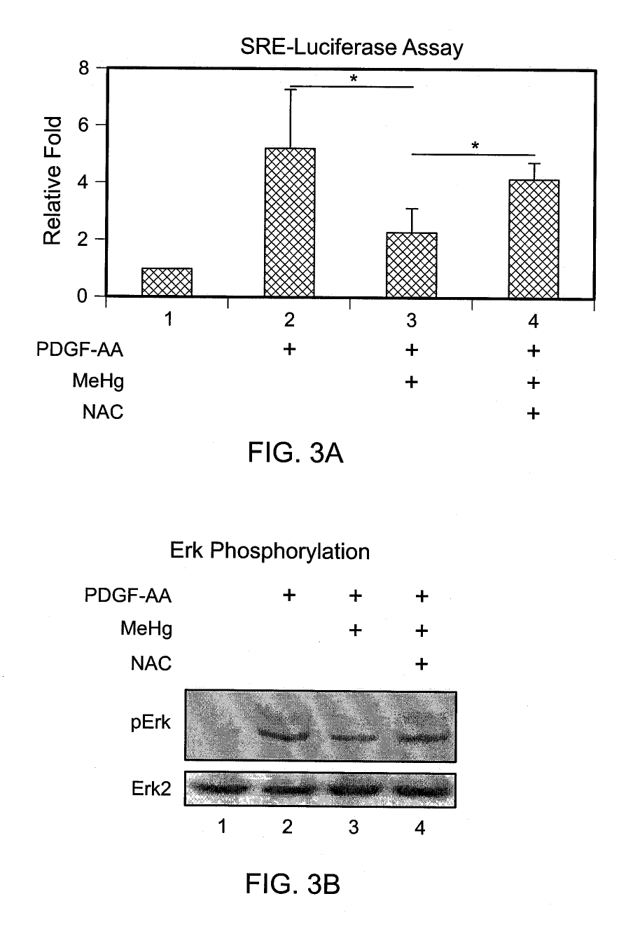

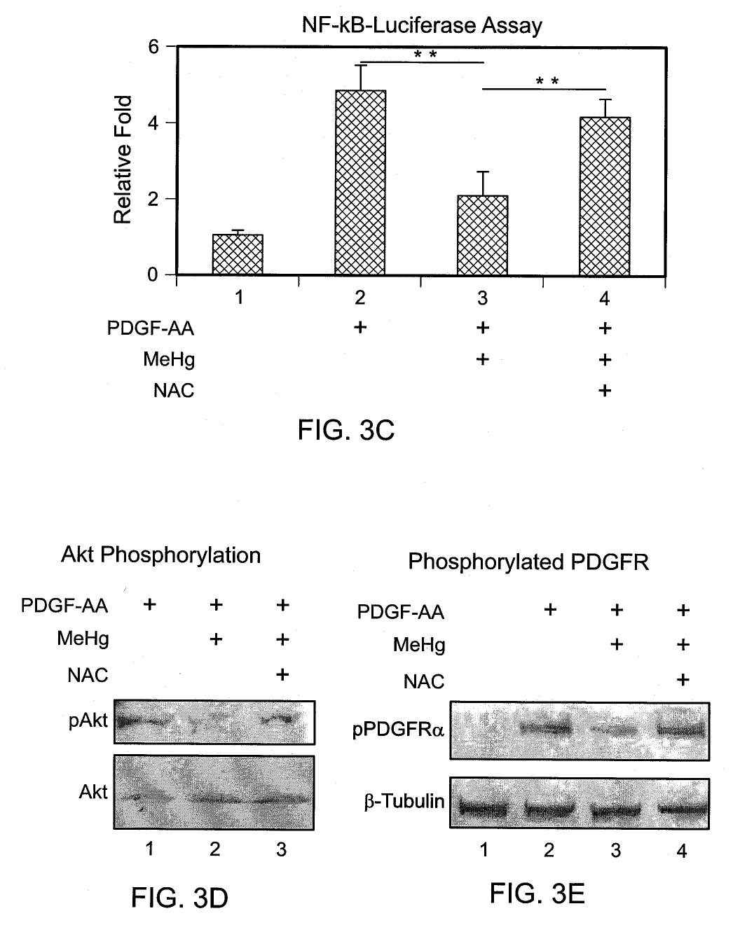

One possible explanation for the reduced division associated with MeHg exposure would be disruption of PDGF-mediated signaling, and molecular analysis revealed that exposure of O-2A/OPCs to 30 nM MeHg for 24 h suppressed PDGF-induced signaling pathway activation, at multiple points from the nucleus back to the receptor. One pathway stimulated by PDGF binding to the PDGF reeeptor-α (PDGFRα ) leads to sequential activation of Raf-1, Raf-kinase, and extracellular signal-regulated kinase 1 and 2 (ERK1/2), which further leads to activation of the Elk-1 transcription factor and up-regulation of immediate early-response gene expression, at least in part through activation of the serum response element (SRE) promoter sequence. MeHg exposure was associated with reduced expression of an SRE-luciferase reporter gene (FIG. 3A), and reduced ERK1/2 phosphorylation (FIG. 3B). PDGFRα activation also stimulates activity of PI-3 kinase, leading to activation of Akt and induction of NF-κB-mediated transcription, both of which also were inhibited by MeHg exposure. Expression of an NF-κB-lueiferase reporter gene was decreased (FIG. 3C), as was phosphorylation of Akt (FIG. 3D). Phosphorylation of PDGFRα, indicating receptor activation, was also reduced in cells exposed to MeHg (FIG. 3E). Because O-2A/OPs growing in these cultures are absolutely dependent upon PDGF for continued division (e.g., [10,11,25]), the suppression of PDGF signaling would necessarily cause a reduction in cell division.

Pathway-Specific Disruption of PDGF-Mediated Signaling, and Reductions in Levels of PDGFRα, Induced by MeHg.

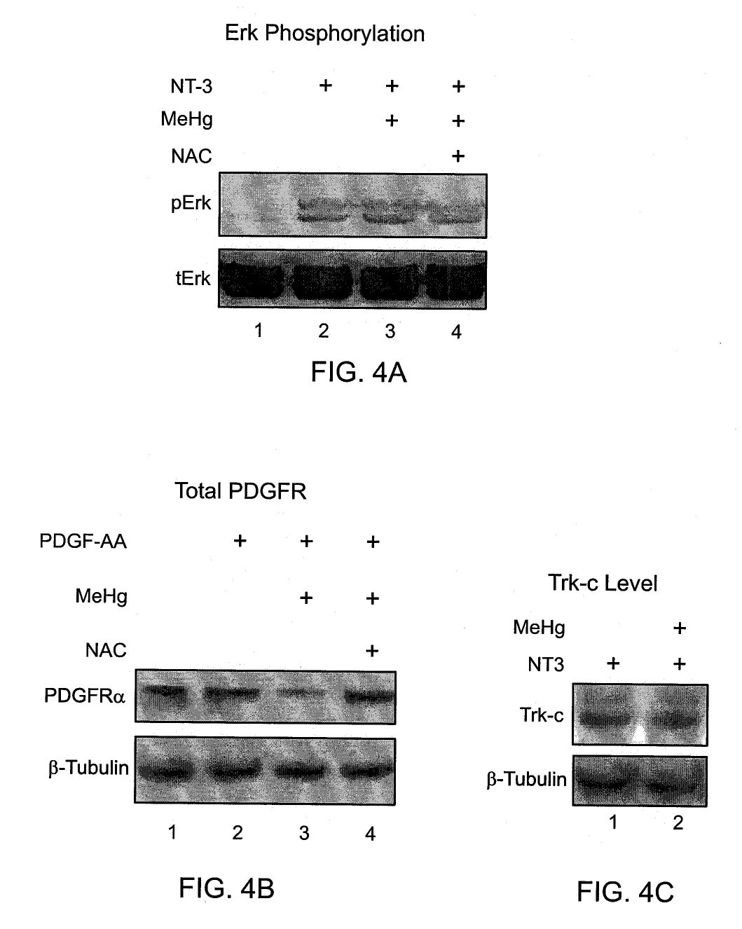

The effects of MeHg were pathway specific and were associated with, reductions in total levels of PDGFRα. O-2A/0PCs exposed to 30 nM MeHg exhibited no reduction in Erk1/2 phosphorylation, induced by exposure to NT-3 (FIG. 4A), and no reduction in NT-3-induced expression from an SRE-luciferase reporter construct. This result shows the site of action of MeHg was upstream of Erk1/2 regulation. Accordingly the effect on PDGFRα was assessed. The reduction in phosphorylated PDGFRα (FIG. 3E) was paralleled by a reduction in levels of the PDGFRα itself (FIG. 4B). In contrast, no reduction in levels of TrkC (the receptor for NT-3 [87]) were caused by exposure to MeHg (FIG. 4C).

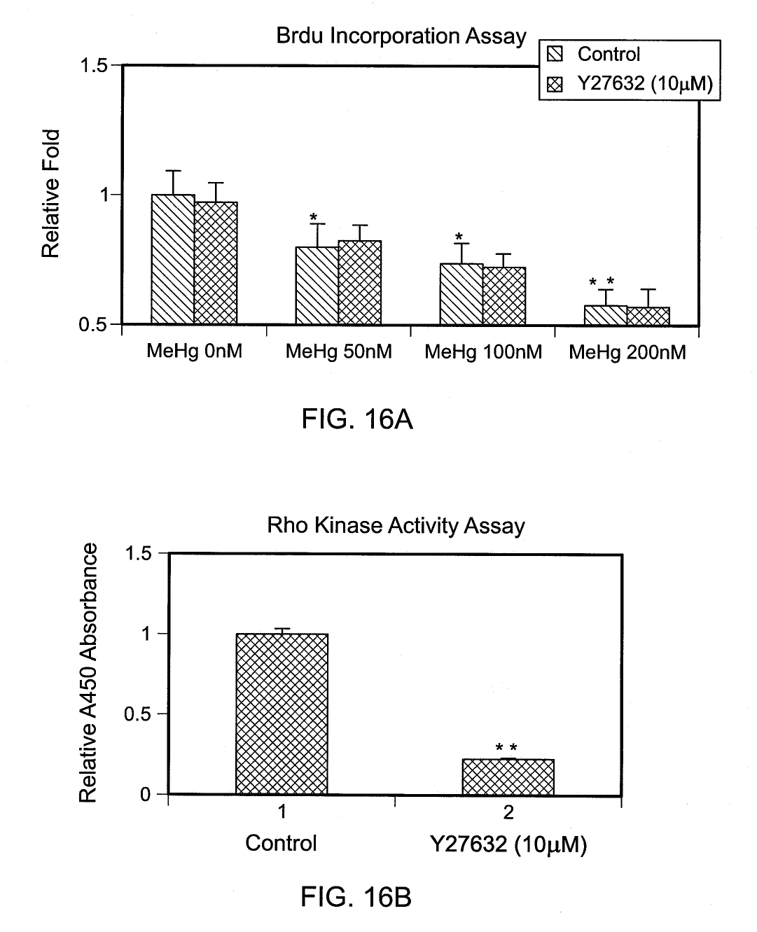

Because Fyn activation in O-2A/OPCs leads to activation of Rho-GTPase, leading to inhibition of Rho kinase activity [41,75], the effects of treatment of cells with the Rho kinase inhibitor Y23762 (FIG. 16) was assessed. Although this agent inhibited Rho kinase activity in O-2A/OPCs, it neither protected against nor exacerbated the effects of MeHg on progenitor cell division (as determined by BrdU incorporation).

Fyn and c-Cbl Activation, and Enhanced Degradation of PDGFRα Induced by MeHg.

One possible explanation for the ability of MeHg to cause a reduction in PDGF-mediated signaling and in total levels of PDGFRα, without affecting NT-3-mediated signaling or TrkC levels, would be that exposure to this toxicant leads to activation of c-Cbl, an E3 ubiquitin ligase that ubiquitylates the activated PDGFRα [27,28], thus leading to its internalization and potential lysosomal degradation [29-31], O-2A/OPCs are known to express Fyn, which has been studied in these cells for its effects on regulation of Rho A activity and control of cytoskeletal organization [40,41]. Because TrkC does not appear to be regulated by c-Cbl, redox-modulated activation of Fyn, leading to c-Cbl activation and enhanced PDGFRα degradation, provided a potential mechanistic explanation integrating the observations reported thus far.

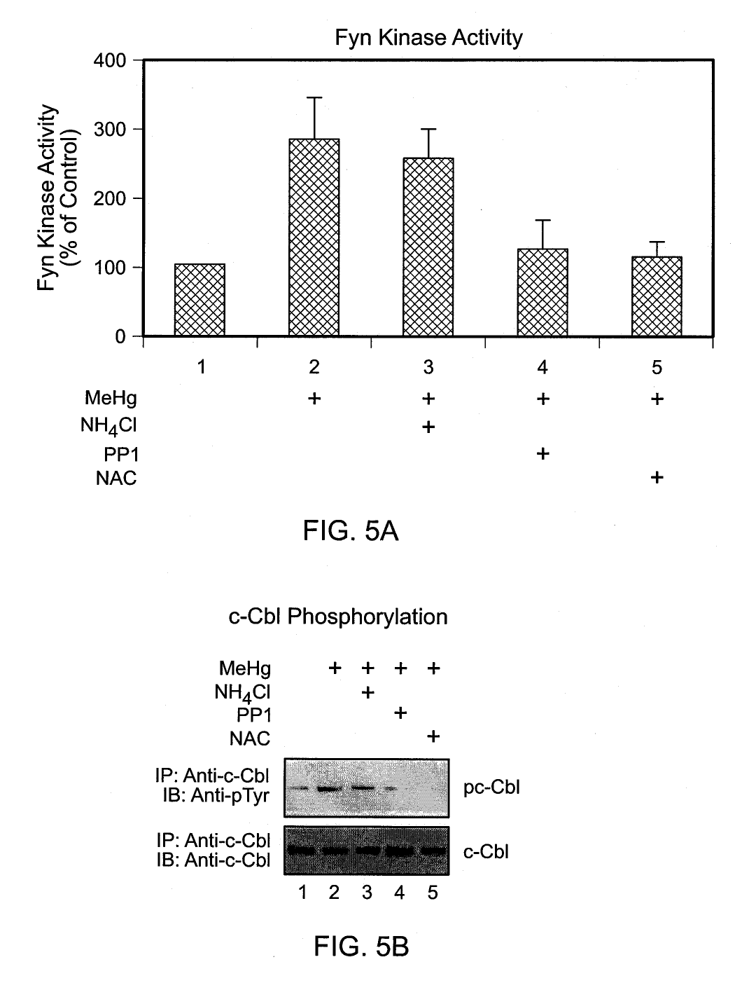

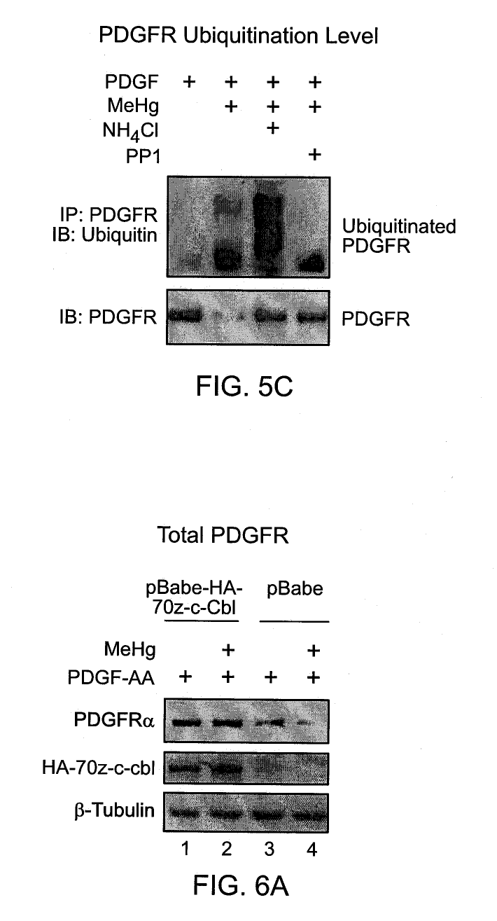

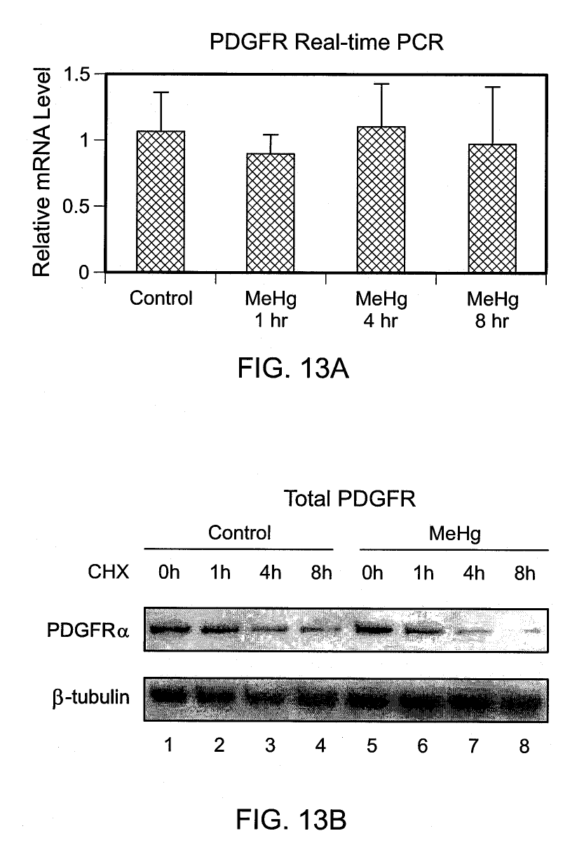

A variety of data show that MeHg exposure activates Fyn, leading to activation of c-Cbl followed by degradation-mediated reductions in levels of activated PDGFRα Exposure of O-2A/OPCs to 30 nM MeHg stimulated Fyn activation and c-Cbl phosphorylation (FIG. 5A and 5B), Activation of Fyn and c-Cbl was blocked by the Src family kinase inhibitors PP1 (FIG. 5A and 5B) and PP2. Exposure to MeHg enhanced ubiquitylation of PDGFRα an increase readily observed even in the presence of markedly reduced levels of the receptor itself (FIG. 5C). Co-exposure to ammonium chloride (NH₄Cl, a lysosomotropic weak base that increases lysosomal pH and disrupts lysosomal protein degradation) prevented receptor degradation, and was associated with increased levels of ubiquityiated receptor in treated O-2A/OPCs. The increase in levels of ubiquitylated receptor was consistent with the lack of effect of NH₄Cl on either Fyn activation or c-Cbl phosphorylation (FIG. 5A and 5B). Treatment with PP1, which inhibits Fyn activity (FIG. 5A), was also associated with a marked reduction in the amount of ubiquityiated PDGFRα particularly in comparison with levels of total receptor (compare upper and lower lanes in FIG. 5C). As further confirmation that reductions in levels of PDGFRα were due to protein degradation, exposure to MeHg did not have any significant effects on levels of PDGFRα mRNA, as determined by quantitative PGR analysis (FIG. 13A). In the presence of cycloheximide, an inhibitor of protein synthesis, MeHg further accelerated receptor loss as compared with that occurring solely due to failure to synthesize new protein (FIG. 13B). Collectively, these results indicate that MeHg enhances active degradation of PDGFRα, as contrasted with reducing receptor levels as an indirect consequence of altering transcriptional or translational regulation of receptor levels.

Molecular confirmation of the role of Fyn and c-Cbl in the effects of MeHg on levels of PDGFRα was obtained by expression of dominant negative c-Cbl, or small inhibitory RNA (RNAi) for Fyn or Cbl, in MeHg-exposed O-2A/0PCs. Expression of the dominant-negative (DN) 70z mutant of c-Cbl [42-44] in O-2A/OPCs prevented MeHg-induced reductions in levels of PDGFRα (FIG. 6A). Reduction in levels of Fyn protein by introduction of Fyn-specific small interfering RNA (siRNA ) constructs ((FIG. 6B) also protected against MeHg-induced reductions in levels of PDGFRα (FIG. 6C), as predicted by the hypothesis that MeHg-induced activation of Fyn mechanistically precedes reductions in receptor levels. Similar results were obtained using RNAi constructs for c-Cbl, as shown below.

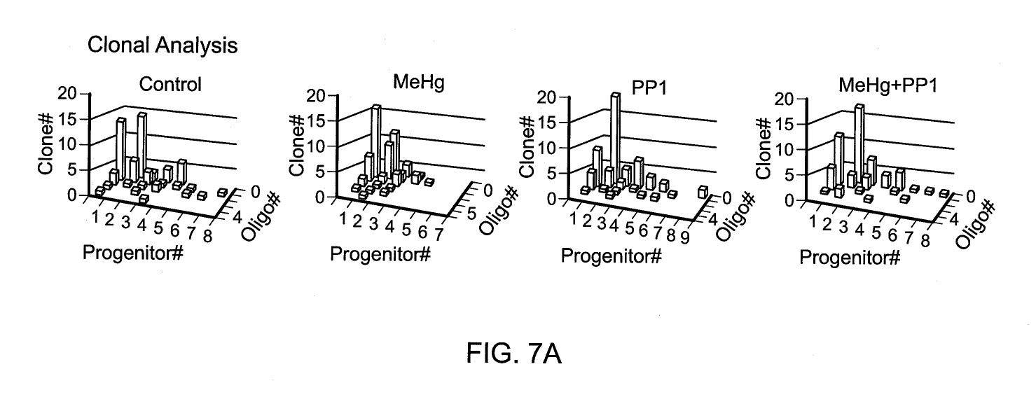

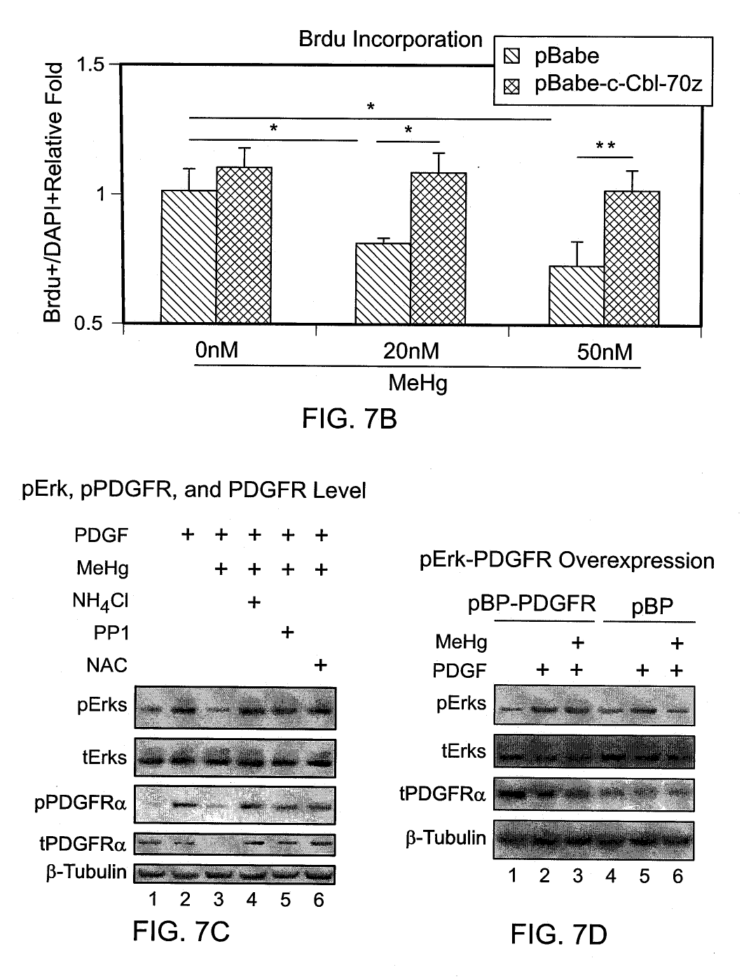

Suppression of Fyn or c-Cbl activity, or overexpression of PDGFRα itself also protected against the functional effects of MeHg exposure (FIG. 7). Pharmacological inhibition of Fyn activity with PP1 enabled analysis of O-2A/OPC division at the clonal level, and demonstrated that PP1 blocked MeHg-induced suppression of cell division (FIG. 7A). O-2A/OPCs expressing DN-70Z-c-Cbl and exposed to MeHg were also protected from effects of MeHg on cell division, as analyzed, by BrdU incorporation (FIG. 7B), Co-treatment of MeHg-exposed O-2A/OPCs with PP1 or NH₄Cl also blocked MeHg-associated suppression of Erk1/2 phosphorylation (and MeHg-induced reductions in levels of PDGFRα, indicating that Erk1/2 suppression was a secondary consequence of the effects of Fyn and c-Cbl activation (FIG. 7C). Overexpression of PDGFRα in MeHg-exposed O-2A/OPCs also protected cells from MeHg-associated reductions in Erk1/2 phosphorylation (FIG. 7D).

Convergence of Chemically Diverse Toxicants on Activation of Fyn and c-Cbl and Reductions in Levels of PDGFRα.

To determine whether effects of MeHg revealed a general mechanism by which chemically diverse toxicants with pro-oxidant activity could alter cellular function in similar ways, the effects of exposure of dividing O-2A/OPCs to Pb (a heavy metal toxicant) and paraquat (an organic herbicide) were examined. These toxicants both make cells more oxidized, but through mechanisms that differ between them and also from effects of MeHg.

Despite their chemical differences from MeHg, and from each other, Pb and paraquat had apparently identical effects as MeHg on ERK1/2 phosphorylation, activation of Fyn and c-Cbl, and reductions in levels of phosphorylated PDGFRα and on total levels of PDGFRα (FIG. 8), O-2A/OFCs were exposed to 1 μM Pb (equivalent to the levels of 20 μg/dl known to be associated with cognitive impairment, and a level of Pb previously found to inhibit O2A/0PC division without causing cell death [2,3,45]) or to 5 μM paraquat (an exposure level selected as being in the lowest 0.1% of the range of paraquat concentrations studied by others in vitro, which range from 8 μM-300 mM (e.g., [46, 47]), Pb and paraquat, exposure at these levels did not cause cell death, but did make O-2A/OPCs approximately 20% more oxidized, as determined by analysis of cells with the redox-indicator dyes dihydro-chloromethyl-rosamine or dihydro-caicein-AM. Both Pb and paraquat exposure were associated with activation of Fyn (FIG. 8 A), increased phosphorylation of c-Cbl. (FIG. 8B), reduced levels of Erk1/2 phosphorylation, and reduced, levels of phosphorylated and total PDGFRα (FIG. 8C). As for MeHg, the effects of Pb and paraquat on PDGFRα levels were prevented by expression of RNAi for c-Cbl (FIG. 8D), DN(70Z) c-Cbl, or RNAi for Fyn.

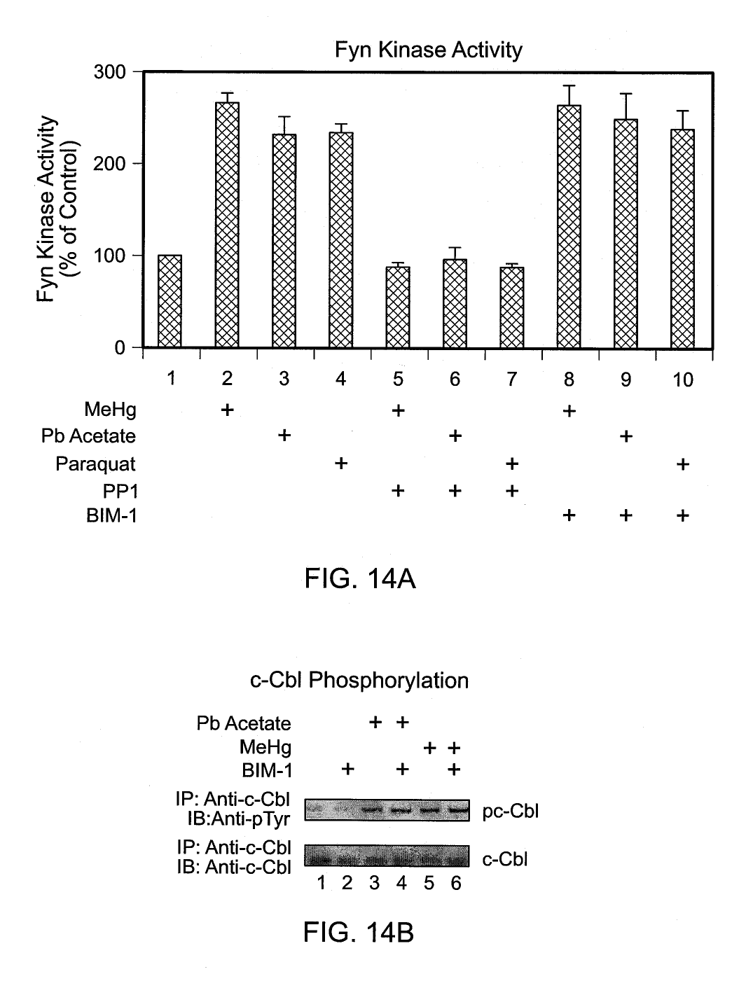

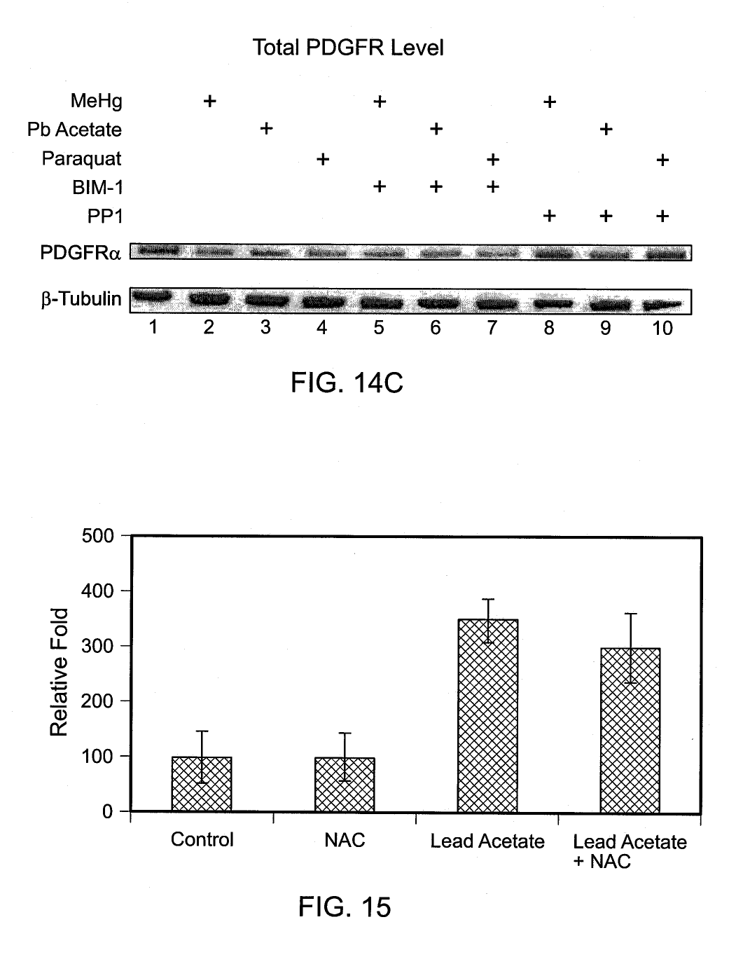

It has previously been suggested that the effects of Pb on O-2A/OPCs are mediated through activation of PKC [2], a pathway that has not been, implicated in the activity of MeHg or paraquat. To determine whether PKC inhibition could distinguish between effects of Pb versus MeHg or paraquat, and to determine if PKC activation was relevant to the effects of toxicants on Fyn or c-Cbl activation or reductions in PDGFRα levels, the effects of co-exposure of O-2A/OPCs to bisindoleylmalehnide I (BIM-1, a broad-spectrum PKC inhibitor previously used in the analysis of the role of PKC activation in the effects of Pb on O-2A/OPCs [2]) were examined. As shown, in FIG. 14, co-exposure of O-2A/OPCs to BIM-1 with Pb, MeHg, or paraquat did not prevent toxicant-mediated activation of Fyn (FIG. 14A) or c-Cbl (FIG. 14A). BIM-1 co-exposure also did not protect against MeHg-, Pb- or paraquat-induced reductions in levels of PDGFRα (FIG. 14C).

Protection by Cysteine Pro-Drugs.

If it is correct that Fyn activation, with its consequences, is regulated by the ability of toxicants to make cells more oxidized, then antagonizing such redox changes should prevent Fyn activation. Previous studies have shown that an effective means of preventing the increase in oxidative status and the suppression of cell division caused by exposure of O-2A/OPCs to TH is to treat cells with N-acetyl-L-cysteine (NAC), a cysteine pro-drug that is readily taken up by cells and converted to cysteine [9]. Cysteine is the rate-limiting precursor for synthesis of glutathione, one of the major regulators of intracellular redox status. NAC also possesses anti-oxidant activity, has long been used as a protector against many types of oxidative stress, and has been shown to confer protection against a wide range of toxicants, including MeHg (e.g., [48-50]), Pb (e.g., [9,51,52]), and paraquat (e.g., [53]), as well as such other substances as aluminum [54], cadmium [55], arsenic [56], and cocaine [57].

The pro-oxidant activities of chemically diverse toxicants are causal in Fyn activation, and NAC was equally effective at preventing Fyn activation and its consequences induced by exposure to MeHg, Pb, or paraquat (FIGS. 2-5, 7, and 8), For cells grown at the clonal level, NAC blocked the suppressive effects of MeHg on cell division (FIG. 2). NAC also blocked all effects of MeHg on PDGF-mediated signaling, and rescued normal level of activity of SRE and Nf-κB promoter-reporter constructs and levels of phosphorylation of Erk1/2, Akt, and PDGFRα (FIG. 3). NAC also blocked MeHg-induced activation of Fyn and phosphorylation of c-Cbl (FIG. 5), and prevented MeHg-induced reductions in levels of PDGFRα (FIG. 4). Critically, for the hypothesis that Pb and paraquat effects also were mediated by changes in redox state, NAC also blocked the effects of Pb and paraquat, on Fyn activation and c-Cbl phosphorylation, and protected against effects of these toxicants on Erk1/2 phosphorylation and levels of PDGFRα (FIG. 8). Levels of PDGFRα were also protected by exposure of O-2A/OPCs to proeysteinc (FIG. 8), a thiazolidine-derivative cysteine pro-drug that differs from NAC in having no intrinsic anti-oxidant activity [58]. To determine whether the ability of cysteine pro-drugs to protect against the effects of MeHg, Pb, and paraquat was due to enhanced toxicant clearance associated with elevated levels of glutathione, analysis of Pb uptake, levels of Pb were analyzed in the presence and absence of NAC. Leadmium Green AM (a fluorescent indicator of Pb levels) showed no significant difference in Pb levels between cells exposed to Pb as compared with cells exposed to Pb and NAC (FIG. 15). Such protection can also be achieved with other glutathione prodrugs, such as other cysteine derivatives that are taken up by cells.

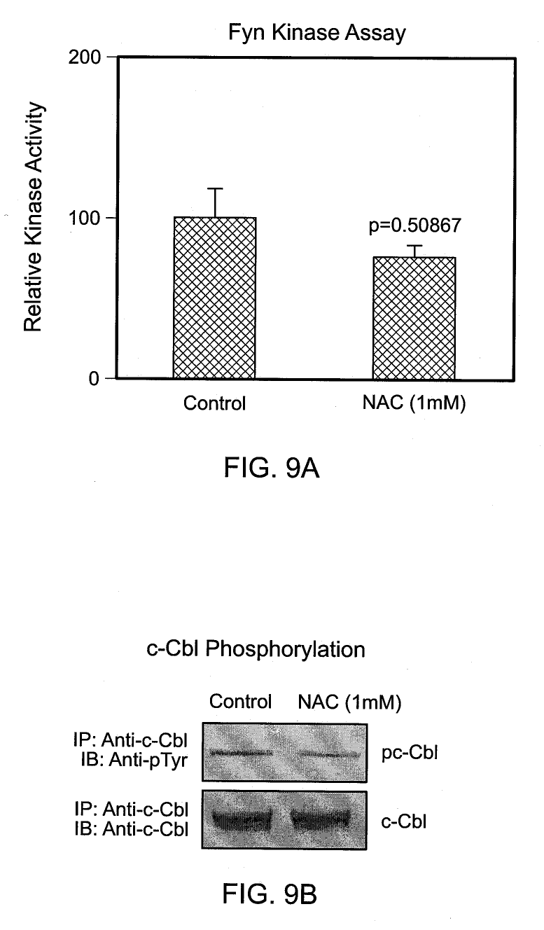

The ability of NAC to block toxicant-induced activation of Fyn raises the question of whether this is due to a true prevention of the effects of toxicant exposure on activation of this kinase or, alternatively, is due to an ability of NAC to independently suppress Fyn activity to such an extent that the apparent block of toxicant effects instead represented the summation of two opposing influences of equivalent magnitude. To evaluate these two possibilities, O-2A/OPCs were exposed to 1 mM NAC in the absence of toxicants, and Fyn and c-Cbl activation were evaluated as in FIG. 5. NAC exposure had only a slight, insignificant effect on the levels of basal Fyn activity in O-2A/OPCs (FIG. 9A). In agreement with this outcome, NAC exposure did not have any marked effect on levels of c-Cbl phosphorylation (FIG. 9B). Thus, it appears that NAC-mediated counteraction of the effects of toxicants on Fyn activation is tar greater in its magnitude than its direct effects on basal levels of Fyn activity.

Toxicants cause reductions in levels of other c-Cbl targets.

The general effect of toxicants on activation of the Fyn/c-Cbl pathway was further tested by determining whether other c-Cbl targets were affected similarly to PDGFRα. One member of the c-Cbl interactome. [31] known to be expressed by O-2A/OPCs is c-Met. [59], the receptor for hepatocyte growth factor (EGF). Oligodendrocytes also have recently been reported to be responsive to epidermal growth factor (EGF) application with morphological changes [60], and microarray analysis confirms that the EGF receptor (EGFR) is expressed by O-2A/OPCs. The EGFR is perhaps the most extensively studied RTK target of c-Cbl [29,35,43,61-63], but c-Met regulation by c-Cbl appears to follow similar principles [42,64].

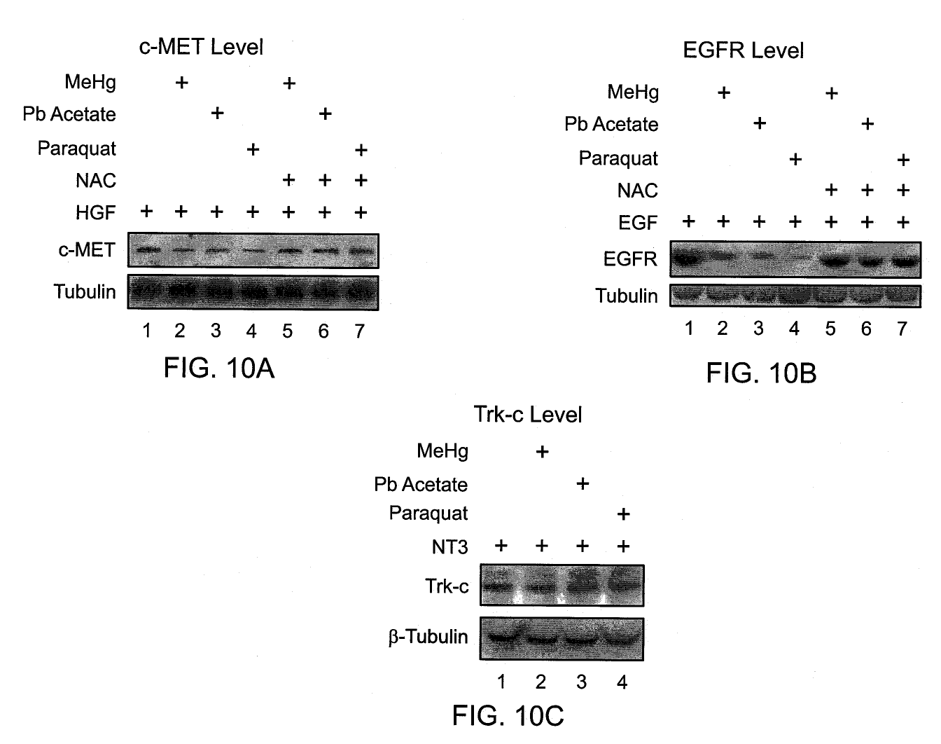

As shown in FIG. 10, exposure of O-2A/OPCs to MeHg was associated with reductions in levels of c-Met (FIG. 10A) and EGFR (FIG. 10B). Consistent with the data showing that Pb and paraquat converge with MeHg on activation of the Fyn/c-Cbl pathway, levels of C-Met and EGFR were also reduced in O-2A/OPCs exposed to these additional toxicants. NAC protected both c-Met and EGFR levels from reductions associated with exposure to MeHg, Pb, or paraquat.

Further support for the Fyn/c-Cbl hypothesis of toxicant convergence was provided by observations that neither Pb or paraquat caused a reduction in levels of TrkC (FIG. 10C), just as observed for MeHg (FIG. 4C).

Developmental Exposure to Low Levels of MeHg In Vivo Causes Reductions in Levels of PDGFRα and EGFR, But Not TrkC, and Causes Reduced Division of O-2A/OPCs

In vivo were performed to determine whether toxicant exposure was associated with specific reductions in RTKs that are c-Cbl targets, whether this occurred at levels of toxicant exposure approximating the effects of environmental exposure, and whether such exposure caused subtle changes in O-2A/OPC function. By way of example, these experiments were conducted with MeHg. To test the hypothesis that environmentally relevant levels of MeHg exposure cat perturb the developing CNS in subtle ways, SJL mice were exposed to 100 or 250 ppb MeHg in their drinking water throughout gestation, and maintained this exposure until sacrifice of pups at 7 and 21 d after birth. As discussed in above, these exposure levels approximated tire predicted mercury levels in the CNS of 300,000-600,000 infants in the US. The exposure levels examined in the present study was 75%-90% below what has otherwise been considered to be low-dose exposure in mice.

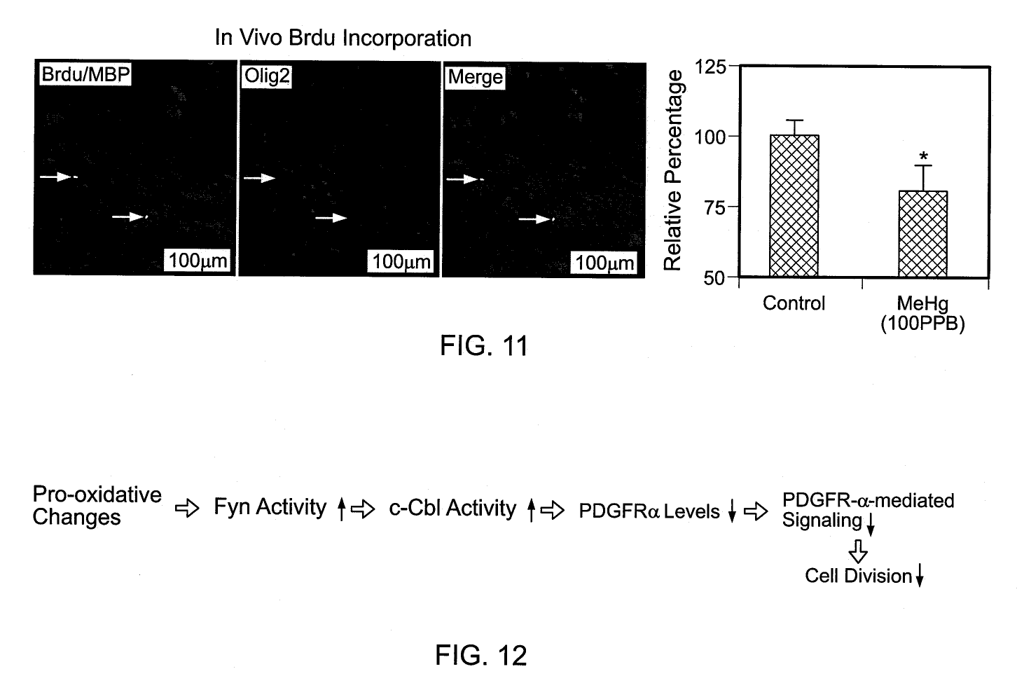

Developmental exposure of mice to MeHg at either 100 ppb or 250 ppb in the maternal drinking water was associated with clear and significant reductions in levels of PDGFRα and EGFR, but not of TrkC (FIG. 11). Treatment of SJL mice with 100 or 250 ppb MeHg in the drinking water during gestation and suckling was associated with reductions in levels of PDGFRα and EGFR in the cerebellum, hippocampus, and corpus callosum when brain tissue was sampled at 7 and 21 d after birth. In contrast, levels of the NT-3 receptor TrkC were not reduced in these animals, as predicted by our in vitro analyses. It was particularly striking that exposure even to 100 ppb MeHg in the drinking water was enough to have significant effects on levels of PDGFRα and EGFR. These changes, and the lack of effect of MeHg exposure on TrkC levels, are consistent with the in vitro analyses.

Analysis of BrdU incorporation revealed that these low levels of MeHg exposure also were associated with, statistically significant reductions in the division of O-2A/OPCs in vivo. In these experiments, postnatal day 14 (P14) animals were treated as for analysis of receptor levels except that BrdU was administered 2 h before sacrifice. Sections then were analyzed with anti-BrdU antibodies to identify cells engaged in DNA synthesis and with antibodies to olig2 to identify O-2A/OPCs (as in [74]). 0lig2 is a transcriptional, regulator expressed in oligodendrocytes and their ancestral precursor cells. In white matter tracts of the CNS, BrdU+ cells that express Olig2 are considered to be O-2A/OPCs. In the present study, greater than 90% of all BrdU+ cells in the corpus callosum were also Olig2+. When the number of Olig2+/BrdU+ cells found in the corpus callosum of control and experimental animals was analyzed (see above for details of analysis), a 20% reduction in the number both of total BrdU+ cells and of Olig2+/BrdU+ cells (FIG. 11) was detected. This outcome is consistent with the results of the in vitro studies (FIG. 2B).

### Example 2

### Chemoprotection by Interrupting the Redox/Fyn/c-Cbl Pathway

Killing of normal cells, but not of cancer cells, by chemotherapeutic agents, utilizes activation of the redox/Fyn/c-Cbl pathway, thus providing a means of conferring selective protection on normal cells. The adverse effects of chemotherapeutic agents on the normal cells of the body have been long recognized. Both precursor cells and the myelin-forming oligodendrocytes of the brain are also more vulnerable to a variety of chemotherapeutic agents than are cancer cells. See, e.g., Dietrich J, Han R, Yang Y, Mayer-Proschel M, Noble M, (2006), CNS Progenitor Cells and Oligodendrocytes are Targets of Chemotherapeutic Agents In Vitro and In Vivo, J Biol. 5(7):22. Regulation of the redox/Fyn/c-Cbl pathway provides a means of achieving selective protection of normal cells without protecting cancer cells. Results are presented herein for tamoxifen (TMX), by way of example only.

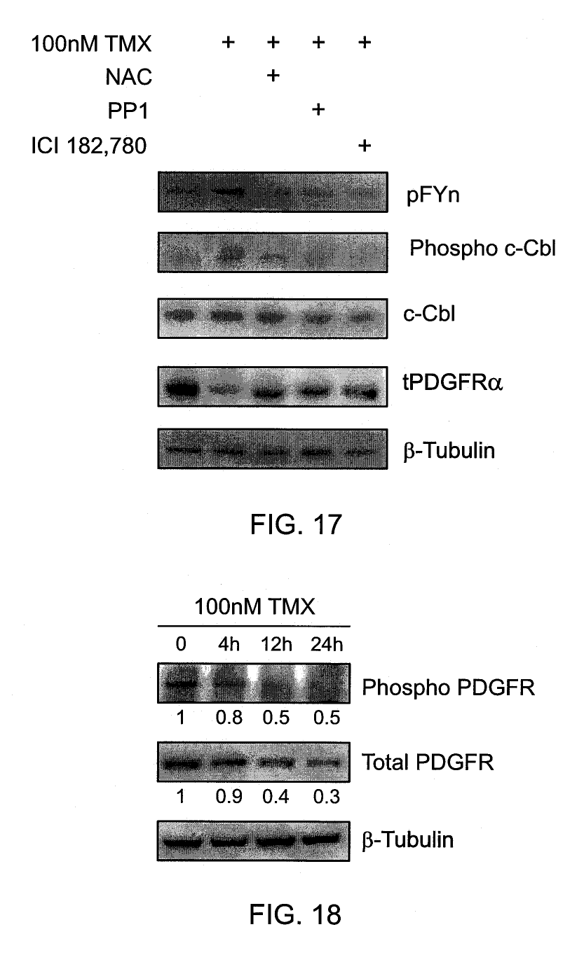

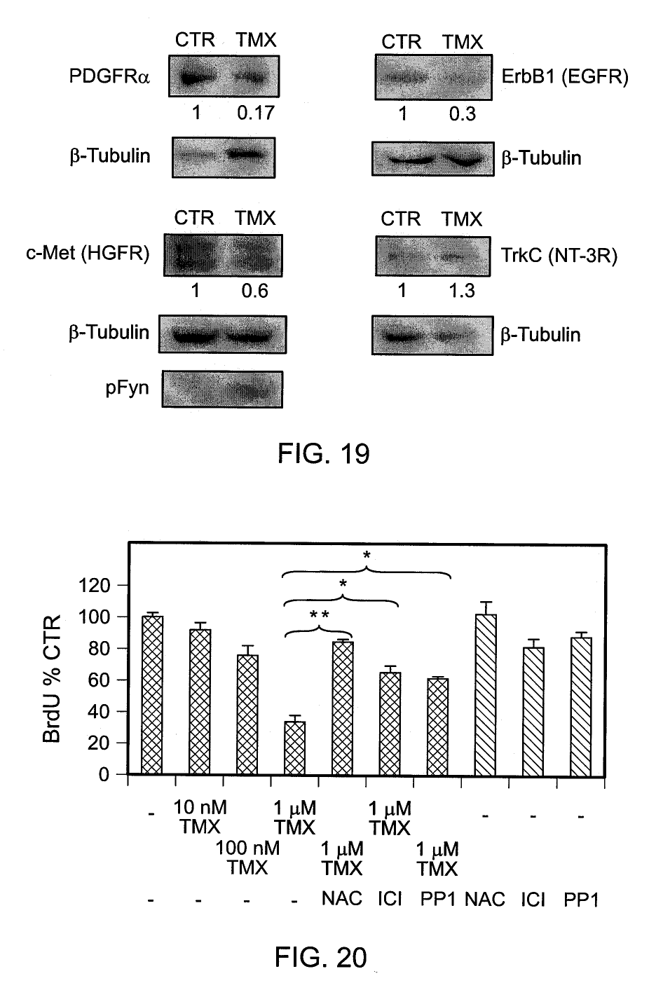

Freshly isolated O-2A/OPCs,as described in Example 1 were used in these experiments. Cultures were treated with TMX (10 nM, 100 nM and 1 μM) for 2 days. In some experiments NAC (1 mM), ICI 182,780 (200 nM), or PP1 (100 nM) were added concurrently to 1 uM TMX cultures. The effect of TMX on activation of Fyn and c-Cbl and on levels of PDGFRα (total and phosphorylated) were measured as described in Example 1 (FIG. 17). β-tubulin levels were measured as an internal control. Levels of total and phosphorylated PDGFRα show a parallel reduction over time with exposure to TMX (4, 12, 24 hours), whereas levels of tubulin are unchanged. (FIG. 18). Like PDGFRα, C-Met and EGFR are decreased following TMX exposure, whereas levels of Trk (a non-c-Cbl target) show no differences with TMX treatment. (FIG. 19). BrdU analysis, as described above, shows that the TMX effect on cell proliferation is blocked by NAC, PP1, ICI 182,780. (FIG. 20).

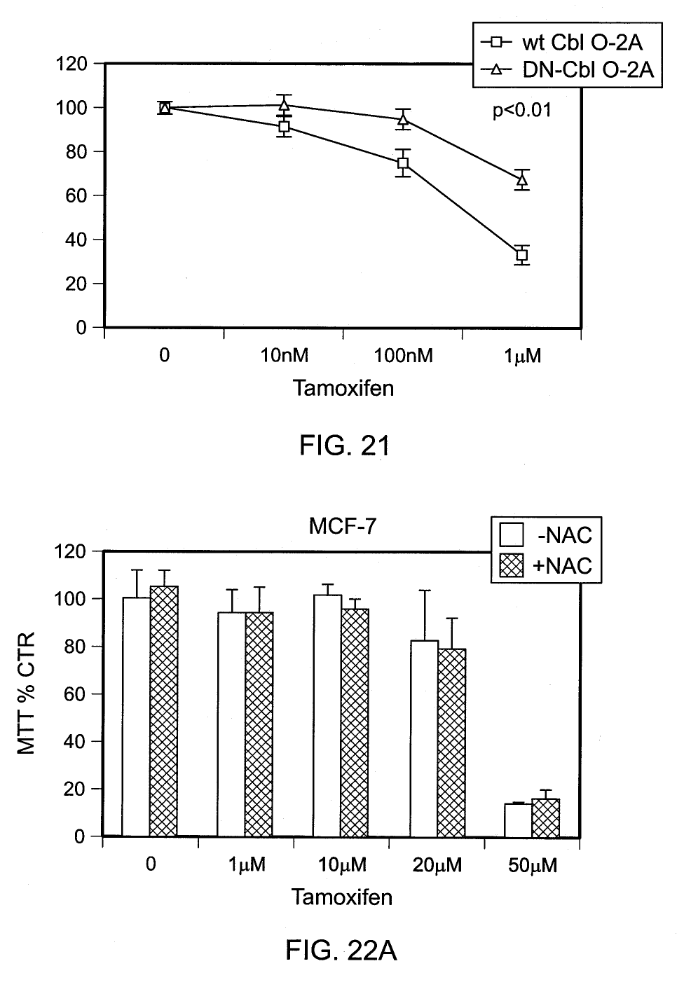

Suppression of O-2A/OPC division by TMX exposure was also attenuated by preventing c-Cbl activity with expression of the dominant negative (70Z) mutant of c-Cbl. TMX's effect on cell division was analyzed in normal O-2A/OPCs and O-2A. OPCs stably expressing dominant-negative c-Cbl (70z-c-Cbl). pBabe-HA-70z-c-Cbl was introduced to freshly isolated O-2A/OPCs via retroviral infection and cells that stably expressed the mutant c-Cbl were generated by drug selection. Both normal and 70z-c-Cbl-containing O-2A/OPCs were exposed to TMX (10 nM, 100 nM and 1 μM) for 2 days, and the percentage of dividing O-2A/OPCs was determined by BrdU incorporation analysis combined with A2B5 staining. (FIG. 21).

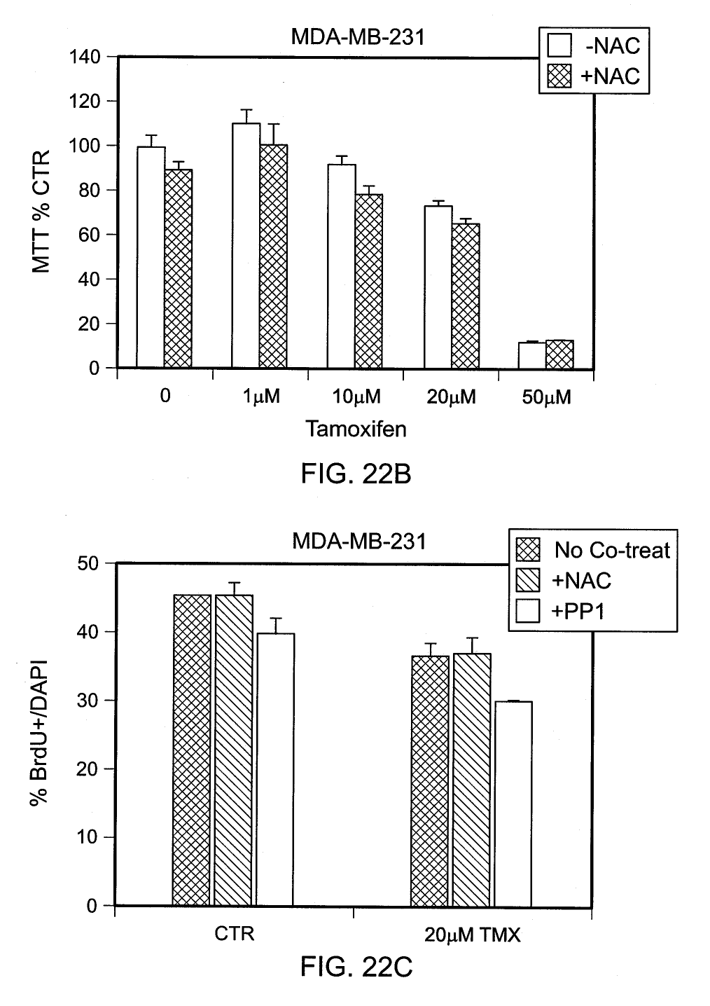

In contrast to O-2A cells, TMX effects on cancer cells are not blocked by NAC or PP1, as shown in FIG. 22. It has been reported that c-Cbl induced down-regulation of EGFR is disabled in cancer cells (Hirsch DS, Shen. Y, Wu WJ, (2006) Cancer Res. Apr 1;66(7):3523-30). Thus, growth and motility inhibition of breast cancer cells by EGFR degradation is correlated with inaetivation of Cdc42. As shown in FIG. 22A and B, cancer cell viability was analyzed using MTT assays. Cells were cultured in phenol red-free growth media and exposed to TMX (0, 1 μM, 10 μM, 20 μM and 50 μM) for 48 hours, with or without 1 mM NAC. In FIG. 22C. MDA-MB-231 cell division was analyzed by BrdU incorporation analysis. Cells were cultured in phenol red-free growth medium and exposed to 20 μM tamoxifen for 48 hours, which caused approximately a 10% reduction in cell division. Co-treatment with NAC (1 mM) or PP1 (200 nM) did not result in any rescue effect. Percentage of Brdu+ cells from DAPI+ cells was determined in each sample.

### Example 3

### Precursor Cell Expansion by Interruption of the Redox/Fyn/c-Cbl Pathway

The redox/Fyn/c-Cbl pathway plays a critical role in modulating the balance between division and differentiation, and inhibition of this pathway provides a means of causing extended precursor cell division while suppressing differentiation. O-2A/OPCs growing in the presence of PDGF (10 ng/ml) were exposed to TH (T3 at 0.45 μM+T4 at 0.49 μM), BMP4 (10 ng/ml), FGF-2 (10 ng/ml) or NT-3 (10 ng/ml) for 24 hr. Cells were starved without PDGF for 8 hr and then treated with 10 ng/ml of PDGF-AA or NT-3 (10 ng/ml) for 1 hr. Cells were iysed for Western Blot analysis. Samples were resolved by SDS-PAGE gels and transferred to PVDF membranes (PerkinElmer Life Science), After being blocked in 5% skim milk in PBS containing 0.1% Tween 20, membranes were incubated with primary antibodies against total or phosphorylated Erks, followed by incubation with HRP-conjugated secondary antibody (Santa Cruz). Membranes were visualized using Western blotting Luminol reagent (Santa Cruz).

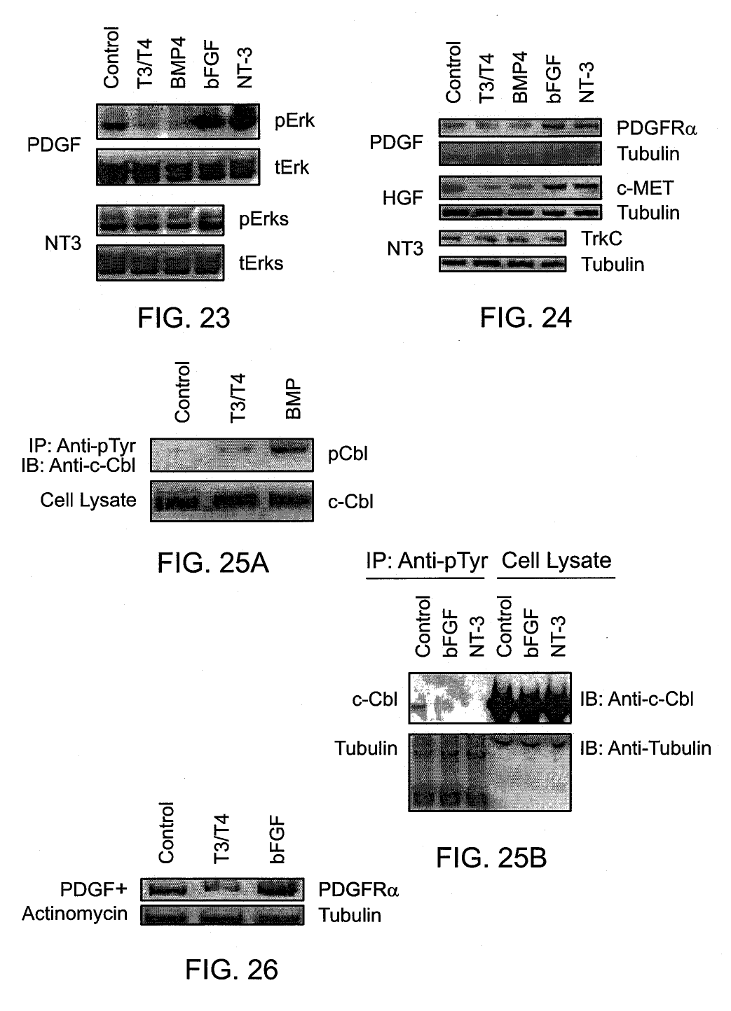

As shown in FIG. 23, cell-extrinsic modifiers of the balance between division and differentiation cause pathway specific alterations in signaling by receptor tyrosine kinases. PDGF-mediated signaling appears to be altered by TH, BMP, NT-3, FGF-2 at multiple points in the signaling pathway, consistent with Example 1 regarding the effects of toxicants on the pathway. Effects of PDGF in the nucleus were examined with reporter constructs for the serum response element (SRE) and for NF-κB. Further upstream analysis examined phosphorylation of Erk1/2. FIG. 23 demonstrates that exposure to TH or BMP-4 greatly reduces SRE-reporter gene expression. Similar results were obtained with Nf-κB reporter constructs. FIG. 23 also demonstrates these agents reduce Erk1/2 phosphorylation caused by PDGF, while co-exposure to NT-3 or FGF-2 enhances signaling, in contrast with the effects on PDGF-mediated signaling, NT-3-stimulated Erk1/2 phosphorylation was not affected by co-exposure to TH, BMP-4, or FGF-2. Thus, the effects of these agents are pathway-specific, and the redox/Fyn/c-Cbl pathway is involved in the effects of these modulating agents.

To test the specificity of the effect of modulating agents on c-Cbl targets, rat O-2A/OPCs were exposed to TH (1:1000), BMP4 (10 ng/ml), FGF-2 (10 ng/ml) or NT-3 (10 ng/ml) for 24 hr in presence of either PDGF (10 ng/ml), HGF (10 ng/ml) or NT-3 (10 ng/ml). Cells were lysed for Western Blot using anti-PDGFRα, c-MET or TrkC antibody, respectively. The membranes were de-probed and then re-probed with anti-β-tubulin antibody. FIG. 24 shows that TH, BMP, NT-3 and FGF-2 all converge on regulation of the fyn-c-Cbl pathway, and that exposure to O-2A-OPC cells to these signaling molecules cause changes in levels of PDGFRα. TH and BMP, differentiation inducers, activated fyn and c-Cbl, leading to reductions in absolute levels of PDGFRα. Enhancers of self-renewal, FGF2 and NT-3, had opposite effects. Furthermore, there was no change in levels of the non-c-Cbl target c-Met.

TH, BMP-4, FGF-2 and NT-3 modified c-Cbl phosphorylation consistent with their functions as inducers or proliferation or differentiation. Rat O2A cells were exposed to TH (1:1000), BMP4 (10 ng/ml), FGF-2 (10 ng/ml) or NT-3 (10 ng/ml) for 3-4 hr. Cells were lysed for immunoprecipitation using anti-phospbo-tyrosine antibody, and the immunoprecipitated samples were run on a SDS-PAGE gel and blotted with anti-c-Cbl antibody. For the co-immunoprecipitation assay, anti-p-Tyr monoclonal antibody (Santa Cruz) was added into the pre-cleared cell lysates (250 μg of total protein) and the mixtures were gently rocked for 2 hr at 4° C. 30 μl of protein A/G agarose was then added into the mixture followed by rotating at 4° C. for overnight. The protein A/G agarose was then spun down and washed thoroughly three times. The precipitates were resolved on an 8% SDS-PAGE gel and subjected to Western blot analysis using an anti-Cbl antibody (BD Pharmingen). Exposure of O-2A/OPCs (induced to divide by PDGF) to TH or BMP causes increased phosphorylation of c-Cbl (See FIG. 25A), consistent with the increase in oxidative state and activation of fyn (which phosphorylates c-Cbl) caused by these agents. FGF-2 or NT-3, however, caused a reduction in c-Cbl phosphorylation (FIG. 25B). Thus, opposing signals converge on regulation of c-Cbl as a critical component of controlling cell division.

In FIG. 26, FGF-indueed increases in PDGFRα levels are shown to be independent on gene transcription. Rat O2A/OPCs grown in the presence of PDGF (10 ng/ml) were exposed to FGF-2 (10 ng/ml) for 24 hr in the presence of Ipg/ml of achromycin D. Cells were lysed for Western Blot using PDGFRα antibody. The membranes were de-probed and then re-probed with β-tubulin antibody. Cells exposed to TH showed lower levels of PDGFRα. As it had been previously reported that exposure of O-2A/OPCs to FGF-2 caused increases in transcription of the PDGFRα gene, increases in PDGFRα levels caused by PGP-2 were tested and found to be dependent on gene transcription. O-2A/OPCs co-exposed to FGF-2 and PDGF in the presence of actinomycin D, however, still show increased levels of PDGFRα, as predicted by the hypothesis that the effects seen are due to control of receptor degradation by c-Cbl activity.

### Example 4

### Protection Against the Adverse Effects of Ethanol by Inhibition of the Redox/Fyn/c-Cbl Pathway

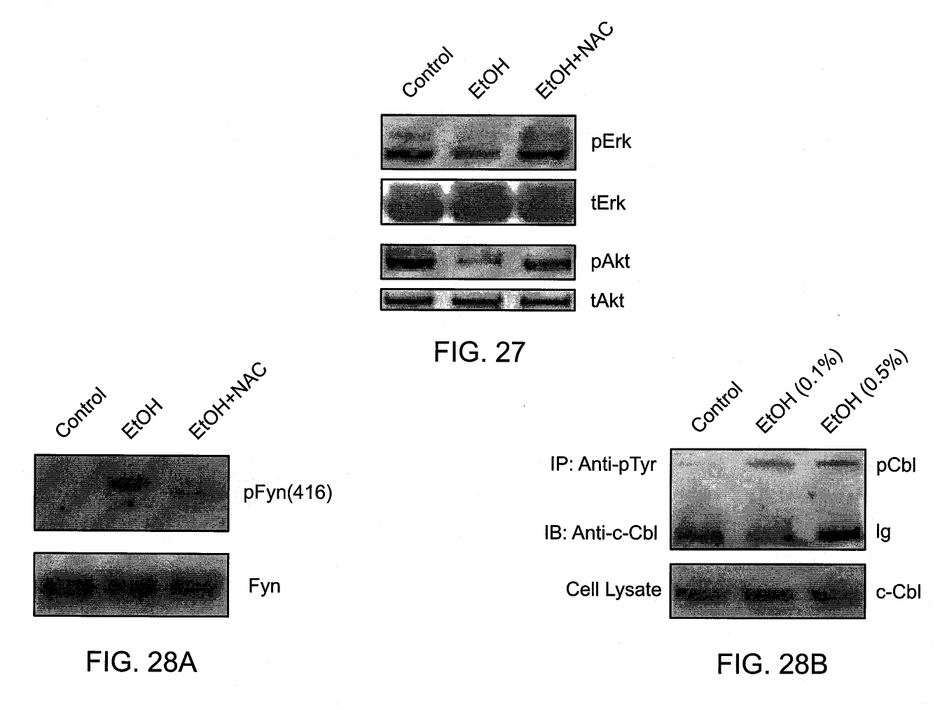

Another example of the generality of the principles of the redox//Fyn/c-Cbl pathway is shown by studies on ethanol (EtOH), showing EtOH activates the Fyn/c-Cbl pathway. As shown in FIG. 27, exposure of O-2A/OPCs to EtOH was associated with suppression of PDGF-mediated activation of Erk1/2 and Akt, and this suppression blocked by NAC. More specifically, exposure of O-2A/OPCs to 0.5% EtOH for 16 hrs was associated with a suppression of PDGF-induced phosphorylation of Erk1/2 and of Akt. Phosphorylation of both Erk1/2 and Akt were rescued when cells were exposed to 1 mM NAC at the same time as EtOH exposure. These experiments used standard conditions for such assays, in which cells were exposed to EtOH+NAC for 16 hrs, of which the last 6 hrs was in PDGF-free medium, and then treated for 30 min with 10 ng/ml PDGF. EtOH exposure (30 min) caused Fyn phosphorylation (detected with anti-pFyn(416) antibody), which is blocked by co-exposure to NAC. (FIG. 28A). Exposure to 0.1% or 0.5% EtOH caused c-Cbl phosphorylation, as detected by immunoprecipitation from lysates of treated O-2A/OPCs, followed by analysis with anti-phosphotyrosine antibody. (FIG. 28B).

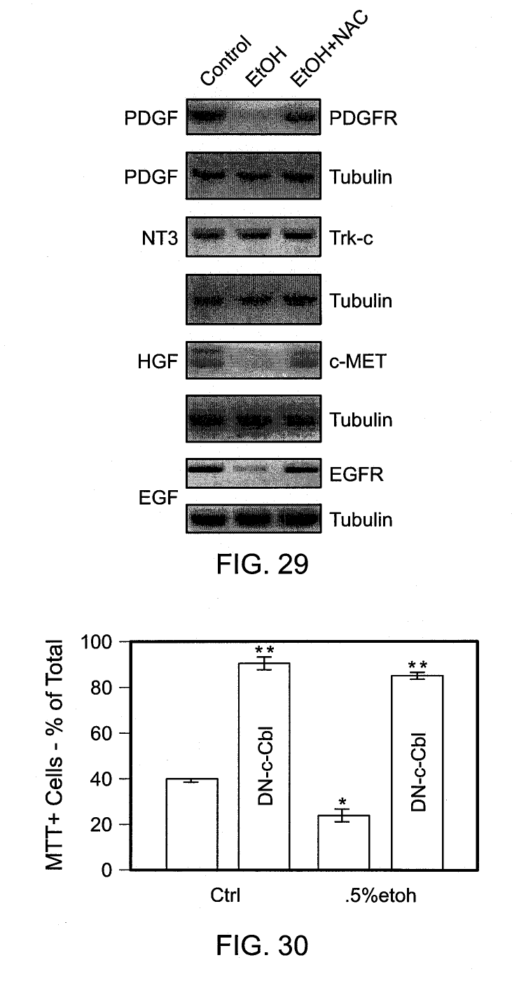

This EtOH effect was specific for c-Cbl targets. Exposure of O-2A/OPCs to EtOH resulted in reduced levels of RTKs that are c-Cbl targets, but not of TrkC. For these experiments, purified O-2A/OPCs were first grown in the presence of PDGF. They were then switched to medium containing PDGF, NT-3, HGP or EGF (ligands for PDGFRα, TrkC, c-Met or EGFR, respectively, and either grown in control medium or medium containing 0.5% EtOH for 16 hrs. In these experiments, NAC was added 1 hr before EtOH. As shown in FIG. 29, exposure to EtOH was associated with decreased levels of PDGFRα, as well as of the c-Cbl targets of c-Met and EGFR. In contrast, levels of TrkC, which is not a c-Cbl target, were unaltered by EtOH exposure. The lack of change in levels of TrkC demonstrated that it is not sufficient to simply expose cells, in the presence of EtOH, to an appropriate ligand for the RTK of interest in order to cause reductions in receptor levels. That changes in levels of RTKs that are c-Cbl targets was dependent on changes in oxidative state was indicated by the ability of NAC to antagonize the effects of EtOH and provide substantial rescue to levels of PDGFRα, c-Met and EGFR.

Expressions of dominant-negative c-Cbl blocked the EtOH-induced cell death, O-2A/OPCs were infected with retrovirus encoding DN(70Z)-c-Cbl, or empty (puromycin resistance) virus. As described above, pJEN/neo-HA-70z-c-Cbl plasmids were constructed by transferring the BamH1 digested HA-70z-c-Cbl from pjEN/neo-HA-70z-c-Cbl into the BamH1 digested pBabe(puro) vector. The plasmids were transfected into Pheonix Ampho cells by Fugene6 (Roche) transfection solution according to the manufacturer's protocol. Twenty-four hr after transfection, medium was changed into DMEM/F12(SATO-) supplemented with 10 ng/ml PDGF-AA. Virus supernatant was collected forty-eight hr post-transfection, filtered through 0.45 um filter to remove non-adherent cells and cellular debris, frozen in small aliquots on dry ice, and stored at −80° C. Twenty-four hr prior to infection with either DN-c-Cbl virus or backbone virus, O-2A cells were seeded. The following day, the culture media was aspirated and replaced with virus supernatant diluted 1:1 in the O-2A growth media. Media was then changed into O-2. A growth media after eight hr or overnight. Twenty-four hr after infection, the cells were collected by trypsinization and reseeded in the selective media (growth media+200 ng/ml puromycin). By the next day, all non-infected cells were floating and presumably dead or dying. The infected cells were allowed to proliferate for two days, and then collected and re-seeded for these experiments. (*=p<0.05; **=p<0.01 for comparison with untreated controls; there is no significant difference between DN-c-Cbl cells in control cultures or exposed to 0.5% EtOH for 72 hrs). As shown in FIG. 30, exposure of cells infected with empty virus was associated with an ˜25% decrease in the proportion of cells that were MTT+ (which, in these particular experiments, represented about 40% of total cells in control cultures). DN-c-Cbl protected against the reduction in the proportion of viable cells in EtOH-treated cultures. It is also important to note that expression of DN-c-Cbl also greatly suppressed background cell death in untreated cultures. This observation provides additional support for the general principle that activation of the Fyn/c-Cbl pathway is central to the induction of cell death.

### Example 4

### Blocking the Oxidative Effects of Amyloid Beta by Interrupting the Redox/Fyn/c-Cbl Pathway

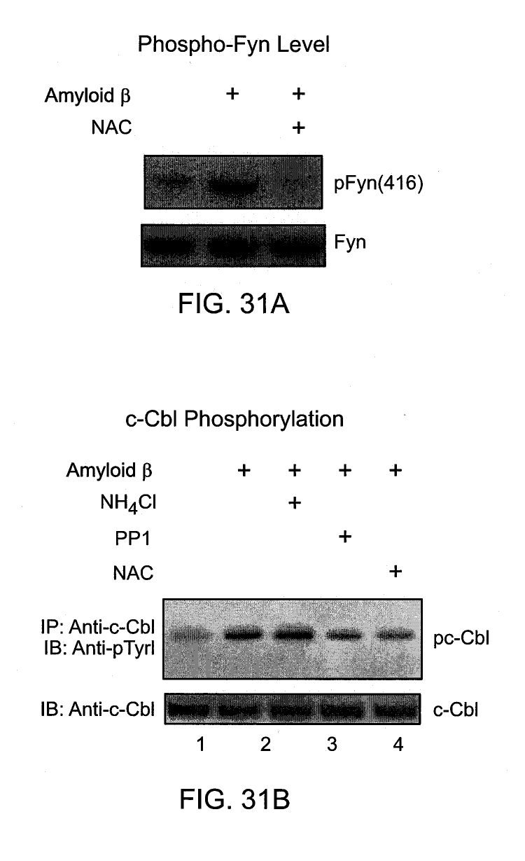

The effect of amyloid beta protein (Aβ) on the Fyn/c-Cbl pathway in O-2A/OPCs was investigated. As a threshold matter, studies were performed to establish O-2A/OPCs are responsive to Aβ exposure. O-2A/OPCs toxicity was similar to that previously reported for oligodendrocytes. Progenitor cell death was induced by concentrations of Aβ(1-42) (i.e., amino acids 1-42 of amyloid beta protein) of 20 μM, with little cell death induced over 48 hrs to 5-10 μM Aβ(1-42). See FIG. 31. Both C-Cbl and Fyn were phosphorylated by exposure of cells to Aβ. Phosphorylation was blocked by co-exposure of cells to NAC. Phosphorylation of c-Cbl was not blocked, however, by co-exposure of the cells to Nh₄Cl, which inhibits lysosomal function and would be relevant only to levels of PDGFRα and not to activation of c-Cbl.

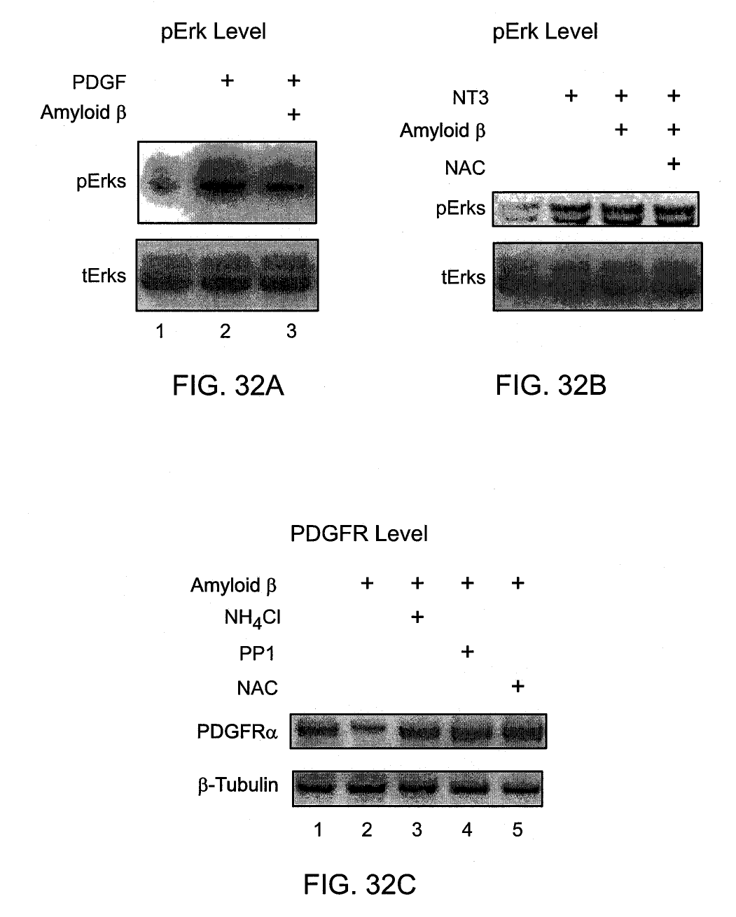

Consistent with the above Examples, exposure of O-2A/OPCs to Aβ(1-42) inhibited PDGF-mediated induction of Erk1/2 phosphorylation. See FIG. 32. This suppression is blocked by NAC and PP1, but not by NH₄Cl. This outcome is as predicted from the expected rescue of PDGFR levels by lysosomal inhibition as shown in the toxicant Examples above. In contrast with the effects of Aβ(1-42) on PDGF-mediated signaling, exposure had no effect on Erk1/2 phosphorylation induced by NT-3, which is consistent with the fact that TrkC (the receptor for NT-3) does not appear to be a c-Cbl target.

### Example 5

### In Vivo Analysis of the Effect of Chronic Oxidation on the Redox/Fyn/c-Cbl Pathway

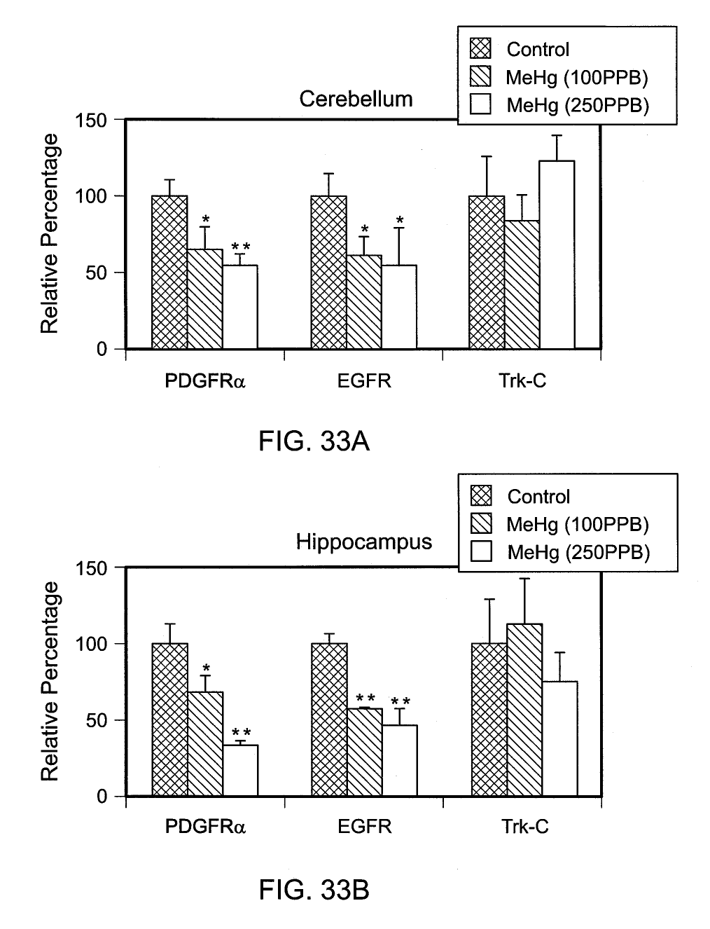

Developmental exposure of mice to MeHg at either 100 ppb or 250 ppb in the maternal drinking water was associated with clear and significant reductions in levels of PDGFRα and EGFR1 but not of TrkC (FIG. 33). Treatment of SJL mice with 100 or 250 ppb MeHg in the drinking water during gestation and suckling was associated with reductions in levels of PDGFRα and EGFR in the cerebellum, hippocampus and corpus callosum when brain tissue was sampled at 7 and 21 days after birth. In contrast, levels of the NT-3 receptor TrkC were not reduced in these animals. It was particularly striking that exposure even to 100 ppb MeHg in the drinking water was enough to have significant effects on levels of PDGFRα and EGFR. These changes, and the lack of effect of MeHg exposure on TrkC levels, was consistent with the in vitro analyses described above.

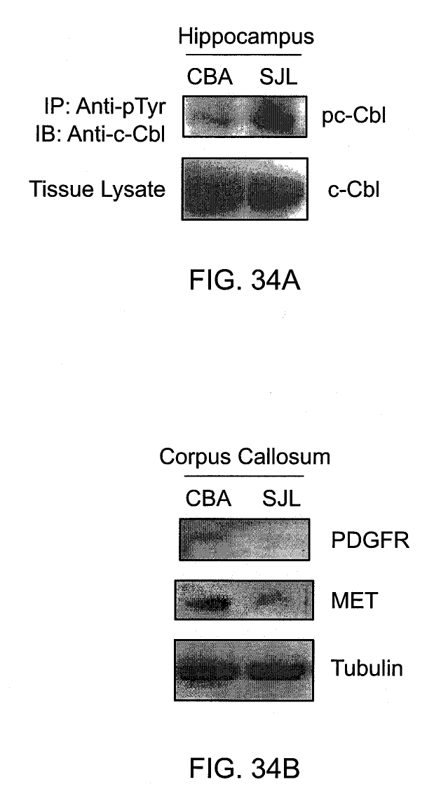

Similar results were found when strains of mice with genetically different levels of oxidation were used. More specifically, basal levels of c-Cbl activation mid of levels of c-Cbl target proteins were compared in brains of SJL mice and brains of CBA mice (which are reduced more than their SJL counterparts). Representative data from these experiments are shown in FIG. 34A, demonstrating that, in the hippocampus of SJL mice, c-Cbl is more highly phosphorylated than in the hippocampus of CBA mice. This figure also shows that in the corpus callosum of postnatal day 14 SJL mice, there are markedly reduced levels of PDGFRα and c-Met as compared with tissue from CBA mice. See FIG. 342B.

### REFERENCES

1. Stahnke T, Richter-Landsberg C (2004) Triethyltin-induced stress responses and apoptotie cell death in cultured oligodendrocytes. Glia 46; 334-344.

2. Deng W, Poretz R D (2002) Protein kinase C activation is required for the lead-induced inhibition of proliferation and differentiation of cultured oligodendroglial progenitor cells. Brain Res 929: 87-95.

3. Deng W, McKinnon R D, Poretz R D (2001) Lead exposure delays the differentiation of oligodendroglial progenitors in vitro, and at higher doses induces cell death. Toxicol Appl Pharmacol 174: 235-244.

4. Bichenkov E, Ellingson J S (2001) Ethanol exerts different effects on myelin basic protein and 2′,3′-cyclic nucleotide 3′-phosphodiesterase expression in differentiating CG-4 oligodendrocytes. Brain Res Dev Brain Res 128: 9-16.

5. Zoeller R T, Butnariu O V, Fletcher D L, Riley E P (1994) Limited postnatal ethanol exposure permanently alters the expression of mRNAS encoding myelin basic protein and myelin-associated glycoprotein in cerebellum. Alcohol Clin Exp Res 18; 909-916.

6. Harris S J, Wfice P, Bedi K S (2000) Exposure of rats to a high but not low dose of ethanol during early postnatal lite increases the rate of loss of optic nerve axons and decreases the rate of myelination. J Anat 197 (Prt 3): 477-485.

7. Özer E, Saraioglu S. Güre A (2000) Effect of prenatal ethanol exposure on neuronal migration, neurogenesis and brain myelination in the mice brain. Clin Neuropathol 19: 21-25.

8. O'Callaghan J P, Miller D B (1983) Acute postnatal exposure to triethyltin in the rat: effects on specific protein composition of subcellular fractions from developing and adult brain. J Pharmacol Exp Ther 224: 466-472.

9. Smith J Ladi E, Mayer-Pröschei M, Noble M (2000) Redox state is a central modulator of the balance between self-renewal and differentiation in a dividing glial precursor cell. Proc Natl Acad Sci USA 97: 10032-10037.

10. Noble M, Murray K, Stroobant P, Waterfield M D, Riddle P (1988) Platelet-derived growth factor promotes division and motility and inhibits premature differentiation of the oligodendrocyte/type-2 astrocyte progenitor cell. Nature 333: 560-562.

11. Richardson W D, Pringle N, Mosley M, Westermark B, Dubois-Dalcq M (1988) A role for platelet-derived growth factor in normal gliogenesis in the central nervous system. Cell 53: 309-319.

12. Calver A, Hall A, Yu W, Walsh F, Heath J, et al. (1998) Oligodendrocyte population dynamics and the role of PDGF in vivo. Neuron 20: 869-882.

13. Barres B A, Lazar M A, Raff M C (1994) A novel role for thyroid hormone, glucocorticoids and retinoic acid in timing oligodendrocyte development. Development 120: 1097-1108.

14. Ibarrola N, Mayer-Proschel M, Rodriguez-Pena A, Noble M (1996) Evidence for the existence of at least two timing mechanisms that contribute to oligodendrocyte generation in vitro. Dev Biol 180: 1-21.

15. Grinspan J B, Edell E, Carpio D F, Beesley J S, Lavy L, et al. (2000) Stage-specific effects of bone morphogenetic proteins on the oligodendrocyte lineage. J Neurobiol 43:1-17.

16. Mabie P, Mehler M, Marmur R, Papavasiliou A, Song Q, et al. (1997) Bone morphogenetic proteins induce astroglial differentiation of oligodendroglial-astroglial progenitor cells. Neurosci 17: 4112-4120.

17. Castoldi A F, Barni S, Turin I, Gandini C, Manzo L (2000) Early acute necrosis, delayed apoptosis and cytoskeletal breakdown in cultured cerebellar granule neurons exposed to methylmercury. J Neurosci Res 60: 775-787.

18. Park S T, Lim K T, Chung Y T, Kim S U (1996) Methylmercury induced neurotoxicity in cerebral neuron culture is blocked by antioxidants and NMDA receptor antagonists, Neurotoxicology 17: 37-46.

19. Aschner M, Yao C P, Allen J W, Tan K H (2000) Methylmercury alters glutamate transport in astrocytes. Neurochem Int 37: 199-206.

20. Markowski V P, Fiaugher C B, Baggs K B, Rawleigh R C, Cox C, Weiss B (1998) Prenatal and lactational exposure to methylmercury affects select parameters of mouse cerebellar development. Neurotoxicology 19: 879-892. Park S T, Lim K T, Chung Y T, Kim S U (1996) Methylmercury-induced neurotoxicity in cerebral neuron culture is blocked by antioxidants and NMDA receptor antagonists. Neurotoxicology 17: 37-46.

21. Peckham N H, Choi B H (1988) Abnormal neuronal distribution within the cerebral cortex after prenatal, methylmercury intoxication. Acta Neuropathol 76: 222-226.

22. Kakita A, Inenaga C. Sakamoto M, Takahashi H (2002) Neuronal migration disturbance and consequent cytoarchitecture in the cerebral cortex following transplacental administration of methylmercury. Acta Neuropathol (Berl) 104; 409-417.

23. Faustman E M, Ponce R A, Ou Y C, Mendoza M A, Lewandowski T, et al. (2002) Investigations of methylmercury-induced alterations in neurogenesis. Environ Health Perspect 110; 859-864.

24. Choi B H (1986) Methylmercury poisoning of the developing nervous system: I. Pattern of neuronal migration in the cerebral cortex, Neurotoxicology 7: 591-600.

25. Raff M C, Lillien L E, Richardson W D, Borne J F, Noble M D (1988) Platelet-derived growth factor from astrocytes drives the clock that times oligodendrocyte development in culture. Nature 333: 562-565.

26. Lamballe F, Klein R, Barbacid M (1991) trkC, a new member of the trk family of tyrosine protein kinases, is a receptor for neurotrophin-3. Cell 66: 967-979.

27. Miyake S, Mullane-Robinson K P, Lill N L, Douillard P, Band H (1999) Cbl-mediated negative regulation of platelet-derived growth factor receptor-dependent cell proliferation. A critical role for Cbl tyrosine kinase-binding domain, J Biol Chem 274: 16619-16628.

28. Miyake S, Lupher M L J, Drake B, Band H (1998) The tyrosine kinase regulator Cbl enhances the ubiquitination and degradation of the platelet-derived growth factor receptor alpha. Proc Natl Acad Sci U S A 95: 7927-7932.

29. Duan L, Miura Y, Dimri M, Majumder B, Dodge I L, et al. (2003) Cbl-mediated ubiquitinylation is required for lysosomal sorting of epidermal growth factor receptor but is dispensable for endocytesis. J Biol Chem 278: 28950-28960.

30. Rosenkranz S, Ikuno Y, Leong F L, Klinghoffer R A, Miyake S, et al. (2000) Sre family kinases negatively regulate platelet-derived growth factor alpha receptor-dependent signaling and disease progression. J Biol Chem 275: 9620-9627.

31. Schmidt M H, Dikic I (2005) The Cbl interactome and its functions. Nat Rev Mol Cell Biol 6: 907-919.

32. Tsygankov A Y, Mahajan S, Fineke J E, Bolen J B (1996 ) Specific association of tyrosine-phosphorylated c-Cbl with Fyn tyrosine kinase in T cells. J Biol Chem 271: 27130-27137.

33. Hunter S, Burton E A, Wu S C, Anderson S M (1999) Fyn associates with Cbl and phosphorylates tyrosine 731 in Cbl, a binding site for phosphatidylinositol 3-kinase, J Biol Chem 274: 2097-2106.

34. Feshchenko E A, Langdon W Y, Tsygankov A Y (1998) Fyn, Yes, and Syk phosphorylation, sites in c-Cbl map to the same tyrosine residues that become phosphorylated in activated T cells. J Biol Chem 273: 8223-8331.

35. Kassenbrock C K, Hunter S F, Garl P, Johnson G L, Anderson S M (2002) inhibition of Src family kinases blocks epidermal growth factor (EGF)-induced activation of Akt, phosphorylation of c-Cbl, and ubiquitination of the EGF receptor. J Biol Chem 277: 24967-24975

36. Abe J, Berk B C (1999) Fyn and JAK2 mediate ras activation by reactive oxygen species. J Biol Chem 274: 21003-21010.

37. Abe J, Okuda M, Huang Q, Yoshizumi M, Berk B C (2000) Reactive oxygen species activate p90 ribosomal S6 kinase via Fyn and Ras. J Biol Chem 275: 1739-1748.

38. Sanguinetti A R, Cao H, Corley Mastick C (2003) Fyn is required for oxidative- and hyperosmotic-stress-induced tyrosine phosphorylation of caveolin-1, Biochem J 376: 159-168.

39. Hehner S P, Breitfreutz R, Shubinsky G, Unsoeld H, Sehulze-Osthoff K, et al. (2000) Enhancement of T cell receptor signaling by a mild oxidative shift in the intracellular thiol pool. J Immunol 165: 4319-4328.

40. Osterhout D J, Wolven A, Wolf R M, Resh M D, Chao M V (1999) Morphological differentiation of oligodendrocytes requires activation of Fyn tyrosine kinase. J Cell Biol 145; 1209-1218.

41. Wolf R M, Wilkes J J, Chao M V, Resh M D (2001) Tyrosine phosphorylation of p190 RhoGAP by Fyn regulates oligodendrocyte differentiation. J Neurobiol 49: 62-78.

42. Taher T E, Tjin E P, Beuling E A, Borst J, Spaargaren M, et al. (2002) c-Cbl is involved in Met signaling in B cells and mediates hepatocyte growth factor-induced receptor ubiquitination. J Immunol 169: 3793-3780.

43. Thien C B, Langdon W Y (2005) Negative regulation of PTK signalling by Cbl proteins. Growth Factors 23; 161-167.

44. van Leeuwen J E, Paik P K, Samelson L E (1999) The oncogenic 70Z Cbl mutation blocks the phosphotyrosine binding domain-dependent negative regulation of ZAP-70 by c-Cbl in Jurkat. T cells, Mol Cell Biol 19: 6652-6664.

45. Deng W, Poretz R D (2003) Oliogodendroglia in developmental neurotoxicity. Neurotoxicol 24: 161-178.

46. McCarthy S, Somayajulu M, Sikorska M, Borowy-Borowski H, Pandey S (2004) Paraquat induces oxidative stress and neuronal death; neuroprotection by water-soluble Coenzyme Q10. Toxicol Appl Pharmacol 201: 21-31.

47. Shimizu K, Maisubara K, Ohtaki K, Shiono H (2003) Paraquat leads to dopaminergic neural vulnerability in organotypic midbrain culture. Neurosci Res 46: 523-532.

48. Chen Y W, Huang C P, Tsai K S, Yang R S, Yen C C, et al. (2006) The role of phosphoinositide 3-kinase/Akt signaling in low-dose mercury-induced mouse pancreatic {beta}-cell dysfunction in vitro and in vivo. Diabetes 55: 1614-16124.

49. Ballatori N, Lieberman M W, Wang W (1998) N-acetylcysteine as an antidote in methylmercury poisoning. Environ Health Perspect 106: 267-271.

50. Shanker G, Syversen T, Aschner M (2005) Modulatory effect of glutathione status and antioxidants on methylmercury-induced free radical formation in primary cultures of cerebral astrocytes. Brain Res Mol Brain Res 137: 11-22.

51. Nehru B, Kanwar S S (2004) N-acetylcysteine exposure on lead-induced lipid, peroxidative damage and oxidative defense system in brain regions of rats. Biol Trace Elem Res 101:257-264.

52. Neal R, Copper K, Gurer H, Ercal N (1998) Effects of N-acetyl cysteine and 2,3-dimercaptosuccinic acid on lead induced oxidative stress in rat lenses. Toxicology 130:167-174.

53. Yeh S T, Guo H R, Su Y S, Lin H J, Hou C C, et al. (2006) Protective effects of N-acetylcysteine treatment post acute paraquat intoxication in rats and in human lung epithelial cells. Toxicology 223: 181-190.

54. Satoh E, Okada M, Takadera T, Ohyashiki T (2005) Glutathione depletion promotes aluminum-mediated cell death of PC12 cells. Biol Pharm Bull 28: 941-946.

55. Tamiori S K, Singh S, Prasad S, Khandekar K, Dwivedi V K, et al. (2003) Reversal of cadmium induced oxidative stress by chelating agent, antioxidant or their combination in rat Toxicol Lett 145: 211-217.

56. Flora S J (1999) Arsenic-induced oxidative stress and its reversibility following combined administration of N-acetylcysteine and meso 2,3-dimercaptosuccinic acid in rats. Clin Exp Pharmacol Physiol 26: 865-869.

57. Zaragoza A, Diez-Fernandez C, Alvarez A M, Andres D, Cascales M (2001) Mitochondrial involvement in cocaine-treated rat hepatocytes: effect of N-acetylcysteine and deferoxamine. Br J Pharmacol 132:1063-1070.

58. Roberts J, Nagasawa H, Zera R, Fricke R, Goon D (1987) Prodrugs of L-cysteine as protective agents against acetaminophen-inducedhepatotoxicity. 2-(Polyhydroxyalkyl)- and and 2-(polyacetoxyalkyl)thiazolidine-4(R)-carboxylic acids, J Med Chem 30: 1891-1896.

59. Yan. H, Rivkees S A (2002) Hepatocyte growth factor stimulates the proliferation and migration of oligodendrocyte progenitor cells, J Neurosci Res 69: 597-606.

60. Knapp P E, Adams M B (2004) Epidermal growth factor promotes oligodendrocyte process formation and regrowth after injury. Exp Cell Res 296; 135-144.

61. Levkowitz G, Klapper L N, Tzahar E, Freywald A, Sela M, et al. (1996) Coupling of the c-Cbl protooncogene product to ErbB-1/EGF-receptor but not to other ErbB proteins. Oncogene 12:1117-1125.

135. Rubin C, Gur G, Yarden Y (2005) Negative regulation of receptor tyrosine kinases: Unexpected links to c-Cbl and receptor ubiquitylation. Cell Res 15: 66-71.

62. de Melker A A, van der Horst G, Borst J (2004) c-Cbl directs EGF receptors into an endocytie pathway that involves the ubiquitin-interacting motif of Eps15. J Cell Sci 117: 5001-5012.

63. Ravid T, Heidinger J M, Gee P, Khan E M, Goldkorn T (2004) c-Cbl-mediated ubiquitinylation is required for epidermal growth factor receptor exit from the early endosomes. J Biol Chem 279: 37153-37162.

64. Garcia-Guzman M, Larsen E, Vuori K (2000) The proto-oneogene c-Cbl is a positive regulator of Met-induced MAP kinase activation: a role for the adaptor protein Crk. J Immunol 19: 4058-4065.

65. Tiffany-Castiglioni E (1993) Cell culture models for lead toxicity in neuronal and glial cells. Neurotoxicol 14; 513-536.

66. Krigman M R. Druse M J, Traylor T D, Wilson M H, Newell L R, et al. (1974) Lead encephalopathy in the developing rat: Effect on myeiination. J Neuropathol Exp Neurol 33: 58-73.

67. Dabrowska-Bouta B, Sulkowski G, Bartosz G, Walski M, Rafalowska U (1999) Chronic lead intoxication affects the myelin membrane status in the central nervous system of adult rats. J Mol Neurosci 13: 127-139.

68. Deng W, Poretz R D (2001) Chronic dietary lead exposure affects galactolipid metabolic enzymes in the developing rat brain. Toxicol Appl Pharmacol 172: 98-107.

69. Weiss B, Stern S, Cox C, Balys M (2005) Perinatal and lifetime exposure to methylmercury in the mouse: Behavioral effects. Neurotoxicology 26: 675-690.

70. Stem S, Cox C, Cernichiari E, Balys M, Weiss B (2001) Perinatal and lifetime exposure to methylmercury in the mouse: Blood and brain concentrations of mercury to 26 months of age. Neurotoxicology 22: 467-477.

71. Gouiet S, Dore F Y, Mirault M E (2003) Neurobehavioral changes in mice chronically exposed to methylmercury during fetal and early post-natal development. Neurotoxicol Teratol 25: 335-347.

72. Sakamoto M, Kakita A, de Oliveira R B, Pan H S, Takahashi H (2004) Dose-dependent effects of methylmercury administered during neonatal brain spurt in rats. Dev Brain Res 152: 171-176.

73. Barone S Jr, Haykal-Coates N, Parran D K, Tilson H A (1998) Gestational exposure to methylmercury alters the developmental pattern of trk-like immtmoreactivity in the rat brain and results in cortical dysraorphology. Brain Res Dev Brain Res 109: 13-31.

74. Dietrich J, Han R, Yang Y, Mayer-Prosehel M, Noble M (2006) CNS progenitor cells and oligodendrocytes are targets of chemotherapeutic agents in vitro and in vivo. J Biol 5; 22 (in press).

75. Liang X, Draghi N A, Resh M D (2004) Signaling from integrins to Fyn to Rho Family GTPases regulates morphologic differentiation of oligodendrocytes. J Neurosci 24: 7140-7149.

76. Tsatmali M, Walcott E C, Crossin K L (2005) Newborn neurons acquire high levels of reactive oxygen species and increased mitochondrial proteins upon differentiation from progenitors. Brain Res 1040: 137-150.

77. Goldsmit Y. Erlich S, Pinkas-Kramarski R (2001) Neuregulin induces sustained reactive oxygen species generation to mediate neuronal differentiation. Cell Mol Neurobiol 211: 753-769.

78. Puceat M (2005) Role of Rac-GTPase and reactive oxygen species in cardiac differentiation of stem cells. Antioxid Redox Signal 7: 1435-1439.

79. Yakoviev A Y, Boucher K, Mayer-Proschel M, Noble M (1998) Quantitative insight into proliferation and differentiation of O-2A progenitor cells in vitro: The clock model revisited. Proc Natl Acad Sci U S A 95: 14164-14167.

80. Hyrien O, Mayer-Proschel M, Noble M, Yakoviev A (2005) Estimating the life-span of oligodendrocytes from clonal data on their development in cell culture. Math Biosci 193: 255-274.

81. Hyrien O, Mayer-Proschel M, Noble M, Yakoviev A (2005) A stochastic model to analyze clonal data on multi-type cell populations. Biometrics 61: 199-207.

82. Tamm C, Duckworth J, Hermanson O, Ceccatelli S (2006) High susceptibility of neural stem cells to methylmercury toxicity: Effects on cell survival and neuronal differentiation. J Neurochem 97: 69-78.

83. Fruttiger M, Karlsson L, Hall A, Abramsson A, Calver A, et al. (1999) Defective oligodendrocyte development and severe hypomyelination in PDGF-A knockout mice. Development 126; 457-467.

84. Hoch R; Soriano P (2003) Roles of PDGF in animal development. Development 130; 4769-4784.

85. Betsholtz C (2004) Insight into the physiological functions of PDGF through genetic studies in mice. Cytokine Growth. Factor Rev 15: 215-228.

86. Wong R W C. Guillaud L (2004) The role of epidermal growth factor and its receptors in mammalian CNS, Cytokine Growth Factor Rev 15: 147-156.

87. Xian C J, Zhou X F (2004) EGF family of growth factors: Essential roles and functional redundancy in the nerve system. Front Biosci 9: 85-92.

88. Holbro T, Hynes N E (2004) ErbB receptors: Directing key signaling networks throughout life. Annu Rev Pharmacol Toxicol 44: 195-217.

89. Gutierrez H, Dolcet C, Tolcos M, Davies A (2004) HGF regulates the development of cortical pyramidal dendrites. Development 131: 3717-3726.

90. Birchmeier C, Gherardi E (1998) Developmental role of HGF/SF and its receptor, the c-Met tyrosine kinase. Trends Cell Biol 8: 404-410.

91. Morita A, Yamashiia N, Sasaki Y, Uchida Y, Nakajima O, et al. (2006) Regulation of dendritic branching and spine maturation by semaphorin3A-Fyn signaling. J Neurosci 26: 2971-2980.

92. He J, Nixon K, Shetry A K, Crews F T (2005) Chronic alcohol exposure reduces hippocampal neurogenesis and dendritic growth of newborn, neurons. Eur J Neurosci 21; 2711-2720.

93. Newey S E, Velamoor V, Govek E -E, Van Aeist L (2005) Rho GTPases, dendritic structure, and mental retardation. J Neurobiol 64; 58-74.

94. Power J, Mayer-Proschel M, Smith J, Noble M (2002) Oligodendrocyte precursor cells from different brain regions express divergent properties consistent with the differing time courses of myelination in these regions. Dev Biol 245: 362-375.

95. Sakamoto M, Kakita A, Wakabayashi K, Takahashi H, Nakano A, et al. (2002) Evaluation of changes in methylmercury accumulation in the developing rat brain and its effects: A study with consecutive and moderate dose exposure throughout gestation and lactation periods. Brain Res 949: 51-59.

What is claimed is:

1. A method of reducing oxidation in a cell comprising
(a) providing the cell, wherein the cell has an activated redox/fyn/c-Cbl pathway or is at risk for activation of the redox/fyn/c-Cbl pathway:
(b) contacting the cell with an agent that selectively interrupts the redox/fyn/c-Cbl pathway, wherein interruption of the redox/fyn/c-Cbl pathway reduces oxidation as compared to oxidation in the absence of the agent.

2. The method of claim 1, wherein the agent that selectively interrupts the redox/Fyn/c-Cbl pathway reduces activation of Fyn kinase, interaction of Fyn kinase and c-Cbl ubiquitin ligase, c-Cbl ubiquitin ligase activation, or reduces c-Cbl ubiquitin ligase interaction with a target, as compared to activation of Fyn kinase, interaction of Fyn kinase and c-Cbl ubiquitin ligase, c-Cbl ubiquitin ligase activation, or interaction of c-Cbl ubiquitin ligase interaction in a control.

3. The method of claim 1, wherein the activation of the redox/fyn/c-Cbl pathway is caused by a chemotherapeutic agent.

4. The method of claim 1, wherein the activation of the redox/fyn/c-Cbl pathway is caused by hyperglycemia.

5. The method of claim 1, wherein the activation of the redox/fyn/c-Cbl pathway is caused by a toxicant.

6. The method of claim 1, wherein the activation of the redox/fyn/c-Cbl pathway is caused by ethanol.

7. The method of claim 1, wherein the activation of the redox/fyn/c-Cbl pathway is caused by amyloid β.

8. The method of claim 1, wherein the cell is a precursor cell.

9. The method of claim 8, wherein the precursor cell is a neural progenitor cell.

10. The method of claim 9, wherein the neural progenitor cell is an O-2A cell.

11. The method of claim 8, wherein the precursor cell is a pancreatic islet cell progenitor.

12. The method of claim 1, wherein the cell is an insulin-producing cell.

13. The method of claim 1, wherein the cell is a neuron.

14. The method of claim 1, wherein the contacting step is in vivo.

15. The method of claim 1, wherein the contacting step is in vitro.

16. The method of claim 1, wherein the agent is a c-Cbl inhibitor.

17. The method of claim 16, wherein the agent is an siRNA.

18. The method of claim 1, wherein the agent is selected from the group consisting of an anti-oxidant, an agent that increases glutathione levels, a cysteine pro-drug and an Fyn inhibitor.

19. A method of screening for a pro-oxidation toxicant comprising
(a) contacting a precursor cell with an agent to be tested for pro-oxidative toxic effects;
(b) detecting activation of a redox/fyn/c-Cbl pathway, activation of the redox/fyn/c-Cbl pathway indicating the agent has pro-oxidation toxic effects.

20. The method of claim 19, wherein detection of activation of the redox/lyn/c-Cbl pathway comprises detection of Fyn kinase activation, of c-Cbl ubiquitin ligase activation, or of the level of one or more c-Cbl targets.

21. The method of claim 19, wherein detection of the activation of the redox/fyn/c-Cbl pathway comprises detecting downregulation of one or more selective targets of c-Cbl ubiquitin ligase.

22. The method of claim 21, wherein the selective target of c-Cbl ubiquitin ligase is a receptor tyrosine kinase.

23. The method of claim 22, wherein, the receptor tyrosine kinase is selected from the group consisting of PDGFRα EGFR, and c-Met.

24. The method, of claim 19, wherein detection of the activation of the redox/fyn/c-Cbl pathway further comprises detecting the absence of downregulation of a non-c-Cbl ubiquitin ligase target.

25. The method of claim 24, wherein the a non-c-Cbl ubiquitin ligase target is TrkC.

26. The method of claim 19, wherein the precursor cell is a neural progenitor cell.

27. The method of claim 26, wherein the neural progenitor cell is an O-2A cell.

28. A method of determining a concentration of a toxicant that has pro-oxidative effects.
(a) contacting one or more precursor cells with one or more concentrations of the toxicant to be tested;
(b) detecting the level of activation of a redox/fyn/c-Cbl pathway at each concentration,
activation above control levels indicating the concentration has pro-oxidative effects.

29. A method of promoting proliferation of a precursor cell comprising contacting the O-2A cell with an. agent that selectively interrupts the redox/fyn/c-Cbl pathway.

30. A method of promoting differentiation of a progenitor cell or its progeny comprising contacting the O-2A cell with an agent that selectively activates the redox/fyn/c-Cbl pathway.

31. A method of treating a subject with cancer comprising the steps of
(a) administering to the subject a chemotherapeutic agent
(b) administering to the subject an agent that selectively interrupts the redox/fyn/c-Cbl pathway.

32. A method of detecting an oxidized state in a cell comprising
(a) detecting the level of one or more c-Cbl targets and
(b) detecting the level of one or more non-Cbl targets,
a reduced level of one or c-Cbl targets, as compared to a control cell, in the absence of a reduced level of the non-Cbl target indicating the oxidized state in the cell.

33. A method of testing oxidation levels in a subject comprising
(a) detecting the level of one or more c-Cbl targets in a biological sample from the subject and
(b) detecting the level of one or more non-Cbl targets in the biological sample,
a reduced level of one or c-Cbl targets, as compared to control level in the absence of a reduced level of the non-Cbl target indicating an oxidized state in the subject.

34. The method of claim 33, wherein the subject is selected as being at risk for a highly oxidized state.

35. The method of claim 34, wherein the subject is at risk for autism.

36. The method of claim 34, wherein the subject is at risk for Alzheimer's disease.

37. The method of claim 34 wherein the subject is at risk for exposure to environmental toxicants.

38. The method of claim 33, further comprising selecting a treatment for the subject based on the subject's oxidation level.
